(19) United States
(12) Patent Application Publication
(10) Pub. No.: US 2023/0042198 A1
(43) Pub. Date: Feb. 9, 2023

(54) Methods and Compositions for Modulationg Heterochromatin Dysfunction, Genomic Instability, and Associate Conditions

(71) Applicant: La Jolla Institute for Immunology, La Jolla, CA (US)

(72) Inventors: Anjana Rao, La Jolla, CA (US); Isaac F. Lopez-Moyado, La Jolla, CA (US)

(21) Appl. No.: 17/779,392

(22) PCT Filed: Nov. 25, 2020
(86) PCT No.: PCT/US2020/062243

§ 371 Date: May 24, 2022

Related U.S. Application Data
(60) Provisional application No. 62/939,794, filed on Nov. 25, 2019.

(51) Int. Cl. A61K 38/44; C07K 16/40; A61K 38/45; A61K 38/17; C12N 15/113

(52) U.S. Cl. CPC A61K 38/44; C07K 16/40; A61K 38/45; A61K 38/1709; C12N 15/1137; C12N 2310/14

(57) ABSTRACT

The present invention includes a method of increasing, stimulating, inducing, promoting, enhancing or maintaining the genomic stability of a cell of a subject, the method comprising decreasing, reducing, inhibiting, suppressing, limiting or controlling loss of methylation of heterochromatin in the cell and/or modulating heterochromatin dysfunction in a cell of a subject, the method comprising activating, eliciting, stimulate ng, inducing, promoting, increasing or enhancing expression or activity in the cell of one or more DNA methyltransferase (DNMT).

### CROSS-REFERENCE TO RELATED APPLICATIONS

This application claims priority to U.S. Provisional Application Ser. No. 62/939,794, filed Nov. 25, 2019, the entire contents of which are incorporated herein by reference.

### STATEMENT OF FEDERALLY FUNDED RESEARCH

This invention was made with government support under contract/grant number R35 CA210043 awarded by NIH. The Government has certain rights in the invention.

### TECHNICAL FIELD OF THE INVENTION

The invention relates to the field of treatment, prevention, and/or reduction of heterochromatin dysfunction, genomic instability, and associated conditions and/or diseases, including cancer, age-associated genome dysfunctions, immune disorders, or autoimmune response, disorder or diseases, by increasing or enhancing expression or activity in a subject cell of one or more DNA methyltransferase (DNMT), specifically at the heterochromatin of the cell.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2020, is named ______.txt and is, ______ bytes in size.

### BACKGROUND OF THE INVENTION

Without limiting the scope of the invention, its background is described in connection with cancer. Cancer genomes are characterized by focal increases in DNA methylation, co-occurring with widespread hypomethylation. Herein it is shown that TET loss-of-function results in a similar genomic footprint. Both 5hmC in wildtype genomes, and DNA hypermethylation in TET-deficient genomes, are largely confined to the active euchromatic compartment, consistent with the known functions of TET proteins in DNA demethylation and the known distribution of 5hmC at transcribed genes and active enhancers. In contrast, an unexpected DNA hypomethylation noted in multiple TET-deficient genomes is primarily observed in the heterochromatin compartment. In a mouse model of T cell lymphoma driven by TET deficiency (Tet2/3 DKO T cells), genomic analysis of malignant T cells revealed DNA hypomethylation in the heterochromatic genomic compartment, as well as reactivation of repeat elements and enrichment for single nucleotide alterations, primarily in heterochromatic regions of the genome.

Moreover, hematopoietic stem/precursor cells (HSPC) doubly deficient for Tet2 and Dnmt3a displayed greater losses of DNA methylation than HSPC singly deficient for Tet2 or Dnmt3a alone, explaining the unexpected synergy between DNMT3A and TET2 mutations in myeloid and lymphoid malignancies. Tet1-deficient cells showed decreased localization of Dnmt3a in the heterochromatin compartment compared to WT cells, pointing to a functional interaction between TET and DNMT proteins and providing an explanation for the hypomethylation observed in TET-deficient genomes. These data provide that TET loss-of-function may at least partially underlie the characteristic pattern of global hypomethylation coupled to regional hypermethylation observed in diverse cancer genomes and highlight the contribution of heterochromatin hypomethylation to oncogenesis.

### SUMMARY OF THE INVENTION

In one embodiment, the present invention includes a method of increasing, stimulating, inducing, promoting, enhancing or maintaining the genomic stability of a cell of a subject, the method comprising decreasing, reducing, inhibiting, suppressing, limiting or controlling loss of methylation of heterochromatin in the cell.

In another embodiment, the present invention includes a method of modulating heterochromatin dysfunction in a cell of a subject, the method comprising activating, eliciting, stimulating, inducing, promoting, increasing or enhancing expression or activity in the cell of one or more DNA methyltransferase (DNMT) or one or more TET methyl-cytosine dioxygenases (TET) proteins, or both. In one aspect, the method further comprises increasing the activity of, or overexpressing: one or more DNMTs, USP7, one or more TET methyl-cytosine dioxygenases (TET) proteins, increased expression of one or more DMNTs, USP7, or TETs by CRISPRa, Vitamin C, expression of one or more heterochromatin-targeted DNMTs, or expression of one or more heterochromatin-targeted. In another aspect, the method comprises activating, eliciting, stimulating, inducing, promoting, increasing or enhancing expression or activity of: one or more DNA methyltransferase (DNMT) or one or more TET methyl-cytosine dioxygenases (TET) proteins, in the cell by administering to the subject an effective amount of an agent that increases the expression or activity of the one or more DNMTs or TETs. In another aspect, the method comprises restoring methylation, reducing defective chromosome segregation, reducing undesired cell proliferation, differentiation, or migration, or reducing heterochromatin aberrations, centromere aberrations, telomere aberrations, R-loops, G-quadruplexes, DNA damage, aneuploidies or cell defects or undesired cell proliferation, differentiation, or migration. In another aspect, the method comprises decreasing, reducing, inhibiting, suppressing, limiting or controlling a heterochromatin dysfunction or genomic instability. In another aspect, the method comprises decreasing, reducing, inhibiting, suppressing, limiting or controlling an adverse symptom of the heterochromatin dysfunction or genomic instability in the subject. In another aspect, the adverse symptom of the heterochromatin dysfunction or genomic instability in the subject heterochromatin aberrations, centromere aberrations, telomere aberrations, R-loops, G-quadruplexes, DNA damage, aneuploidies or cell defects or undesired cell proliferation, differentiation, or migration. In another aspect, the cell is at least one of: a cancer cell, a cell with one or more unstable chromosomes, an aged cell, or a senescent cell. In another aspect, the method comprises decreasing, reducing, inhibiting, suppressing, limiting or controlling an adverse symptom of a neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy. In another aspect, the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy. In another aspect, the heterochromatin dysfunction or genomic instability results in an undesirable or aberrant age-associated genome dysfunction, immune disorder or autoimmune response, disorder or disease. In another aspect, the method comprises decreasing, reducing, inhibiting, suppressing, limiting or controlling an adverse symptom of the undesirable or aberrant age-associated genome dysfunction, immune disorder, or autoimmune response, disorder or disease in the subject. In another aspect, the adverse symptom is chronic or acute. In another aspect, the undesirable or aberrant age-associated genome dysfunction, immune disorder, or autoimmune response, disorder or disease, or symptom thereof, comprises hearing loss, presbycusis, increased cerumen production, loss of visual acuity, visual impairment, loss of vestibular function, sarcopenia, chronic inflammation, declining hormone levels, impaired muscle mitochondrial function, impaired muscle stem cell function, muscle weakness, immunosenescence, decrease in urologic function, cardiovascular disease, chronic ischemic heart disease, congestive heart failure, arrhythmia, atherosclerosis, peripheral vascular disease, hypertension, rheumatoid arthritis, juvenile rheumatoid arthritis, osteoarthritis, osteoporosis, short-term memory loss, dementia, Alzheimer's disease, progerias, Hutchinson-Gilford progeria syndrome (HGPS), Werner syndrome (WS), Cockayne syndrome (CS), Bloom syndrome (BS), ataxia-telangiectasia (A-T), xeroderma pigmentosum (XP), Rothmund-Thomson syndrome (RTS), centromere instability, telomere instability, facial anomalies syndrome (ICF), myelodysplasia syndrome (MDS), chronic lymphocytic leukemia (CLL), and acute myeloid leukemia (AML), psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome. In another aspect, the one or more DNMT is selected from DNMT1, DNMT3a, or DNMT3b, or an ortholog, homologue, variant, fragment, subsequence or derivative thereof. In another aspect, the agent is selected from the group of: an agent that promotes the activity of the one or more DNMTs or TETs at the heterochromatin in the cell; an agent that transports the one or more DNMT or TETs to the heterochromatin in the cell; an agent that increases the binding of the one or more DNMT or TETs to the heterochromatin in the cell; an agent that activates the expression of the one or more DNMT or TETs by the cell; or an agent comprising or consisting of DNMT1, DNMT3a, or DNMT3b, or an ortholog, homologue, variant, fragment, subsequence or derivative thereof; or an agent comprising or consisting of TET1, TET2, or TET3, or an ortholog, homologue, variant, fragment, subsequence or derivative thereof. In another aspect, the agent is a small molecule, a ligand, an antibody, antibody fragment or mimetic, a protein, a fusion protein, a peptide, a nucleotide or a small interfering RNA. In another aspect, the agent is an antibody that binds to DNMT or a DNMT ligand, an agent that activates a DNMT gene, or a prodrug or solvate thereof. In another aspect, the agent modulates the one or more DNMT by promoting the trafficking of the one or more DNMTs or one or more TETs to the heterochromatin of the cell. In another aspect, the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof. In another aspect, the agent comprises heterochromatin protein 1 beta (hp1b), or an ortholog, homologue, variant, fragment, subsequence or derivative thereof. In another aspect, the agent is administered prior to, contemporaneous with, or after diagnosis or treatment of the neoplasia, neoplastic disorder, tumor, cancer or malignancy; metastasis of a neoplasia, tumor, cancer or malignancy to other sites; formation or establishment of a metastatic neoplasia, neoplastic disorder, tumor, cancer or malignancy to other sites distal from a primary neoplasia, neoplastic disorder, tumor, cancer or malignancy; or undesirable or aberrant age-associated genome dysfunction, immune disorder, or autoimmune response, disorder or disease.

In another embodiment, the present invention includes a method of treating, preventing, reducing, suppressing, alleviating, or ameliorating an age-associated genome dysfunction in a subject in need thereof, the method comprising administering to the subject an agent that increases the expression of or activity of one or more DNMTs or TET proteins, or both, in the subject. In another aspect, the one or more DNMT is selected from DNMT1, DNMT3a, or DNMT3b, or an ortholog, homologue, variant, fragment, subsequence or derivative thereof. In another aspect, the agent increases the activity of the one or more DNMTs, USP7, or TET proteins by promoting the activity of the one or more DNMTs, USP7, or TETs at a heterochromatin in the subject. In another aspect, the agent is a DNMT or TET agonist. In another aspect, the agent increases the activity of the one or more DNMT or TETs by promoting the trafficking of the one or more DNMT or TETs to the heterochromatin of the subject. In another aspect, the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof. In another aspect, the agent is a small molecule, a ligand, an antibody, antibody fragment or mimetic, a protein, a fusion protein, a peptide, a nucleotide or a small interfering RNA. In another aspect, the agent is an antibody that binds to DNMT or a DNMT ligand, a DNMT gene activating agent, or a prodrug or solvate thereof. In another aspect, the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof. In another aspect, the agent comprises heterochromatin protein 1 beta (hp1b), or an ortholog, homologue, variant, fragment, subsequence or derivative thereof. In another aspect, the agent is administered prior to, contemporaneous with, or after diagnosis or treatment of the undesirable or aberrant age-associated genome dysfunction.

In another embodiment, the present invention includes a method of treating, preventing, reducing, suppressing, alleviating, or ameliorating an immune disorder, or autoimmune response, disorder or disease in a subject in need thereof, the method comprising administering to the subject an agent that increases the expression of or activity of one or more DNA methyltransferases (DNMTs) proteins, an agent that increases the expression of or activity of one or more TET methyl-cytosine dioxygenases (TET) proteins, or both. In one aspect, the one or more DNMT is selected from DNMT1, DNMT3a, or DNMT3b, or an ortholog, homologue, variant, fragment, subsequence or derivative thereof; or wherein the one or more TET is selected from TET1, TET2, or TET3, or an ortholog, homologue, variant, fragment, subsequence or derivative thereof. In another aspect, the agent increases the activity of the one or more DNMTs or one or more TETs by promoting the activity of the one or more DNMT at heterochromatin in the subject. In another aspect, the agent is a DNMT or TET agonist. In another aspect, the agent increases the activity of the one or more DNMTs or TETs by promoting the trafficking of the one or more DNMTs or TETs to the heterochromatin of the subject. In another aspect, the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof. In another aspect, the agent is a small molecule, a ligand, an antibody, antibody fragment or mimetic, a protein, a fusion protein, a peptide, a nucleotide or a small interfering RNA. In another aspect, the agent is an antibody that binds to DNMT or a DNMT ligand, a DNMT gene activating agent, or a prodrug or solvate thereof. In another aspect, the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof. In another aspect, the agent comprises heterochromatin protein 1 beta (hp1b), or an ortholog, homologue, variant, fragment, subsequence or derivative thereof. In another aspect, the agent is administered prior to, contemporaneous with, or after diagnosis or treatment of the immune disorder, or autoimmune response, disorder or disease.

In another embodiment, the present invention includes a method of treating cancer in a subject in need thereof, the method comprising administering to the subject an agent that increases the expression of or activates one or more DNMT in the subject.

In another embodiment, the present invention includes a method of treating cancer in a subject in need thereof, the method comprising administering to the subject an agent that increases or promotes the activity one or more DNMT by promoting the trafficking of the one or more one or more DNA methyltransferase (DNMT) or one or more TET methyl-cytosine dioxygenases (TET) proteins, or both, to the heterochromatin in the cancer of the subject. In one aspect, the one or more DNMTs comprises or consists of DNMT1, DNMT3a, or DNMT3b, or an ortholog, homologue, variant, fragment, subsequence or derivative thereof; or one or more TETs comprises or consists of TET1, TET2, or TET3, or an ortholog, homologue, variant, fragment, subsequence or derivative thereof. In another aspect, the agent increases the activity of the one or more DNMT at the heterochromatin in the subject. In another aspect, the agent is an DNMT or TET agonist. In another aspect, the agent is selected from the group of: an agent that promotes the activity of the one or more DNMT at the heterochromatin in the subject; an agent that transports the one or more DNMT to the heterochromatin in the subject; an agent that increases the binding of the one or more DNMT to the heterochromatin in the subject; an agent that activates the expression of the one or more DNMT in the subject; or an agent comprising or consisting of DNMT1, DNMT3a, or DNMT3b, or an ortholog, homologue, variant, fragment, subsequence or derivative thereof; or an agent comprising or consisting of TET1, TET2, or TET3, or an ortholog, homologue, variant, fragment, subsequence or derivative thereof. In another aspect, the agent is a small molecule, a ligand, an antibody, antibody fragment or mimetic, a protein, a fusion protein, a peptide, a nucleotide or a small interfering RNA. In another aspect, the agent is an antibody that binds to DNMT or a DNMT ligand, a DNMT gene activating agent, or a prodrug or solvate thereof. In another aspect, the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof. In another aspect, the agent comprises heterochromatin protein 1 beta (hp1b), or an ortholog, homologue, variant, fragment, subsequence or derivative thereof. In another aspect, the agent is administered prior to, contemporaneous with, or after treatment or diagnosis of the cancer. In another aspect, the administration is local or systemic. In another aspect, the administration comprises intravenous administration. In another aspect, the subject is a mammal. In another aspect, the subject is a human patient. In another aspect, the DNMT expression or activity, the TET expression or activity, or both, is prophylactically activated, elicited, stimulated, induced, promoted, increased or enhanced to increase, stimulate, induce, promote, enhance or maintain the genomic stability of the cell of the subject, wherein the cell is at least one of: a cancer cell, a cell with one or more unstable chromosomes, an aged cell, or a senescent cell.

In another embodiment, the present invention includes a kit comprising an agent that modulates the activity of one or more DNMTs, one or more TETs, or both and instructions for use. In one aspect, the one or more DNMT is selected from DNMT1, DNMT3a, or DNMT3b, or an ortholog, homologue, variant, fragment, subsequence or derivative thereof; and the one or more TETs comprising or consisting of TET1, TET2, or TET3, or an ortholog, homologue, variant, fragment, subsequence or derivative thereof, or combinations thereof. In another aspect, the agent increases the activity of the one or more DNMTs or one or more TETs, or both, by promoting the trafficking of the one or more DNMTs, TETs, or both, to heterochromatin. In another aspect, the agent is an DNMT or TET agonist. In another aspect, the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof. In another aspect, the agent is a small molecule, a ligand, an antibody, antibody fragment or mimetic, a protein, a fusion protein, a peptide, a nucleotide or a small interfering RNA. In another aspect, the agent is an antibody that binds to DNMT or a DNMT ligand, a DNMT gene activating agent, or a prodrug or solvate thereof. In another aspect, the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof. In another aspect, the agent comprises heterochromatin protein 1 beta (hp1b), or an ortholog, homologue, variant, fragment, subsequence or derivative thereof. In another aspect, the agent is administered prior to, contemporaneous with, or after diagnosis or treatment.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show widespread DNA hypomethylation in TET-deficient mouse ESC. (FIG. 1A) Density distribution (left panels) and cumulative distribution (middle panels) of average DNA methylation values within 1 kb and 10 kb windows across the genome in wild type (WT), Dnmt TKO, and Tet TKO mouse embryonic stem cells (mESC) (data from (25)). Only windows with 3 or more CpGs per 1 kb, or 10 or more CpGs per 10 kb, covered by at least 5 WGBS reads per CpG, were considered for the analysis. Two-sample Kolmogorov-Smirnov test comparing the distributions of WT and Tet TKO was used to calculate the Dₖₛ statistic and p-value. (Right panels) Correlation between DNA methylation changes (difference in cytosine modification percentage, mutant minus wild type) and euchromatin/heterochromatin compartments (positive versus negative Hi-C PC1 values), in Tet TKO (top), and Dnmt TKO (bottom) mESC. Spearman correlation coefficient is shown (rₛ value). (FIG. 1B), Density distribution (left panel) and cumulative distribution (middle panel) of average DNA methylation values within 10 kb windows across the genome in wild type (WT) and Tet TKO mouse embryonic stem cells (mESC) (data from (15)). (Right panel) Correlation between DNA methylation changes and euchromatin/heterochromatin compartments. Spearman correlation coefficient is shown (rₛ value). (FIG. 1C), Relationship between 5hmC distribution (CMS-IP) and euchromatin/heterochromatin compartments (Hi-C PC1 values) in WT mESC, HSC and pro-B cells. CMS-IP enrichment was calculated for 1 kb windows. (FIG. 1D), Density distribution (left panel) and cumulative distribution (middle panel) of average DNA methylation values within 10 kb windows across the genome, in wild type (WT), Tet1 KO, and Tet2 KO mESC (data from (12)). (Right panel) Correlation between DNA methylation changes and euchromatin/heterochromatin compartments. Spearman correlation coefficient is shown (rₛ value).

FIG. 2. DNA hypomethylation in heterochromatin compartment in TET-deficient mouse ESC. Genome tracks showing an overlap between Hi-C-defined A/B compartments (track 1), early/late replicating sites (early replicating: positive values; track 2), lamina-associated domains (track 3), regions marked by histone marks H3K9me2 (track 4) and H3K27me3 (track 5), and large hypomethylated domains in different Tet KO mESC (shown as subtraction of DNA methylation percentage, mutant minus wild type; tracks 6-9). TET1 and TET2 binding (ChIP-seq; tracks 10-11), 5hmC distribution (TAB-seq and CMS-IP; tracks 12-13) and gene expression (RNA-seq; tracks 14) in WT mESC are shown for reference. Heterochromatic Hi-C B compartment regions are highlighted.

FIGS. 3A-3F. DNA hypermethylation in euchromatin and hypomethylation in heterochromatin in NKT cell lymphoma from Tet2/3 DKO mice. (FIG. 3A, 3B) Cumulative (FIG. 1A) and density distribution (FIG. 3B) of average DNA methylation values within 1 kb and 10 kb windows across the genome, in wild type (WT), young, and transferred and expanded Tet2/3 DKO NKT cells. In (3A), two-sample Kolmogorov-Smirnov test comparing the distributions of WT and Tet2/3 DKO (young or expanded, as indicated) was used to calculate the Dₖₛ statistic and p-value. (FIG. 3C), Correlation between DNA methylation changes (mutant minus wild type) and euchromatin/heterochromatin compartments in Tet2/3 DKO NKT cells at two stages of expansion (young, and transferred and expanded). Spearman correlation coefficient is shown (rₛ, value). (FIG. 3D), Correlation between changes in chromatin accessibility (ATAC-seq) in WT versus Tet2/3 DKO cells, and euchromatin/heterochromatin compartments, in thymic NKT cells from young mice. Accessibility differences were calculated at 1 kb resolution. (FIG. 3E), Correlation between 5hmC distribution (CMS-IP) and euchromatin/heterochromatin compartments in WT thymic NKT cells. CMS-IP enrichment was calculated for 1 kb windows. (FIG. 3F), Genome tracks showing a correspondence between Hi-C-defined B compartments in thymic and splenic WT NKT cells (tracks 1-2) and expanded splenic Tet2/3 DKO NKT cells (track 3), and large hypomethylated domains in WT, young and expanded Tet2/3 DKO NKT cells (tracks 4-8). Notice progressive hypomethylation of the heterochromatic compartment. 5hmC distribution (CMS-IP, track 9), chromatin accessibility (ATAC-seq, track 10), and gene expression (RNA-seq, track 11) in WT NKT cells are shown to contextualize methylation changes. The heterochromatic regions that lose DNA methylation in Tet2/3 DKO T cells are highlighted.

FIGS. 4A-4E. Transposable element reactivation and increased mutations predominate in heterochromatin of TET-deficient NKT cell lymphoma. (FIG. 4A), Genome-wide distribution (top), and percentage of SNVs (bottom) located within euchromatin and heterochromatin compartments in Tet2/3 DKO expanded NKT cells. (FIG. 4B), Coding mutations resulting in change in the amino acid sequence in five independent WGS samples from Tet2/3 DKO expanded NKT cells. Mutant allele frequencies are shown in parentheses. (FIG. 4C), Distribution of the changes in expression (log₂ fold change) of transposable elements (TEs) belonging to the LTR, LINE and SINE families in Tet2/3 DKO young NKT cells compared to WT, obtained from analysis of total RNA-seq data. Fold change differences for all genes in the genome are shown for reference. (FIG. 4D), Volcano plots of expression of TEs belonging to the LTR, LINE and SINE families, in Tet2/3 DKO young NKT cells compared to WT. Differentially expressed (DE) TEs (adj. p-value<0.1) are highlighted. (FIG. 4E), Percentage of SNVs in Tet2/3 DKO expanded NKT cells that overlap with LINEs, LTR, and SINEs, compared to the mm10 genome distribution of each TE families p-values were calculated by one sample t-test.

FIGS. 5A-5D. Increased DNA hypomethylation in double DNA/11′3A and TET2 mutant mice. (FIG. 5A), Correlation between DNA methylation changes (mutant minus wild type) and euchromatin/heterochromatin compartments, in Tet2 KO (top), Dnmt3a KO (middle), and Dnmt3a/Tet2 DKO (bottom) hematopoietic stem cells (HSPC). Spearman correlation coefficient is shown (rₛ value). DNA methylation data from (27, 46). (FIG. 5B), Genome tracks showing a correspondence between the Hi-C B compartment (negative Hi-C PC1 values, track 1) and large hypomethylated domains in Tet2 KO (track 2). In contrast, Dnmt3a KO and Dnmt3a/Tet2 DKO show a global, compartment-independent DNA hypomethylation (tracks 3 and 4). Gene expression (track 5) and 5hmC distribution (track 6) in WT HSPC are shown for reference. (FIG. 5C, 5D), Density (FIG. 5C) and cumulative distribution (FIG. 5D) of average DNA methylation values within 1 kb (left) and 10 kb (right) windows across the genome, in wild type (WT), Dnmt3a KO, Tet2 KO, and Dnmt3a/Tet2 DKO HSPC.

FIGS. 6A-6C. Contributions of DNMT3 enzymes to DNA methylation in euchromatin and heterochromatin. (FIG. 6A), Genome-wide distribution of the de novo DNA methyltransferases DNMT3A1, DNMT3A2 and DNMT3B1 within euchromatin and heterochromatin compartments in WT mESC. DNMT ChIP-seq enrichment (log₂ fold change) was calculated at 1 kb resolution. Data from (51, 52). (FIG. 6B), Genome tracks showing the distribution of DNMT3A1, DNMT3A2 and DNMT3B1 (tracks 2-4) within Hi-C defined compartments (track 1), and the contribution to DNA methylation of the de novo DNMT proteins after reconstitution in a Dnmt TKO background in mESC (tracks 5-7). Dnmt TKO methylation is shown for reference (track 8). (FIG. 6C), Density distribution of average DNA methylation values within 10 kb windows across the euchromatin and heterochromatin compartments, in Dnmt TKO mESC reconstituted with DNMT3A1, DNMT3A2, and DNMT3B1.

FIGS. 7A-7F. Relocalization of DNMT3A away from the heterochromatin compartment of TET-deficient mESC. (FIG. 7A), Comparison of changes in DNMT3A1 binding in Tet1 KO versus WT mESC, and euchromatin versus heterochromatin compartments (Hi-C PC1 values) in WT mESC. Differential binding was calculated at 1 kb windows and displayed are those with p-value<0.05. Percentage of windows within each quadrant is indicated. (FIG. 7B), Genome tracks showing a correspondence between the heterochromatic Hi-C B compartment (track 1) and decrease in DNMT3A1 occupancy in Tet1 KO with respect to WT (tracks 2-4). Tracks 2 and 3 show DNMT3A1 occupancy in WT and Tet1 KO mESC, respectively (shown as reads per kilobase per million reads). Track 4 shows the difference in DNMT3A1 occupancy (visualized as log₂ fold-change Tet1 KO/WT). A region containing the Lefty1/2 locus is highlighted by the red arrowhead and is shown in (FIG. 7C). (FIG. 7C), Zoomed-in view of the Lefty1/2 locus within the euchromatic compartment. Genome tracks showing mutually exclusive localization of TET1 and TET2 (tracks 1-6) and de novo DNMT proteins (tracks 7-10). (FIG. 7D), Mutually exclusive localization between DNMT3A1 and TET1 binding in the euchromatic Hi-C A compartment in WT mESC. Percentage of windows within each quadrant is indicated. ChIP-seq enrichment (log₂ fold change) was calculated for 1 kb windows. DNMT3A1 data from (53); TET1 data from (54). (FIG. 7E), Distribution of DNMT3A1 occupancy in WT and Tet1 KO mESC (displayed as enrichment over input) within each of the quadrants defined in (FIG. 7D). (FIG. 7F), Schematic model illustrating that loss of TET1 in mouse ES cells results in the relocalization of DNMT3A1, on the one hand from the heterochromatic to the euchromatic compartment as shown in FIG. 7B, and on the other hand to regions within euchromatin that were previously occupied by TET1 as shown in FIG. 7D, 7E.

FIGS. 8A-8C shows the B compartment hypomethylation in TET-mutant mESC. Left: Smoothed scatterplot of the average DNA methylation values within 1 kb windows across the genome, comparing WT cytosine modification values (x-axis) to the ones in Tet TKO (upper), and Dnmt TKO (lower) (y-axis). LOESS regression (dashed line) is displayed for each panel. Middle: Smoothed scatterplot of the average DNA methylation values within 1 kb windows across the genome, comparing WT cytosine modification values (x-axis) to the ones in Tet TKO (y-axis). LOESS regression (dashed line) is displayed for each panel. Right: Smoothed scatterplot of the average DNA methylation within 1 kb windows across the genome, com-paring cytosine modification values between WT (x-axis) and Tet1 KO or Tet2 KO (y-axis) mESC. LOESS regression (dashed line) is displayed for each panel. Tet1 KO mESC (upper) show global loss of methylation whereas Tet2 KO mESC (lower) show hypermethylation in regions with low-intermediate methylation in WT mESC and hypomethylation at regions with high methylation in WT mESC.

FIGS. 9A-9E. B compartment hypomethylation in TET-mutant NPC and pro-B cells. (9A), Distribution of average DNA methylation values within 1 kb and 10 kb windows across the genome in wild type (WT) and Tet2 KO mESC differentiated to neural precursor cells (NPC) at day 3 post-differentiation. (9B), Correlation between DNA methylation changes (difference in cytosine modification percentage, Tet2 KO minus WT) and A/B compartments (Hi-C PC1 values) in mESC differentiated to NPCs. The Spearman correlation coefficient is shown (rs value). (9C), Distribution of average DNA methylation values within 1 kb and 10 kb windows across the genome, in wild type (WT) and Tet2/3 DKO pro-B cells. (9D), Correlation between DNA methylation changes (difference in cytosine modification percentage, Tet2/3 DKO minus WT) and A/B compartments (Hi-C PC1 values) in mouse pro-B cells. The Spearman correlation coefficient is shown (rs value). (9E), Genome tracks showing a correspondence between heterochromatic Hi-C B compartment and large hypomethylated domains in pro-B cells (shown as subtraction of DNA methylation percentage, Tet2/3 DKO minus WT). RNA transcription (GRO-seq track) and 5hmC distribution (CMS-IP track) in WT pro-B cells are shown for reference.

FIGS. 10A-10G. Expansion of Tet2/3 DKO NKT cells is accompanied by increased clonality, and accumulation of DNA double-strand breaks and R-loops. (10A), Left, experimental workflow. Middle, the picture depicts splenomegaly and enlarged lymph nodes in wild type (WT) but not CD1d KO recipients of Tet2/3 DKO NKT cells. Right, times of disease emergence (see (B)). (10B), Percentage of WT or expanded Tet2/3 DKO NKT cells in splenocytes of congenic WT recipient mice, injected with the indicated numbers of Tet2/3 DKO NKT cells. 2 mice were used per condition. Mice injected with as few as 50 Tet2/3 DKO cells develop NKT cell lymphoma. (10C), Evaluation of TCRβ chain CDR3 variable region sequences in DNA from NKT cells. One WT mouse, five 3-4 week old Tet2/3 DKO mice, and seven WT recipients of 0.5 million Tet2/3 DKO NKT cells were evaluated. Each color represents a single TCR Vβ sequence. Mice that received NKT cells from a single Tet2/3 DKO donor showed expansion of the same NKT cell clone. Asterisks indicate independent mice Mouse D and Mouse E for which SNV data are shown in FIG. 4B. (10D), Evaluation of phospho-H2AX staining as a marker for DNA DSBs in WT and Tet2/3 DKO NKT cells. NKT cells (aGalCer-CD1d+, TCRβ+) were isolated from healthy WT mice or after transfer of Tet2/3 DKO NKT cells to non-irradiated WT or CD1d KO recipients as outlined in (10A). A representative flow cytometric analysis is shown. (10E), (Top panel) Percentage of phospho-H2AX+ NKT cells isolated from WT mice or from Tet2/3 DKO NKT cells transferred to and recovered from non-irradiated WT or CD1d KO recipients as outlined in (10A). (Bottom panel) Median fluorescence intensity of phospho-H2AX staining in NKT cells isolated from WT or from Tet2/3 DKO mice after transfer to and recovery from WT or CD1d KO recipients as outlined in (10A). Data are mean±SEM, n=2 (WT NKT cells), n=3 (NKT cells from CD1d KO recipients) and n=4 (NKT cells from WT recipients). ns, not significant. At least 2 independent experiments were performed per condition. (10F), Flow cytometric analysis evaluating R-loops in NKT cells isolated from healthy WT mice (blue histogram) or from Tet2/3 DKO NKT cells transferred and expanded in nonirradiated immunocompetent WT recipients (red histogram). The S9.6 antibody recognizes RNA:DNA hybrids. (10G), Dot blot of genomic DNA from Tet2/3 DKO NKT cells transferred to and expanded in non-irradiated WT recipients shows increased R-loop formation compared to WT NKT cells (top panel, right). Specificity for RNA:DNA hybrids was confirmed by RNase H treatment of genomic DNA prior to spotting, which results in elimination of the signal (top panel, left). Equivalent DNA loading was confirmed by methylene blue staining (bottom panel).

FIGS. 11A-11D. Tet2/3-deficient NKT cell lymphoma displays progressive hypomethylation in the heterochromatic Hi-C B compartment. (11A), Smoothed scatterplot of the average DNA methylation within 1 kb windows across the genome, comparing WT (x-axis) to young (left panel) or transferred and expanded (right panel) Tet2/3 DKO NKT cells (y-axis). LOESS regression (dashed line) is displayed for each panel. (11B), Distribution of DNA methylation (WGBS signal) at cytosines within the CpG context covered by at least 5 WGBS reads. DNA methylation values are shown for WT, Tet2/3 DKO young, and expanded Tet2/3 DKO NKT cells. (11C), Comparison of gene expression levels (RNA-seq, log 2(TPM+1)) in the euchromatic Hi-C A and heterochromatic Hi-C B compartments in young (left panel) and expanded (right panel) Tet2/3 DKO NKT cells. (11D), Pairwise comparison of Hi-C PC1 values between independent samples. The correlation between the WT thymus and spleen NKT cell samples and between Tet2/3 DKO replicates 1 and 2 which were expanded from the same donor mouse, compared to the slightly greater differences between Tet2/3 DKO replicates 1 and 2 and replicate 3 which was from a different donor mouse. Similarly, there are slight differences in the Hi-C compartment between NKT cells from WT spleen and all three Tet2/3 DKO NKT cells taken from the spleen.

FIGS. 12A-12E. Mutational spectrum of transferred and expanded Tet2/3 DKO T cells. (12A), Mutational spectrum associated with euchromatic Hi-C A and heterochromatic Hi-C B compartments in Tet2/3 DKO expanded NKT cells. (12B), Cosine similarity between mutational profiles obtained from three independent Tet2/3 DKO expanded T cells WGS samples (matched tail samples, Mouse A-C), separating the mutations by their location within Hi-C compartments (A vs B) in Tet2/3 DKO NKT cells. Notice how mutational profiles cluster by Hi-C compartment and not by sample of origin. (12C), DNA methylation at cytosines within the CpG context with C>T substitution type. DNA methylation values shown for WT, Tet2/3 DKO young, and Tet2/3 DKO expanded T cells. (12D), Rainfall plots representing the inter-mutational genomic distance (y-axis) for all single nucleotide variants (SNV) (x-axis) encountered in samples Mouse A-C. Substitutions are color-coded as indicated in the top of the figure. While mutations cluster at certain regions, each mice exhibits a unique spectrum of mutations. (12E), Distribution of the changes in expression (log 2 fold change difference) of LTR and LINE transposable elements in Tet2/3 DKO young NKT cells compared to WT, obtained from analysis of polyA+ RNA-seq data. P-values of two independent experiments (same biological conditions, different library preparation methods, TruSeq and SMARTseq) were combined using the Fisher method. (12F), Table with coverage values of WGS samples.

FIGS. 13A-13C. TET TKO mESC proliferate more slowly than their WT counterparts, and genome-wide distribution of DNMT3 enzymes in relation to TET1 and TET2 in mESC. (13A), Growth curves of WT and Tet1/2/3 TKO mESCs. Cells were split every 3 days, and cells were counted. Reprinted from ref 16. (13B-13C), Comparison of the localization of DNMT3 proteins versus TET1 (13B) and TET2 (13C) in WT mESC. ChIP-seq enrichment (log 2 fold change) was calculated for 1 kb windows. A different dataset (51, 52) was used from that of FIG. 7.

FIG. 14. Role of TET enzymes in DNA demethylation. Unmodified cytosines in DNA are methylated by DNA methyltransferases (DNMT) to yield 5mC. TET proteins successively oxidize 5mC to the oxidized methylcytosines (oxi-mC) 5hmC, 5fC and 5caC. Oxi-mCs on the unreplicated DNA strand in the CpG sequence context are not recognized by the DNMT1/UHRF1 complex, which recognizes hemi-methylated CpGs; this prevents restoration of symmetrical methylation on the newly-replicated strand and facilitates “passive” (replication-dependent) DNA demethylation (top arrow). TET proteins can also facilitate DNA demethylation independently of DNA replication (bottom arrow): SIC and 5caC can both be excised by thymine DNA glycosylase (TDG) and replaced with unmodified cytosine through base excision repair (BER).

FIG. 15. The euchromatic (Hi-C A) and heterochromatic (Hi-C B) compartments of the genome: Genomic features, replication timing and nuclear positioning.

FIGS. 16A-16B. Profound TET deficiency is causative for cancer. 16A, Flow chart of experiment. Tet2/3 fl/fl mice carrying an inducible Cre (Mx1Cre or Cre-ERT2) were injected 5 times with polyI:polyC or tamoxifen as indicated. 1 6B, Kaplan-Meier curve of survival shows that the mice succumb between 4 and 6 weeks of injection. C, Massive splenomegaly, anemia and a dramatic drop in frequencies of erythrocyte precursors in the bone marrow by 4 weeks. The overall outcome is skewed hematopoietic differentiation with massive myeloid expansion and extreme reduction in B cells, T cells, platelets and erythrocytes within 4 weeks of acute Tet2/3 gene deletion.

FIG. 17. Increased self-renewal and increased clonality of inductibly Tet1/2/3-deficient HSPC.

FIG. 18. WGBS and Hi-C of Tet2/3 DKO T cells show hypermethylation in the active fraction of the genome, but progressive hypomethylation in the inactive fraction of the genome. Tracks 1-3: There are only minor changes in the euchromatic (Hi-C A, pink) and heterochromatic (Hi-C B, blue) compartments of wildtype (WT) spleen versus WT thymus (tracks 1, 2), and WT versus Tet2/3 DKO spleen (tracks 2, 3). Tracks 3-6: DNA methylation increases in euchromatin but decreases progressively in heterochromatin of young and expanded Tet2/3 DKO NKT cells compared to WT cells. Tracks 7, 8: Differences between Tet2/3 DKO and WT NKT cells are shown; black values above the line indicate increased methylation whereas grey values below the line indicate demethylation. Tracks 9-13: Genes, transcribed and accessible genomic regions and 5hmC are all predominantly in euchromatin.

FIGS. 19A-19C. G-quads in WT and Tet-deficient mouse B and myeloid cells (19A, 19B) and mouse myeloid cells (19C) detected with the G-quad-binding dye NMM. A, Mice in which the Tet2 and Tet1 genes have been deleted in mature B cells using CD19Cre develop B cell lymphomas by 10 weeks and succumb by 20 weeks of age. B, Tet2/3 DKO B cells and Tet1/2/3 TKO myeloid cells (similar to the Tet2/3 DKO myeloid cells shown in FIG. 6A) show increased staining with NMM compared to the corresponding control cells.

FIG. 20. Proposed mechanism for loss of heterochromatic DNA methylation in TET-deficient cells: Relocalisation of DNMT3A to the locations in euchromatin where TET typically binds.

FIGS. 21A-21C. Biological validation of the mechanism of DNMT relocalisation proposed in FIG. 20: if movement of DNMT3A away from heterochromatin is responsible for the increased self-renewal and oncogenic transformation observed in TET-deficient cells, targeting DNMT3A to heterochromatin might be expected to rescue these features. The inventors have found that restoration of DNMT function, specifically DNMT3A, in heterochromatin has potentially therapeutic effects.

FIG. 22. Metabolic regulation of TET enzymatic activity. Left, the TCA cycle intermediate isocitrate is converted into the TET co-substrate α-ketoglutarate (αKG) (middle) by cytoplasmic and mitochondrial isocitrate dehydrogenases IDH1 and IDH2 respectively. 2-hydroxyglutarate (2HG), a metabolite structurally similar to αKG, inhibits TET activity. 2HG has two stereo-isomers: the potent dioxygenase inhibitor L-2HG, also known as S-2HG (top), and the less potent inhibitor D-2HG, also known as R-2HG (bottom) (also see FIGS. 5A-5D). The dehydrogenases L2HGDH and D2HGDH convert these metabolites back to αKG; loss-of-function mutations of these enzymes result in increased levels of L-2HG and D-2HG respectively, while recurrent gain-of-function mutations of IDH1/2 generate D-2HG. Overexpression of lactate dehydrogenase A (LDHA) and malate dehydrogenases (MDH1/MDH2) also generates L-2HG. Right, BCAT1 (branched-chain amino acid transaminase 1) reversibly transfers the amino group from the branched-chain amino acids (BCAA) leucine, isoleucine, and valine to αKG, to yield branched-chain a-keto acids (BCKA) and glutamate. High levels of BCAT1 result in decreased αKG levels, thus interfering with αKG-dependent enzymes including TET proteins.

FIGS. 23A-23B. FIG. 23A, 23B, Recurrent aneuploidy of chromosome 17, and partial recurrent aneuploidy of Chromosome 2, in transferred and expanded Tet2/3 DKO NKT cells. FIG. 23C, result of single-cell, low-coverage whole-genome sequencing (WGS) on NKT cells from a wildtype mouse.

FIGS. 24A-24C. Acute deletion of all three TET genes is associated with increased chromosome segregation defects and aneuploidies. The cells showed a major loss of 5hmC by flow cytometry. 24A, Mouse genotypes and methods used to derive TET iTKO mESC. 24B, Representative chromosomal segregation defects observed by live cell imaging in ES cells. Note lagging chromosomes in all panels and micronuclei in i-k). 24C, aneuploidies measured by metaphase spreads.

FIG. 25. Dnmt1 or Dnmt3a deficiency in mouse hematopoietic cells is accompanied by copy number alterations (CNAs), aneuploidies and DNA damage. Recurrent chromosome 15 trisomy, other aneuploidies and CNAs in Dnmt1-hypomorphic mice.

FIG. 26. Dnmt1 or Dnmt3a deficiency in mouse hematopoietic cells is accompanied by copy number alterations (CNAs), aneuploidies and DNA damage. Chromosome 15 and 17 trisomies in CLL and PTCL developing in mice lacking Dnmt3a in hematopoietic stem/precursor cells (HSPC).

FIG. 27. Mice receiving doubly Dnmt3a/Tet2-deficient HSPC show more severe phenotypes than mice singly deficient in either Dnmt3a or Tet2 alone. Numbers of circulating monocytes, frequencies of LSK precursor cells, numbers of colonies obtained in serial replating assays, decrease in numbers of erythroid precursors, and overall survival curves were assessed.

FIG. 28. Mice receiving doubly Dnmt3a/Tet2-deficient HSPC show more severe phenotypes than mice singly deficient in either Dnmt3a or Tet2 alone. Numbers of circulating monocytes, frequencies of LSK precursor cells, numbers of colonies obtained in serial replating assays and decrease in numbers of erythroid precursors were assessed.

FIG. 29. Mice receiving doubly Dnmt3a/Tet2-deficient HSPC show more severe phenotypes than mice singly deficient in either Dnmt3a or Tet2 alone; overall survival curves were assessed.

FIG. 30. 5hmC is most highly enriched in the gene bodies of the most highly expressed genes (upper panel) and at the most active enhancers (lower panel).

FIG. 31. Two TET-regulated enhancers in the Aicda locus, TetE1 and TetE2, show progressive 5hmC enrichment during B cell activation for class switch recombination.

FIG. 32. Volcano plots of expression of transposable elements reveals that expanded, TET-deficient NKT and myeloid cells show increased expression of transposable and repeat elements, particularly LINEs and LTRs primarily located in heterochromatin.

FIG. 33. Transposable element reactivation and increased mutations predominate in heterochromatin of TET-deficient NKT-cell lymphoma. Genome-wide distribution (upper) and percentage of SNVs (bottom) located within euchromatin and heterochromatin compartments in Tet2/3 DKO expanded NKT cells. RNA sequencing reveals that expanded TET-deficient NKT cells display accumulation of single nucleotide polymorphisms preferentially in the heterochromatic compartment.

FIG. 34. Genome-wide views of methylation levels across chromo-some 16 in human CD4 T cells from a newborn (top), from a 103-year old individual (middle) and from a patient with prolymphocytic T cell leukemia (bottom). The blue regions are hypomethylated and correspond to heterochromatic regions such as pericentromeres. Note the similar patterns of DNA hypomethylation in heterochroma-tin in old and leukemic cells.

FIG. 35. TET enzymes mediate—not just facilitate—passive DNA demethylation.

FIGS. 36A-36B. 36A, Definition of solo-WCGW CpGs. FIG. 36B, Solo-WCGW CpGs in partially methylated domains (PMDs) in heterochromatin show the greatest losses of DNA methylation, both in normal colon and in colorectal cancers (CRC).

FIG. 37. Tet2/3-deficient NKT cells display pronounced DNA demethylation in the WGCW solo-CpG sequence context. Top two tracks, Hi-C PC1 values (also see FIG. 34) were used to divide the genomes of NKT cells from WT spleen and thymus into euchromatic (Hi-C A, positive PC1 values, pink) and heterochromatic (Hi-C B, negative PC1 values, blue) compartments. Bottom four tracks, The data from track 8 of FIG. 34 (transferred and expanded Tet2/3 DKO NKT cells) are shown for CpGs in the WCGW sequence context. The highest loss of DNA methylation occurs in the WCGW sequence context with the lowest CpG density (see FIGS. 36A-36B).

FIG. 38. Proposed mechanism for loss of heterochromatic DNA methylation in TET-deficient cells: Relocalization of DNMT3A to the locations in euchromatin where TET typically binds.

FIG. 39. Metabolic regulation of TET enzymatic activity. Left, the TCA cycle intermediate isocitrate is converted into the TET co-substrate α-ketoglutarate (αKG) (middle) by cytoplasmic and mitochondrial isocitrate dehydrogenases IDH1 and IDH2 respectively. 2-hydroxyglutarate (2HG), a metabolite structurally similar to αKG, inhibits TET activity. 2HG has two stereoisomers: the potent dioxygenase inhibitor L-2HG, also known as S-2HG (top), and the less potent inhibitor D-2HG, also known as R-2HG (bottom) (also see FIG. 35). The dehydrogenases L2HGDH and D2HGDH convert these metabolites back to αKG; loss-of-function mutations of these enzymes result in increased levels of L-2HG and D-2HG respectively, while recurrent gain-of-function mutations of IDH1/2 generate increased D-2HG. Overexpression of lactate dehydrogenase A (LDHA) and malate dehydrogenases (MDH1/MDH2) also generates L-2HG. Right, BCAT1 (branched-chain amino acid transaminase 1) reversibly transfers the amino group from the branched-chain amino acids (BCAA) leucine, isoleucine, and valine to αKG, to yield branched-chain a-keto acids (BCKA) and glutamate. High levels of BCAT1 result in decreased αKG levels, thus interfering with αKG-dependent enzymes including TET proteins.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Cancer genomes are characterized by focal increases in DNA methylation, co-occurring with widespread hypomethylation. The inventors have shown that TET deficiency in diverse cell types (ESC, NPC, HSC, pro-B and T cells) results in a similar methylation landscape, with the expected localized increases in DNA methylation in active euchromatic regions, concurrently with unexpected losses of DNA methylation, reactivation of repeat elements and enrichment for single nucleotide alterations primarily in heterochromatic compartments. Thus, TET loss-of-function may be a primary mechanism underlying the characteristic pattern of global hypomethylation coupled to regional hypermethylation observed in diverse cancer genomes. This disclosure explains the synergy between DNMT3A and TET2 mutations in hematopoietic malignancies, as well as the recurrent association of TET loss-of-function with cancer.

TET enzymes are Fe(II) and α-ketoglutarate-dependent dioxygenases that mediate DNA demethylation through sequential oxidation of the methyl group of 5-methylcytosine (5mC) to 5-hydroxymethyl, 5-formyl and 5-carboxylcytosine (5hmC, 5fC and 5caC)(1-3). The oxidized methylcytosines (oxi-mC) generated by TET proteins are intermediates in at least two pathways of DNA demethylation: (i) replication-dependent loss of methylation, reflecting inability of the DNMT1/UHRF1 complex to methylate unmodified CpGs on newly replicated DNA strands if an oxi-mC (rather than 5mC) is present on the template strand, and (ii) a replication-independent process in which thymine DNA glycosylase (TDG) excises 5fC and 5caC, which are then replaced with unmodified cytosine through base excision repair (4).

Even in the absence of TET coding region mutations, TET loss-of-function and low 5hmC levels are strongly associated with cancer (5). TET2 mutations are frequent in diverse hematopoietic malignancies, including myelodysplastic syndromes (MDS), acute myeloid leukemias (AML) and peripheral T cell lymphomas (PTCL) (6-8). However, both solid tumors and hematopoietic malignancies display TET loss-of-function without TET coding region mutations, as a result of TET promoter methylation, increased degradation of TET proteins, or aberrant microRNA expression (9-11). In addition, hypoxia and a variety of metabolic alterations impair the enzymatic activity of TET and other dioxygenases, by decreasing the levels of the substrates α-ketoglutarate and molecular oxygen or increasing the levels of the inhibitor 2-hydroxyglutarate (10, 11).

Based on the known biochemical activities of TET-family proteins in oxidizing 5mC (4), TET loss-of-function mutations are expected to result in gains of DNA methylation. In fact, increased methylation as a result of TET loss-of-function has been documented at many genomic regions including promoters, enhancers and CTCF sites (9, 12-14).

Principal component analysis of the interaction matrix obtained from Hi-C data has been used to compartmentalize the genome into an A compartment (positive PCI values) and a B compartment (negative PC1 values) that exhibit the hallmark characteristics of euchromatin and heterochromatin, respectively (17, 18). The euchromatic A compartment is rich in expressed genes in the cell type under consideration, whereas the heterochromatic B compartment is gene-poor and bears epigenetically “repressive” chromatin marks, including H3K9me2/3 (17, 18). Moreover, the Hi-C B compartment overlaps with lamina-associated domains and corresponds to late-replicating regions of the genome, whereas the Hi-C A compartment corresponds to early replicating genomic regions and is not lamina-associated (18, 19). Notably, the extended, partially methylated domains (PMDs) observed in cancer genomes overlap with Hi-C B compartment, late-replicating, nuclear lamina-associated domains (20-22). The remainder of this disclosure will refer to the Hi-C A and B compartments as euchromatic and heterochromatic compartments, respectively.

Cancer genomes are characterized by two opposing patterns of aberrant DNA methylation: focal hypermethylation and widespread DNA hypomethylation (23). DNA hypermethylation at promoters and enhancers contributes to oncogenesis through transcriptional silencing of genes involved in DNA damage repair and tumor suppressors (23), and has been shown to reflect the impaired expression or activity of TET proteins. Despite understanding of the biochemical and biological consequences of local hypermethylation, however, the causes and consequences of DNA hypomethylation in cancer genomes are less well understood.

Here the inventors used a combination of Hi-C and WGBS data to document the DNA methylation changes associated with TET loss-of-function in diverse TET-deficient cell types. The inventors showed that 5hmC in wild type genomes, and DNA hypermethylation in TET-deficient genomes, are largely confined to the euchromatic Hi-C A compartment. This finding is consistent with the known functions of TET proteins in DNA demethylation and the known distribution of 5hmC at active enhancers and in the gene bodies of highly transcribed genes. In contrast, the inventors showed that the unexpected DNA hypomethylation noted in TET-deficient genomes is primarily present in the heterochromatic Hi-C B compartment. TET-deficient cells showed reactivation of repeat elements and pronounced enrichment for single-nucleotide variations (SNVs) in the heterochromatic Hi-C B compartment; this feature is characteristic of cancer genomes, in which mutation rates are elevated in genome compartments marked by H3K9me3 (24). The inventors also showed that DNMT3A relocalizes from the heterochromatic compartment to the euchromatic compartment in Tet1-deficient mESC, providing a mechanism for the heterochromatin hypomethylation observed in TET-deficient genomes. These results are consistent with the co-occurrence of DNMT3A and TET2 mutations in human cancers and the more pronounced leukemic phenotype observed in double Tet2/Dnmt3a-deficient mice compared to mice with individual disruption of Tet2 or Dnmt3a alone. Taken together, these data point to a functional interaction between TET proteins and DNMTs, and highlight the contribution of heterochromatic dysfunction to oncogenesis.

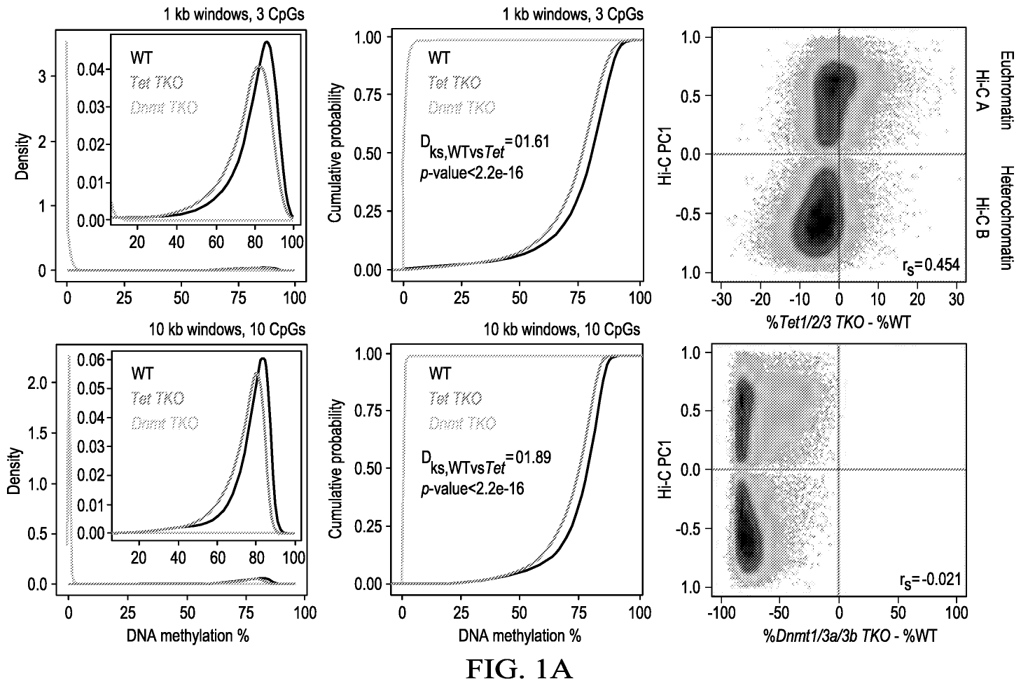

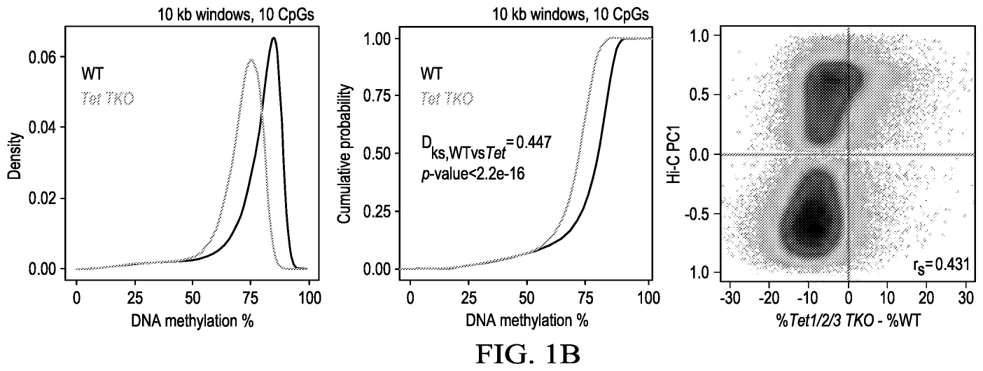

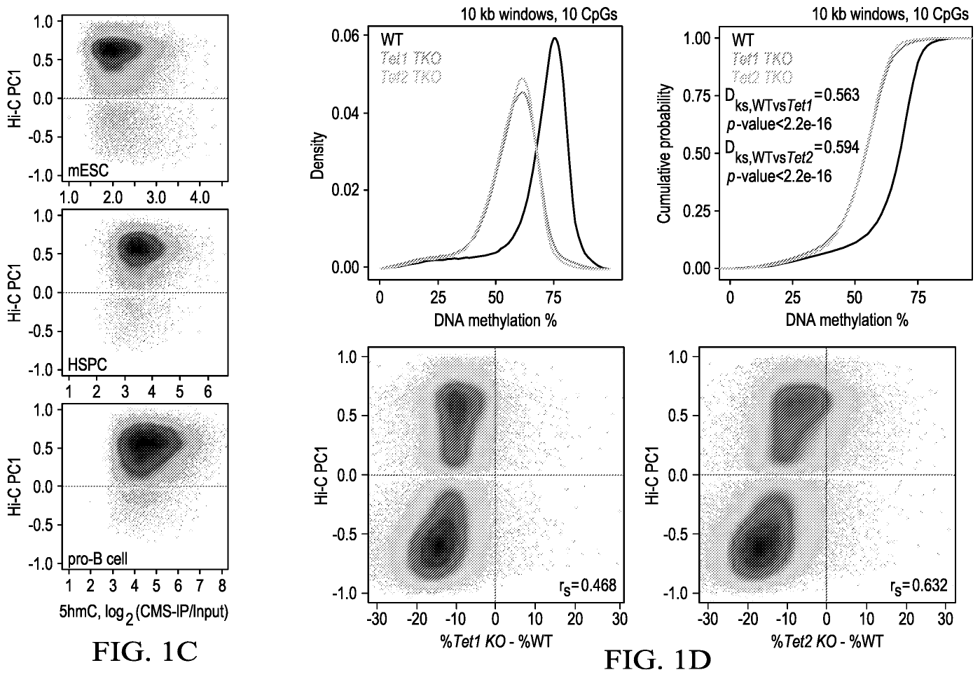

Widespread DNA hypomethylation in TET-deficient mouse ESC. To understand the impact of TET loss-of-function on genome-wide patterns of DNA modification, the inventors re-analyzed data from several publicly available WGBS datasets across a diverse range of TET-deficient murine cell types: ESC (12, 15, 25), neural precursor cells differentiated from ESC (NPC) (12); pro-B cells (26); hematopoietic stem/precursor cells (HSPC) (27) and a mouse model of TET-deficient T cells (14) (FIGS. 1-3, 5, 8, 9; and see below). Bisulfite sequencing estimates the sum of 5mC and 5hmC (28), but 5hmC is always <10% of 5mC in the control cell types considered here, and lower or absent in their TET-deficient counterparts. Thus, throughout this disclosure, the values obtained from WGBS are hereinafter referred to as “DNA methylation”.

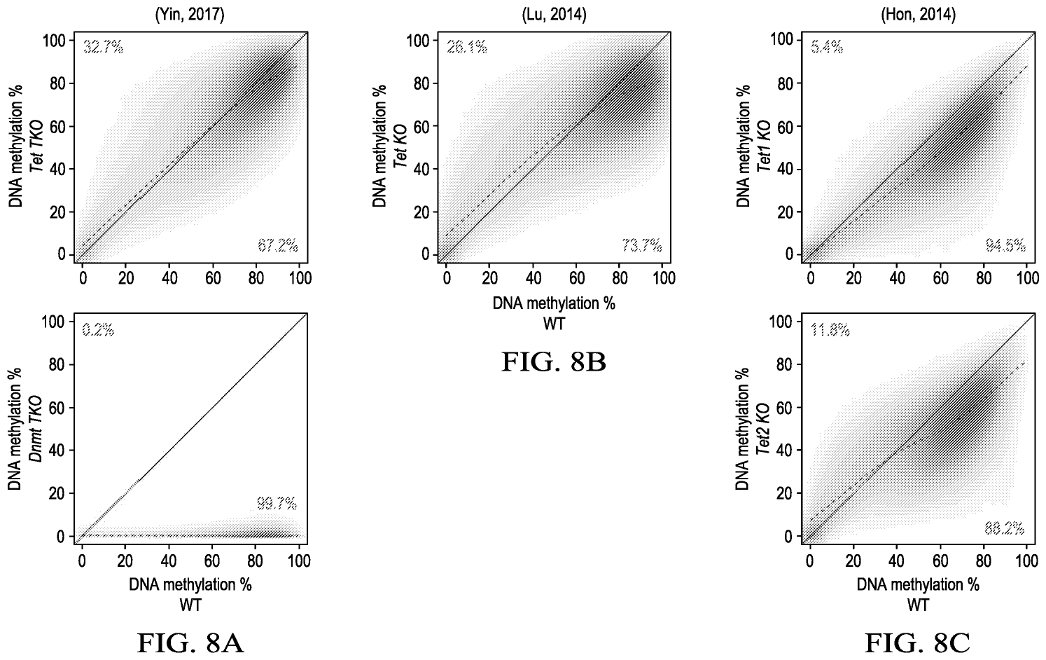

The inventors compared mouse embryonic stem cells (mESC) triply deficient in all three TET proteins (Tet1, Tet2 and Tet3; Tet TKO mESC) with mESC triply deficient in all three DNMTs (Dnmt1, Dnmt3a and Dnmt3b; Dnmt TKO mESC) (25) (FIG. 1A). The inventors plotted the distribution of average DNA methylation values in 1 and 10 kb windows containing at least 3 and at least 10 CpGs respectively (FIG. 1A). As expected, Dnmt TKO mESC lost all DNA methylation (5mC+5hmC; FIG. 1A, left and middle panels). Unexpectedly, however, Tet TKO mESC also showed pronounced and widespread loss of methylation compared to WT ESC, with the shift in the distribution of window methylation percentages visible at both 1 kb and 10 kb resolution (FIG. 1A, compare red and black traces). Hypomethylation in Tet TKO mESC could also be visualized in dot plots comparing DNA methylation levels in WT and mutant ESC (FIG. 8A). Each dot shows DNA methylation levels in 1 kb windows with at least 3 CpGs: 73.7% of the 1 kb windows showed decreased methylation in Tet TKO mESC compared to WT, whereas only 26.1% showed increased methylation (FIG. 8A). Analysis of WGBS data from an independent study of Tet TKO mESC, generated using CRISPR/Cas9 technology (15), yielded very similar results (FIG. 1B, FIG. 8B). The differences in global hypomethylation observed between WT and Tet TKO mESC in the two studies may be due to different culture conditions or different lengths of time that the cells were maintained in culture (compare FIGS. 1A, 1B).

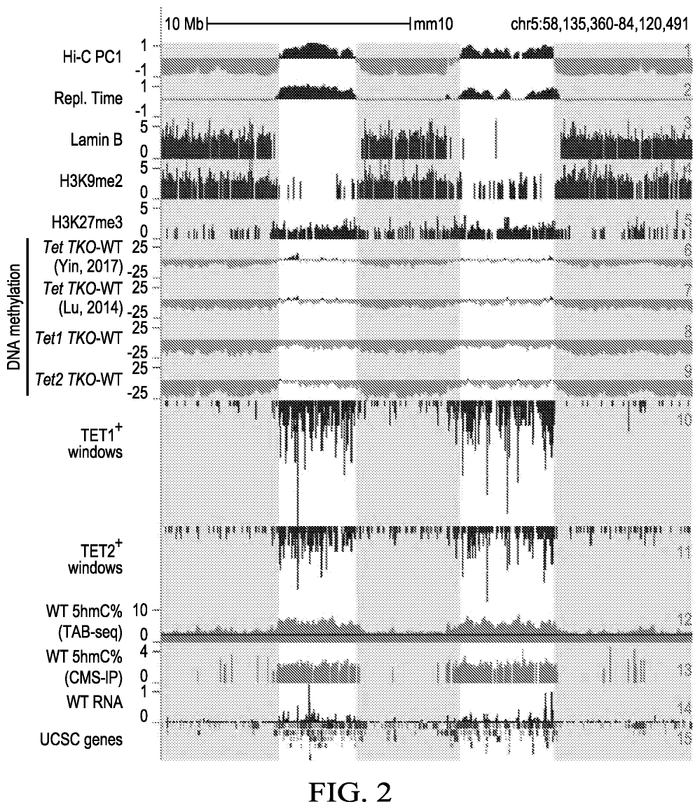

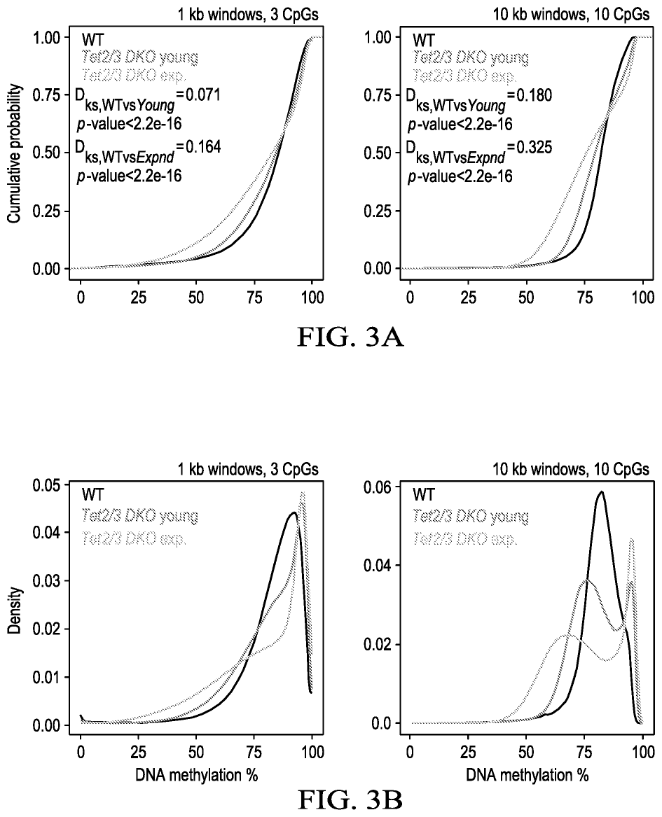

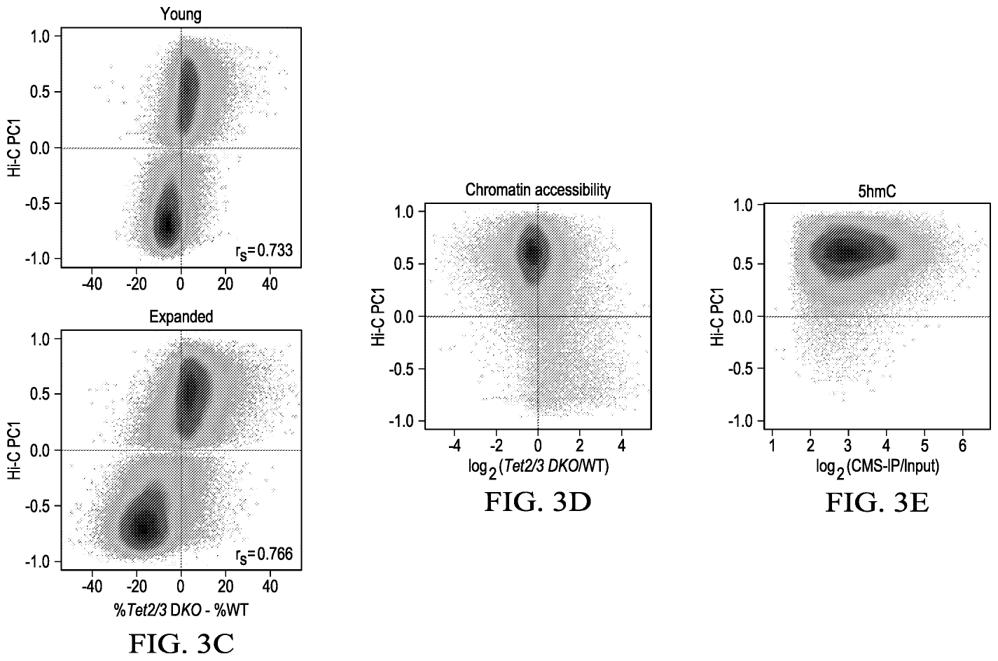

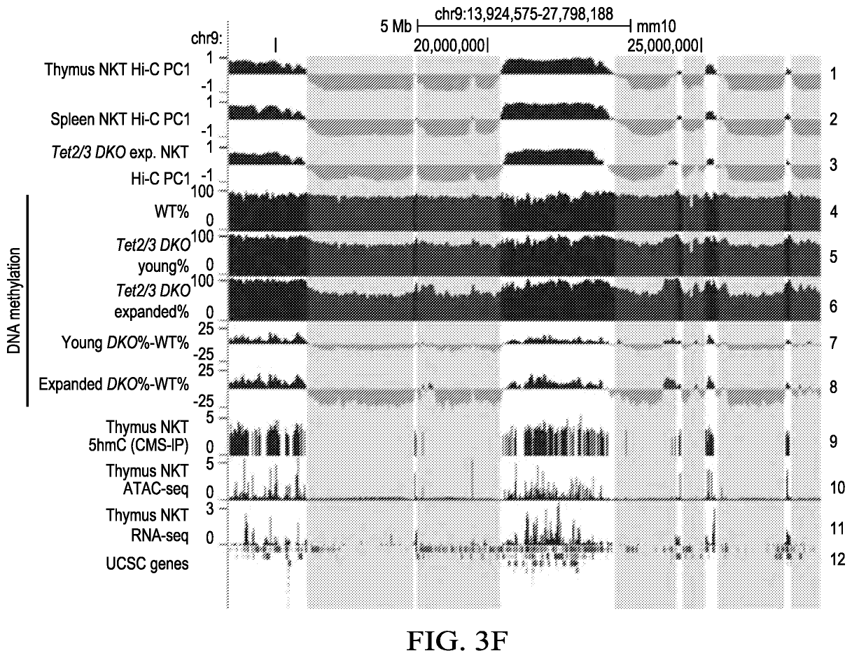

DNA hypermethylation in euchromatin and hypomethylation in heterochromatin in diverse TET-deficient cells. In genome browser views, DNA methylation changes were most striking when viewed at megabase-scale resolution (FIG. 2), and were reminiscent of the large, partially-methylated domains noted in cancer genomes (20, 21). Drawing on publicly available Hi-C (29), replication timing (19), Lamina B and H3K9me2 ChIP-seq data (30) on mESC, it was found that regions that lost DNA methylation in all Tet-mutant mESC (FIG. 2, tracks 6-9) mostly overlapped with the heterochromatic Hi-C B compartment (track 1), late replicating regions (track 2), lamina-associated domains (track 3), and regions marked by H3K9me2 (track 4). In contrast, H3K27me3 was present primarily in the euchromatic Hi-C A compartment (track 5). Likewise, TET1 and TET2 were primarily in the euchromatic compartment (tracks 10 and 11), consistent with the 5hmC distribution in WT mESC, as shown by two independent methods of mapping 5hmC: TAB-seq (12) and CMS-IP (31) (FIG. 2, tracks 12 and 13; the background signal for TAB-seq is noisier than for CMS-IP). The preferential presence of 5hmC in the euchromatic Hi-C A compartment was observed in all mouse cell types investigated, including ESC, HSC and pro-B cells (FIG. 1C; for similar data on mouse T cells please see FIG. 3E). The majority of hypermethylated genomic regions in TET-deficient cells are located in the euchromatic compartment; this finding, coupled with the presence of TET1, TET2 and 5hmC in the same euchromatic compartment in WT cells, confirms the by now well-established connection between DNA demethylation, TET-generated 5hmC, and 5hmC localization in actively transcribed genes and enhancers (4).

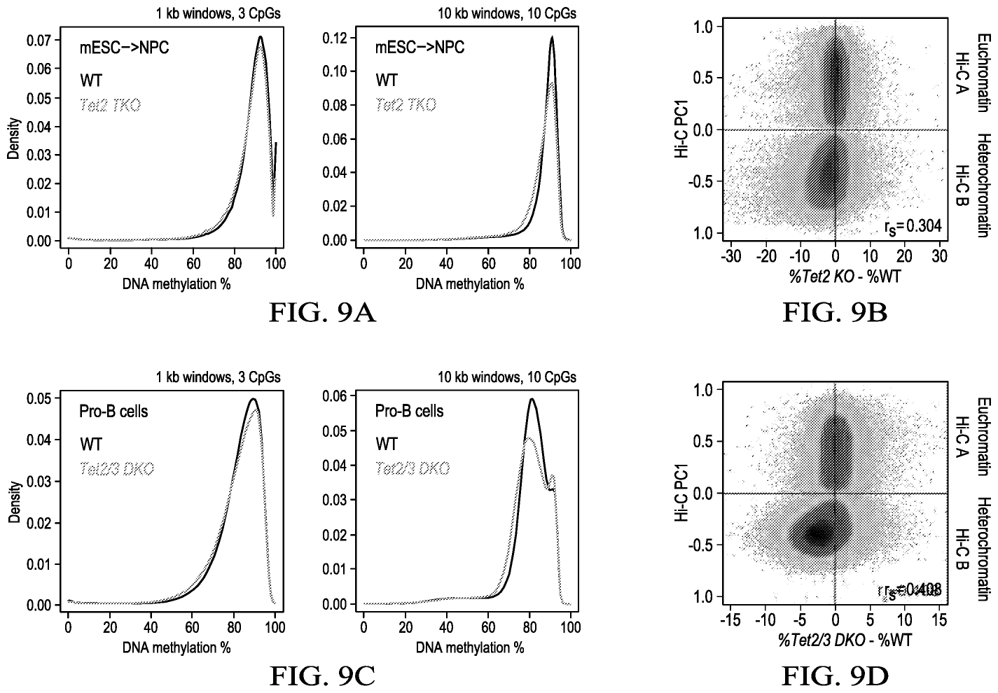

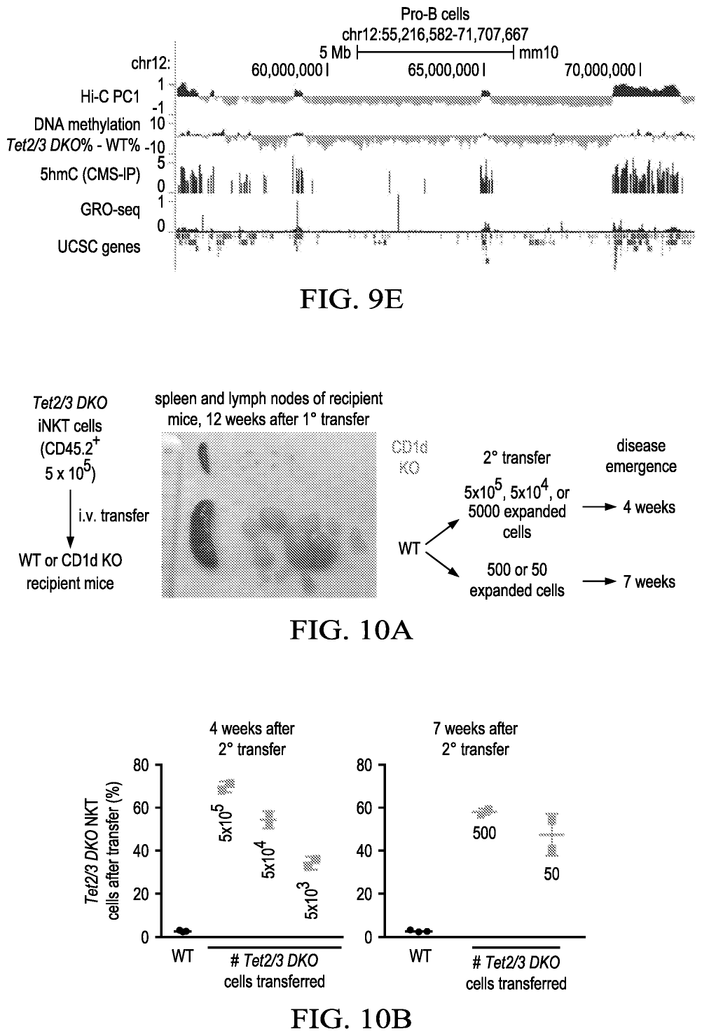

Dnmt TKO mESC showed genome-wide hypomethylation as expected (FIG. 1A, right panels), but the gains and losses of DNA methylation in Tet TKO mESC mostly occurred in the euchromatic and heterochromatic compartments respectively (FIG. 1A, 1B; FIG. 2, tracks 6-7; FIG. 8A, 8B). Even in singly-deficient Tet1 KO and Tet2 KO mESC (12), there is striking global DNA hypomethylation in both the euchromatic and heterochromatic compartments, most notably in the latter, which far exceeded the extent of the expected DNA hypermethylation (FIG. 1D; FIG. 2, tracks 8-9). Hypermethylated genomic regions were clearly apparent in Tet2 KO mESC, but there was only marginal hypermethylation in Tet1 KO mESC (12) (FIG. 1D, FIG. 8C). Even when Tet2 KO mESC were differentiated to neural precursor cells (NPC) for 3 days (12), the differentiated NPC showed heterochromatin hypomethylation (FIG. 9A, 9B).

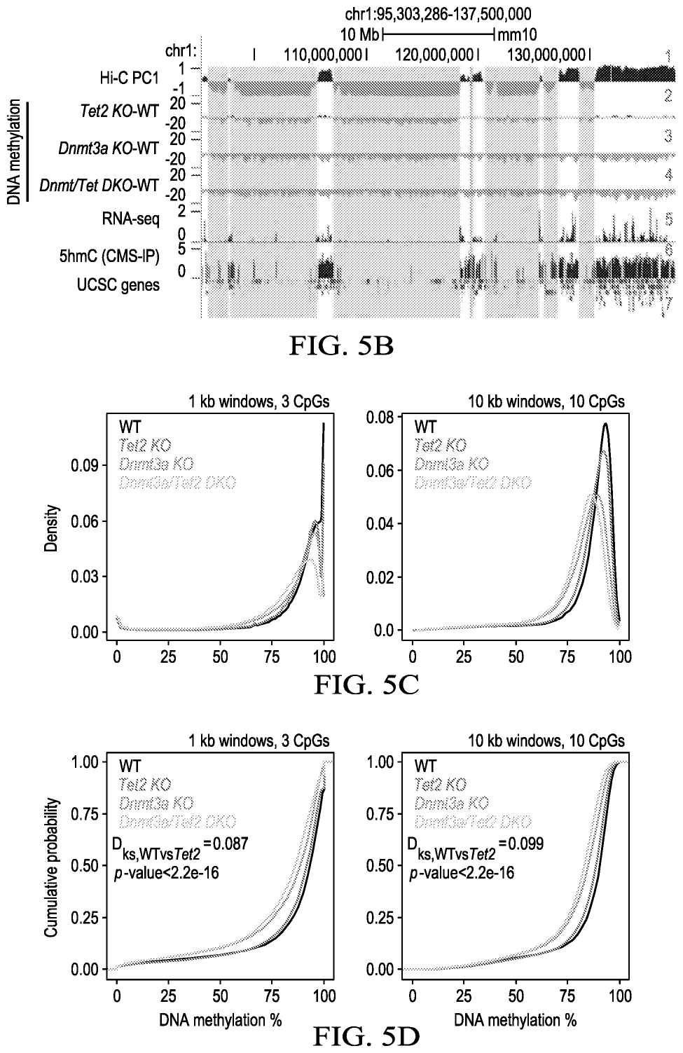

To determine if these findings were generally applicable to other cell types, the inventors integrated previously published Hi-C data from mouse pro-B cells (32) with WGBS data from WT or Tet2−/− Tet3fl/fl Mb1 Cre pro-B cells (26) (FIG. 9C-9E). Deficiency of Tet2 and Tet3 in Tet2−/− Tet3fl/fl Mb1 Cre mice results in defective immunoglobulin light chain rearrangement and a consequent block of B cell development at the pro-B to pre-B transition (26). Again, it was observed that concurrent increases and decreases of DNA methylation in pro-B cells genome-wide, with hypermethylation again occurring in the euchromatic compartment and hypomethylation in the heterochromatic compartment (FIG. 9C-9E). Very similar results were obtained for mouse T cells and HSPC (see below, FIGS. 3, 5).

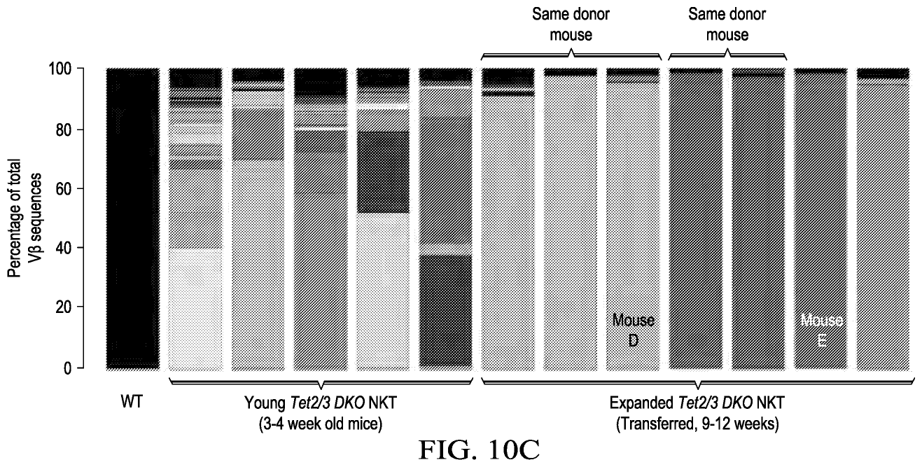

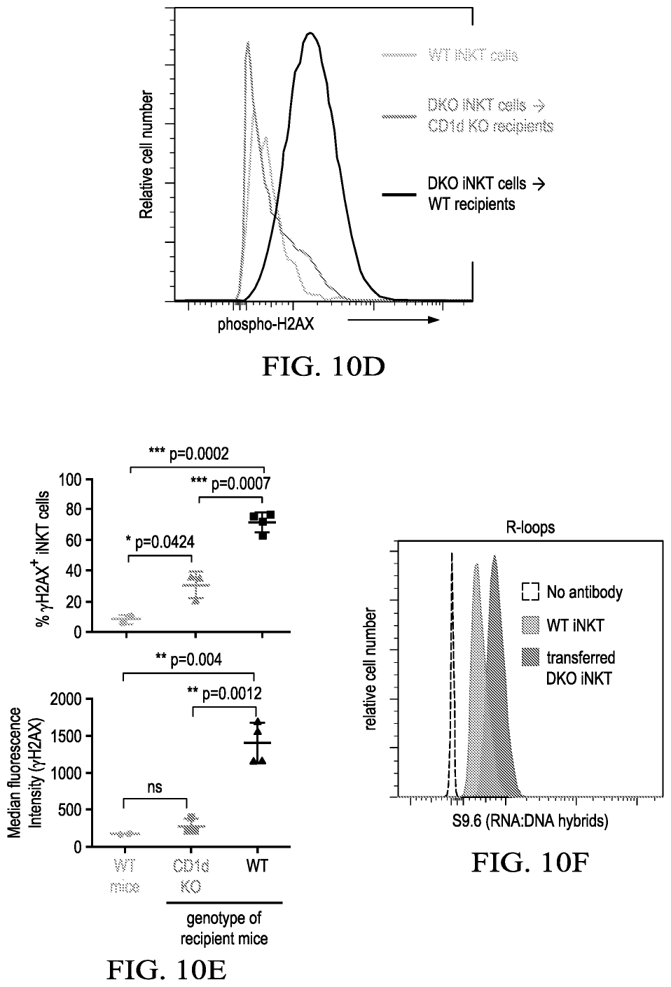

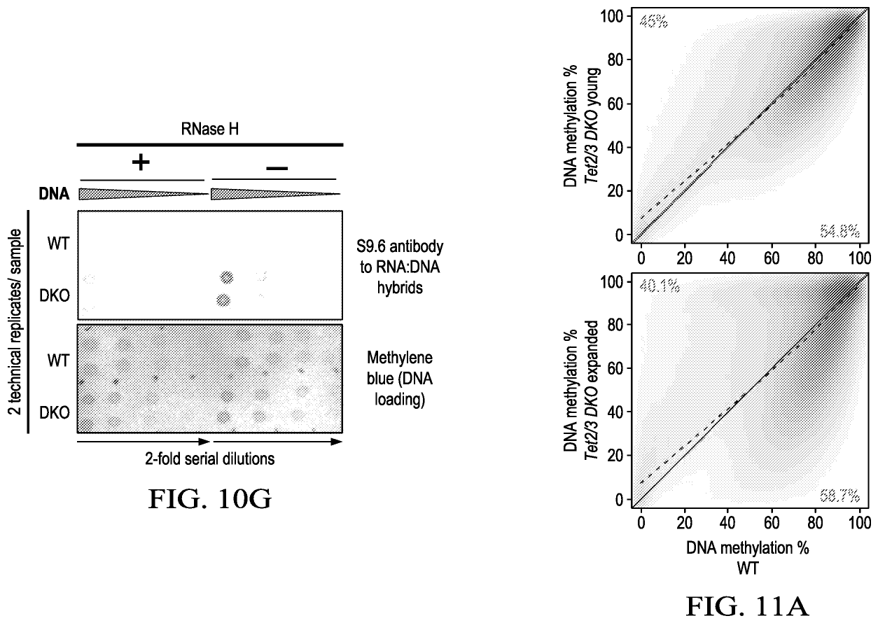

Antigen-driven expansion, increased clonality and DNA damage in TET-deficient T cell leukemia/lymphoma. To examine hypomethylation induced by TET loss-of-function in the context of oncogenic transformation, the inventors used a mouse model in which mice with profound TET loss-of-function (Tet2−/− Tet3fl/fl CD4Cre (Tet2/3 DKO) lacking Tet2 and Tet3 in T cells) rapidly developed an aggressive T cell leukemia/lymphoma with 100% penetrance (14). The disease involves a normally minor subset of T cells (iNKT cells, hereafter referred to simply as NKT cells), which recognize lipid antigens presented on a non-classical major histocompatibility complex (MHC) protein known as CD1d (33). Tet2/3 DKO mice showed>10-fold expansion of NKT cells in the thymus as early as 20 days after birth and in the spleen by 3-4 weeks, and succumbed to an NKT cell leukemia starting at 5 weeks (14). Transfer of purified NKT cells from young mice, even into fully immunocompetent recipients, resulted in transfer of the leukemia, but transfer to recipient mice lacking CD1d, the MHC protein that presents lipid antigen to NKT cells (33), resulted in minimal expansion (14) (FIG. 10A), indicating that the leukemia was driven by NKT cell expansion arising from presentation of lipid antigens by CD1d. The leukemia was transmissible indefinitely, and secondary transfers could be performed with as few as 50 cells (FIGS. 10A, 10B).

Sequencing of T cell receptor beta chain variable regions showed that Tet2/3 DKO NKT cells were oligoclonal even in young mice; after transfer to recipient mice, they displayed a remarkable increase in number (FIG. 10B) and clonality (FIG. 10C), due to due to strong selective expansion of cells with specific genomic characteristics (see below). Given that the expanded cells were effectively monoclonal and constituted>95% of lymphocytes in the peripheral lymphoid organs of recipient mice, the inventors were able to perform whole-genome sequencing (WGS), together with WGBS and Hi-C.

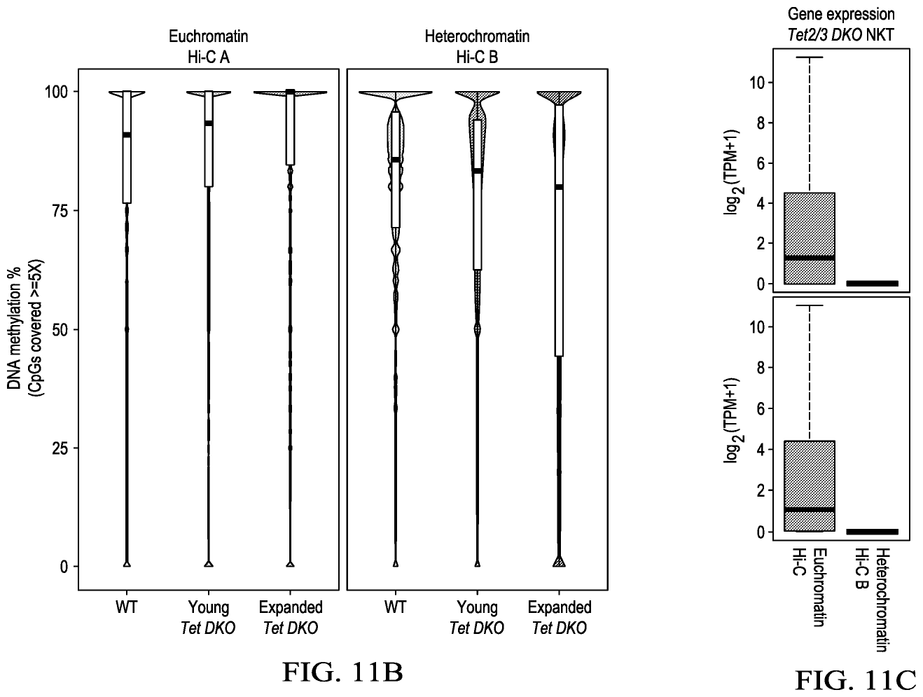

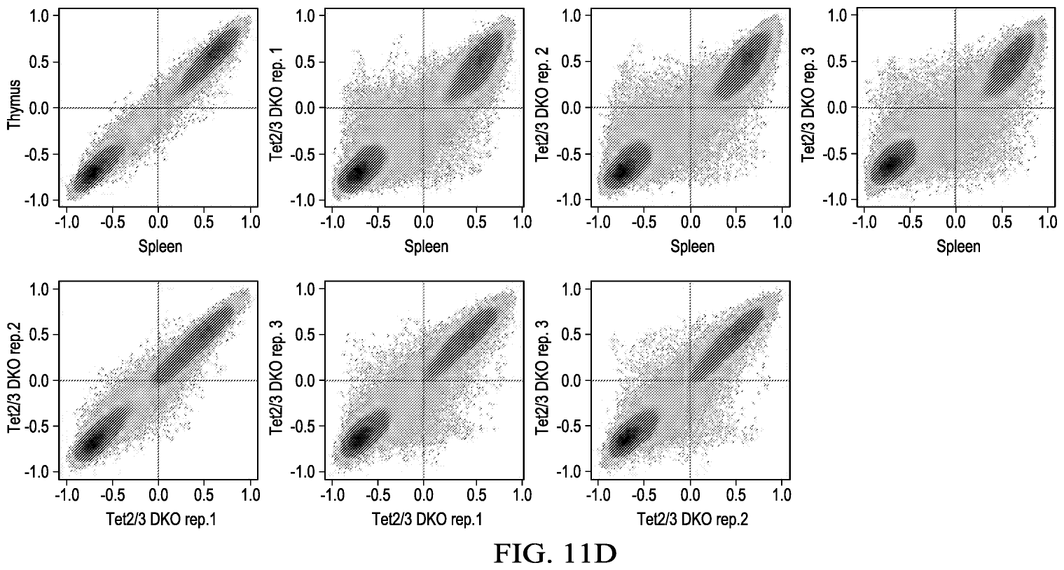

TET-deficient T cell lymphomas show euchromatic compartment hypermethylation and heterochromatic compartment hypomethylation. To define DNA modification patterns in the active and inactive genome compartments of TET-deficient NKT cell lymphomas, the inventors generated and analyzed WGBS and Hi-C data for WT, young, and transferred and expanded Tet2/3 DKO NKT cells. Like TET-deficient mESC and pro-B cells (FIGS. 1, 2, 8, 9), both young and expanded Tet2/3 DKO NKT cells showed increased methylation in the euchromatic compartment and decreased methylation in the heterochromatic compartment relative to WT NKT cells (FIG. 3A-3C; 11A, 11B). Moreover, as in the cell types considered above, the euchromatic compartment was gene-rich and contained the majority of expressed genes, accessible chromatin regions and 5hmC (FIGS. 3D, 3E; 11C; also see FIG. 3F, tracks 9-11). In genome browser views, there were only minor changes in euchromatic and heterochromatic compartments between WT thymus and spleen (FIG. 3F, tracks 1, 2; FIG. 11D, top left panel), but somewhat greater differences between WT and expanded Tet2/3 DKO splenic NKT cells (FIG. 3F, tracks 2, 3; FIG. 11D, bottom panels). Windows that were less accessible in Tet2/3 DKO NKT cells compared to WT were primarily in the euchromatic compartment; the few windows that were more accessible in Tet2/3 DKO cells compared to WT were present in the heterochromatic compartment (FIG. 3D). Finally, both young and transferred/expanded Tet2/3 DKO NKT cells showed extended regions of increased and decreased methylation compared to WT NKT cells, and these largely coincided with the euchromatic and heterochromatic compartments respectively (FIG. 3F, tracks 4-8).

Overall, the data on TET-deficient NKT cell lymphomas were completely concordant with those for the other TET-deficient cell types considered above. Regardless of cell type, TET deficiency was broadly associated with DNA hypermethylation in the euchromatic compartment, concurrently with DNA hypomethylation in the heterochromatic compartment. In the remainder of this disclosure, the inventors examine other genomic features of Tet2/3 DKO NKT cells reported to be associated with hypomethylation, including reactivation of repeat elements and increased mutational load.

Mutational signatures in TET-deficient T cell lymphomas. Hypomethylation has been previously associated with increased mutation rates (34) and genome instability (35, 36), and increased levels of DNA damage have been observed after TET deletion (9, 16). Expansion of Tet2/3 DKO NKT cells after transfer was accompanied by a striking increase in DNA double-strand breaks (DSBs): expanded Tet2/3 DKO NKT cells showed increased staining for γH2AX, compared to WT NKT cells (FIG. 10D, 10E). In contrast, Tet2/3 DKO NKT cells transferred to CD1d KO recipient mice, which undergo only minimal expansion (14) (FIG. 10A), displayed only a slight increase in DSBs compared to WT NKT cells (FIG. 10D, 10E). These results are consistent with the reports of increased DNA DSBs in Tet1-deficient B cells (9) and in acute myeloid leukemias resulting from inducible deletion of Tet2 and Tet1 (16).

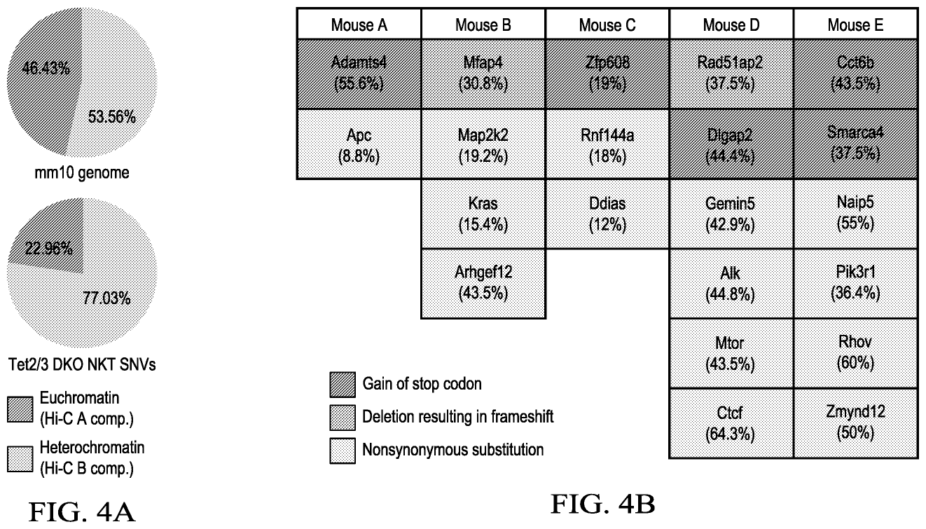

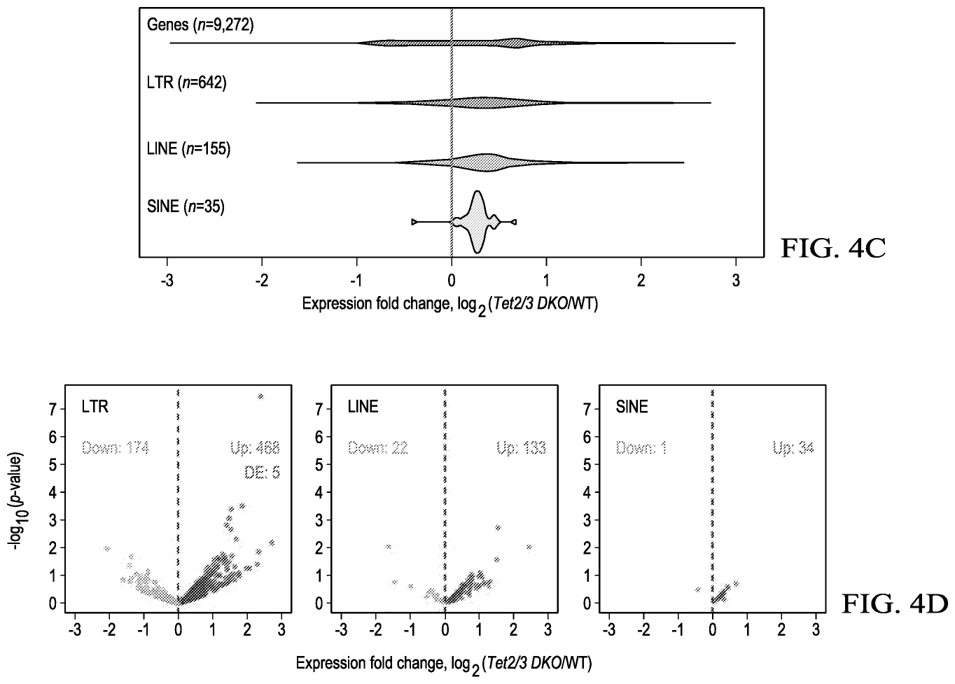

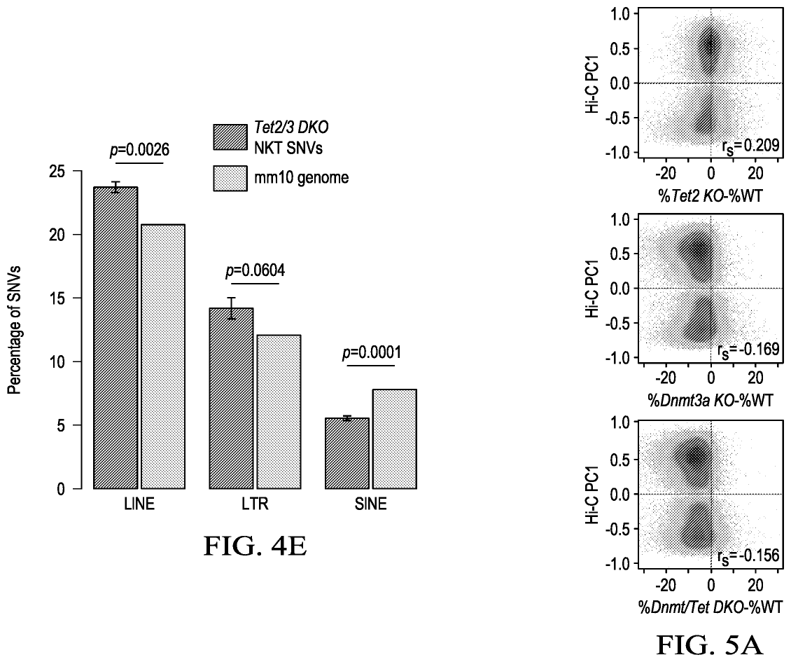

The inventors performed whole-genome sequencing (WGS) at >20× coverage. The inventors examined this WGS data to identify single-nucleotide variations (SNVs) in WT versus Tet2/3 DKO NKT cells. WGS on expanded NKT cells showed that most SNVs occurred in the largely heterochromatic compartment, which constitutes 54% of the genome but contains 77% of the SNVs (FIG. 4A). Furthermore, the SNVs identified in gene coding regions in the five different samples of transferred and expanded NKT cells from independent recipient mice (FIG. 4B) were not recurrently observed, providing that the selective advantage conferred by any given SNV is limited to individual mice. Thus most SNVs observed in Tet2/3 DKO NKT cells after transfer and expansion arise through a stochastic process occurring primarily in the heterochromatic compartment, as also observed for H3K9me3-marked heterochromatic genome regions in human cancers (24).

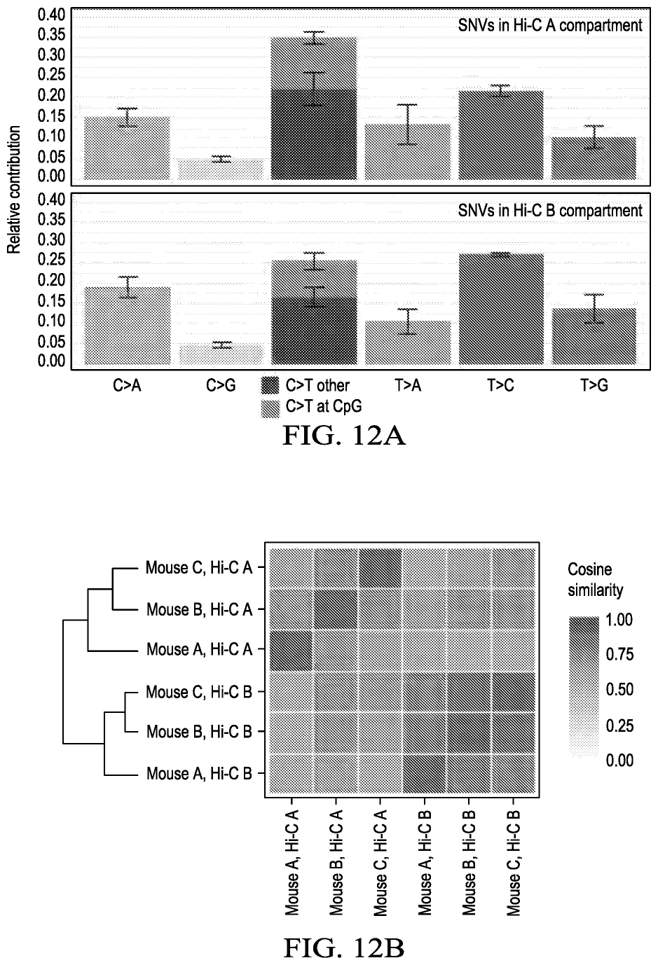

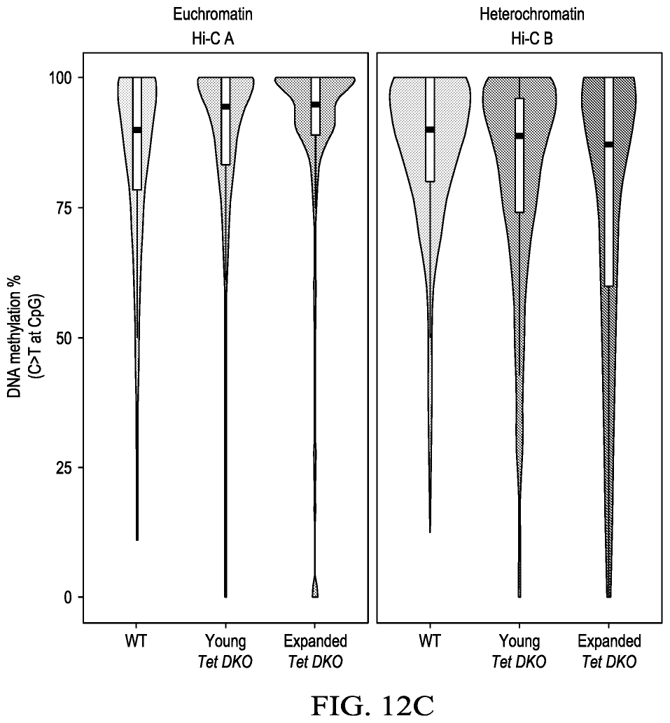

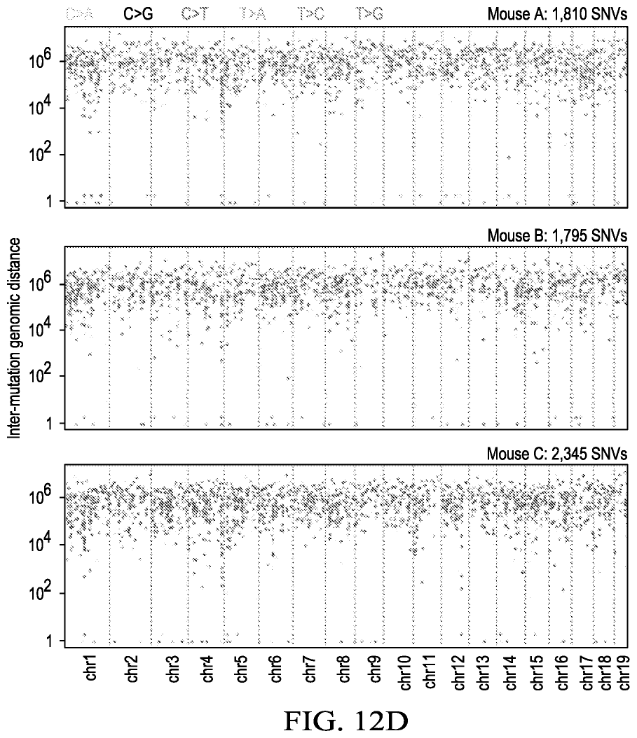

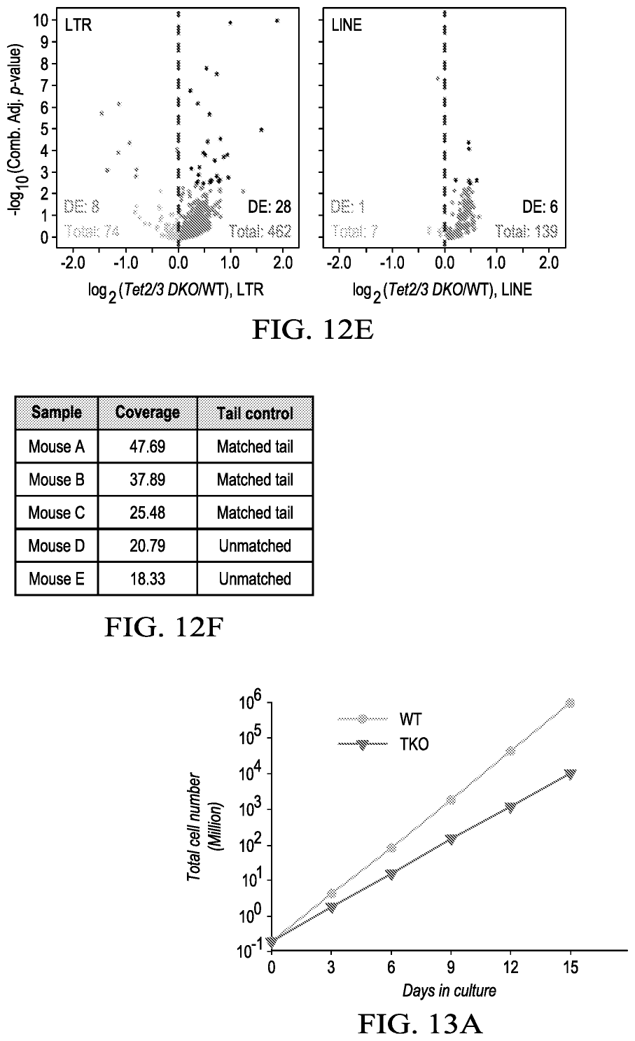

The mutational signature of the SNVs, based on nucleotide substitutions and sequence context at the 5′ and 3′ ends (37), clustered separately between the euchromatic and heterochromatic compartments (FIG. 12A, 12B). In both compartments, the signature was predominantly characterized by transitions (C>T, T>C, G>A and A>G). Even though SNVs in general were more prevalent in the heterochromatic compartment, SNVs at cytosines in the CpG context were more prevalent in the hypermethylated euchromatic compartment (14%) compared to the hypomethylated heterochromatic compartment (8.6%) (FIG. 12A, compare C>T red bars in top and bottom panels), as expected from the tendency of 5mC to undergo spontaneous deamination (37). Indeed, for CpGs for which DNA methylation data were available from WGBS analysis, the inventors observed that C>T mutations in the CpG context occurred at CpG sites that were largely methylated (FIG. 12C) Rainfall plots of intermutational distance against genomic location from three independent Tet2/3 DKO mice showed that mutations were often clustered in similar chromosomal locations but did not occur at the same nucleotides (FIG. 12D).

Reactivation of transposable elements in TET-deficient T cell lymphomas. DNA hypomethylation has been widely associated with reactivation of transposable elements (TEs) (38). In light of the hypomethylation in heterochromatin of Tet2/3 DKO NKT cells, the inventors analyzed the expression levels of distinct families of TEs in young Tet2/3 DKO NKT cells by RNA-seq (FIG. 4C-4D), keeping in mind that long interspersed elements (LINEs) are primarily located in heterochromatin while short interspersed elements (SINEs) are found in euchromatin (18). Indeed, using RNA-seq datasets from the inventor's previous experiments (14) as well as newly generated RNA-seq data from total ribodepleted RNA (FIG. 4C, D; FIG. 12E), the inventors found that for those TEs for which it was possible to detect transcripts reliably in at least one biological replicate, LTR and LINEs were more highly expressed in Tet2/3 DKO NKT cells with respect to WT, whereas SINEs remained largely unchanged. Furthermore, a substantial fraction of the identified mutations fell within TEs, such as LINEs and LTRs, but appeared underrepresented in SINEs with respect to the genome average (FIG. 4E). These data support the hypothesis that the reactivation of LINEs and LTRs results from TET-associated hypomethylation occurring in heterochromatin, whereas the euchromatic compartment undergoes TET-associated hypermethylation and therefore most SINEs remain silent.

Reactivation and spurious transcription of repeat elements has been associated with formation of R-loops and genome instability (39, 40), linked to DNA damage and DNA double-strand breaks (41). Indeed, the inventors found an increase of R-loops in expanded Tet2/3 DKO compared to WT NKT cells, as detected by flow cytometry and DNA dot blots using the S9.6 antibody against RNA:DNA hybrids (42) (FIG. 10F, 10G).

Paradoxical increase in heterochromatic DNA hypomethylation in HSPC from Dnmt3a-Tet2 DKO mice. The inventors used previously published Hi-C data on WT HSPC (45) and WGBS data for WT, Tet2 KO, Dnmt3a KO and Dnmt3a/Tet2 DKO HSPC (27, 46) to localize DNA methylation changes to the two genomic compartments defined by Hi-C (FIG. 5A; FIG. 5B, top track). Both Tet2 and Dnmt3a deficiency were characterized by widespread losses of DNA methylation in HSPC; moreover, HSPC from doubly Tet2/Dnmt3a-deficient mice showed greater hypomethylation than HSPC with either mutation alone (FIG. 5). The synergistic loss of DNA methylation was striking when 10 kb windows were considered (FIG. 5C, 5D, right panels), although the small fraction of fully methylated regions as well as completely or almost completely demethylated regions (e.g. CpG islands) were best observed in 1 kb windows (FIG. 5C, 5D, left panels). Specifically, Dnmt3a-deficient (Dnmt3a KO) HSPC displayed greater loss of methylation compared to Tet2-deficient (Tet2 KO) HSPC as expected, but HSPC doubly deficient in Tet2 and Dnmt3a (Dnmt3a/Tet2 DKO) showed even greater loss of methylation compared to HSPC singly deficient in either enzyme alone (FIG. 5C, 5D).

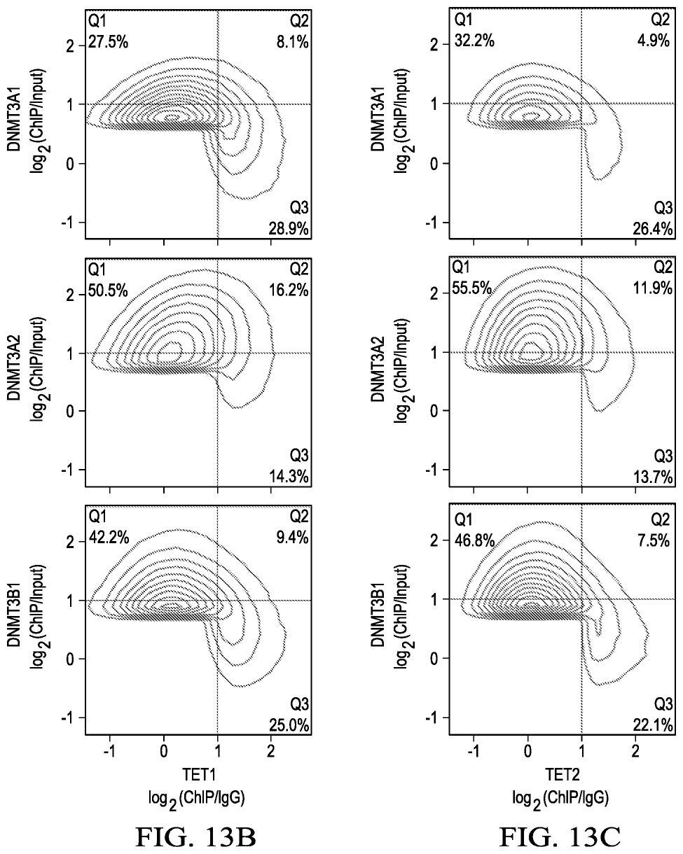

In genome browser views, extended domains of hypomethylation were observed in Tet2 KO HSC (FIG. 5B, track 2), as in all the other cell types considered above (FIGS. 1-3; 8, 9). These partially methylated domains mostly overlapped with the heterochromatic compartment, whereas regions in the euchromatic compartment showed a slight gain of methylation (FIG. 5A, top panel; FIG. 5B, track 2). In contrast, in both the Dnmt3a KO and Dnmt3a/Tet2 DKO HSPCs (FIG. 5A, middle and bottom panels; FIG. 5B, tracks 3, 4), the inventors observed widespread DNA hypomethylation in both the euchromatic and heterochromatic compartments, as expected from the loss of Dnmt3a activity. Thus, both Tet2 and Dnmt3a mutations result in widespread hypomethylation in heterochromatic regions of the genome. Decreased localization of Dnmt3a in the heterochromatin compartment of TET-deficient ESC. The inventors investigated the mechanisms underlying the loss of methylation in heterochromatin in TET-deficient cells. One mechanism stems from the observation that similar hypomethylated domains are observed in rapidly proliferating cells (22). This argument is plausible for the NKT cell lymphoma in which TET deficiency accounts for rapid proliferation, but cannot apply in the case of ESC. PMDs are not observed in WT ESC and induced pluripotent stem cells (iPSC) despite the high proliferation rates of these cells (47); moreover, Tet1/2/3 TKO mESC do not proliferate faster than their WT counterparts (FIG. S6B from (48) reproduced here in FIG. 13A for the reader's convenience), but clearly show decreased DNA methylation at late-replicating, lamina-associated domains (FIG. 2). Finally, senescent cells that have stopped proliferating also show partially-methylated domains, with loss of methylation occurring predominantly in the heterochromatic compartment (49).

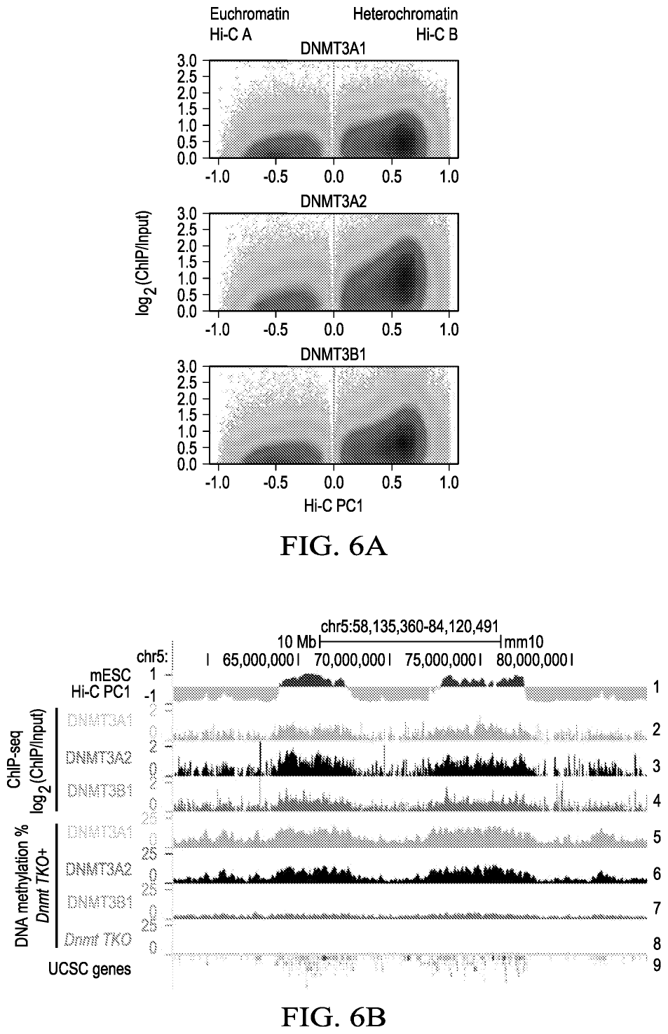

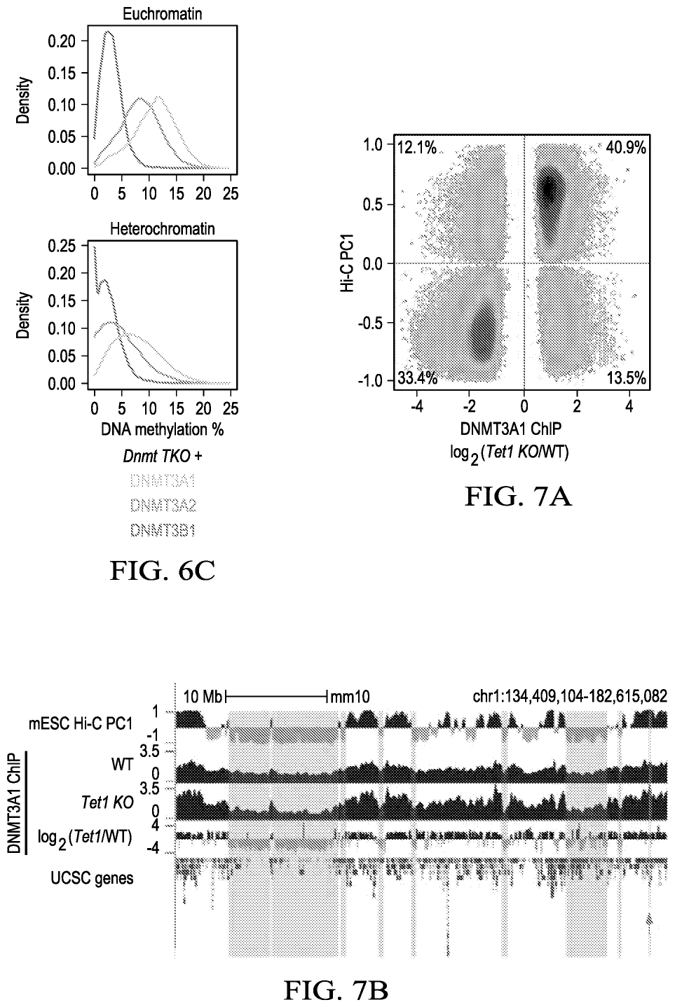

Since TET-deficient mESC showed heterochromatin hypomethylation without increased proliferation, the inventors investigated whether hypomethylation in mESC could be attributed to alterations of DNMT localization or function. To infer the contribution of each DNMT to methylation in the euchromatic and heterochromatic compartments, the inventors reanalyzed a dataset in which DNMT3B1 and the two splice variants DNMT3A1 and DNMT3A2 were reconstituted in mESC lacking all DNMTs (51, 52). Mapping of these three DNMT3 proteins in WT mESC showed that all three were primarily present in the euchromatic compartment but were also significantly represented in the heterochromatic compartment (FIG. 6A; FIG. 6B, tracks 1-4). WGBS performed on the DNMT-reconstituted cells showed that all three DNMT3 proteins contributed to methylation in both the euchromatic and heterochromatic compartments (FIG. 6B, tracks 5-8; FIG. 6C). Notably, the major contribution to DNA methylation in the heterochromatic compartment was from DNMT3A1 (FIG. 6C).

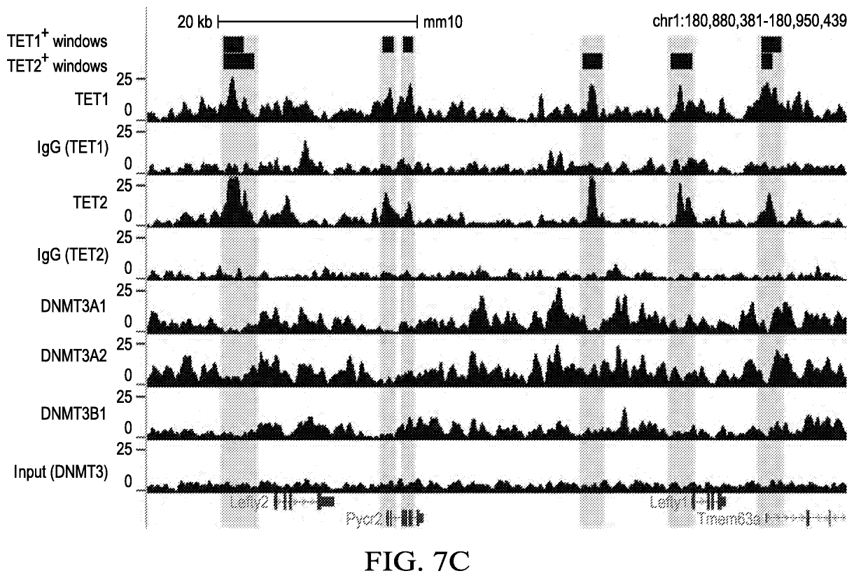

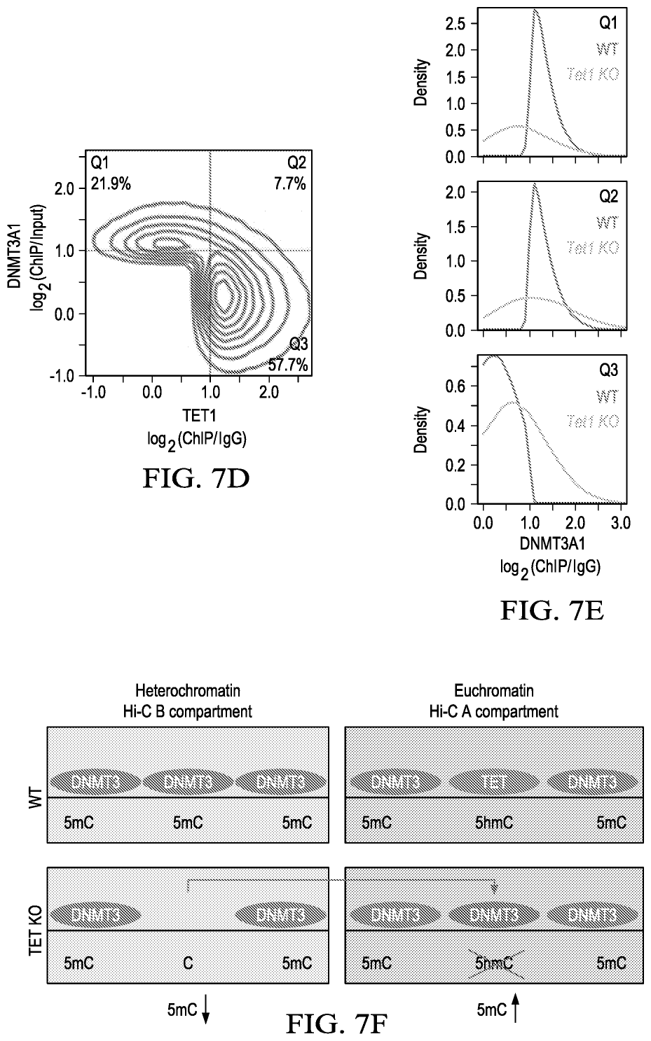

Heterochromatic hypomethylation in TET-deficient cells could reflect either altered distribution or function of DNMT3A1 (these two scenarios are not mutually exclusive). To examine alterations in DNMT3A1 localization, the inventors used a dataset from a study in which DNMT3A1 tagged with the biotin acceptor peptide for E. coli BirA was expressed in WT and Tet1-deficient mESC (53). The data show unambiguously that compared to WT mESC, DNMT3A1 was enriched in the euchromatic compartment and depleted from the heterochromatic compartment in Tet1-deficient mESC (FIG. 7A). A genome browser view illustrating the relocalization is shown in FIG. 7B; tracks 2, 3 show the normalized ChIP-seq coverage and track 4 shows the difference in DNMT3A1 binding in WT vs Tet1-deficient mESC. Together these data indicate that DNMT3A1 relocalizes from the heterochromatic to the euchromatic compartment in Tet1-deficient mESC.

The inventors used the same datasets described above (51-54) to determine how DNMT3A1 relocalized within the euchromatin compartment in Tet1-deficient mESC. A zoomed-in view of the Lefty1/2 locus within the euchromatic compartment revealed strong mutually exclusive localization of TET1/2 and DNMT3A (FIG. 7C). A contour plot illustrating this mutually exclusive localization is shown in FIG. 7D; Q1 contains the regions in the euchromatic Hi-C A compartment occupied by DNMT3A1 but not by TET1, whereas Q3 contains the regions occupied by TET1 but not by DNMT3A1. In Tet1-deficient mESC, this distribution is altered: the histograms for Q1 show that binding of DNMT3A1 to its exclusive sites in WT mESC is substantially decreased in Tet1 KO mESC, whereas the histograms for Q3 show that DNMT3A1 shows increased binding in Tet1 KO compared to WT mESC (FIG. 7E). This mutually exclusive localization seems to be a general feature of the relation between TET proteins and DNMT3s (FIG. 13B, 13C), as previously noted for TET1 and DNMT3A1 by (53). Together, the data indicate that loss of TET1 (almost exclusively from the euchromatic compartment) in mouse ES cells results in relocalization of DNMT3A1, on the one hand from the heterochromatic to the euchromatic compartment and on the other hand to regions within euchromatin that were previously occupied by TET1 (FIG. 7F). It is plausible that this relocalization contributes at least partly to the paradoxical loss of DNA methylation in the heterochromatic compartment that the inventors observe in TET-deficient cells, as well as to the hypermethylation observed in the euchromatic compartment of the same cells.

The inventors have shown an unexpected similarity between the DNA methylation patterns of diverse TET-deficient cell types and those of cancer genomes. Cancer genomes show local hypermethylation combined with widespread hypomethylation (23), and the inventors reproducibly observed both features in TET-deficient cells. As expected from the biochemical activities of TET enzymes in generating oxi-mC bases and their involvement in DNA demethylation (4), local DNA hypermethylation was consistently observed in the euchromatic Hi-C A compartment of TET-deficient cells; this compartment contains the vast majority of 5hmC, a stable modification that is most highly enriched in the gene bodies of the most highly expressed genes and at the most active enhancers (12, 14). The inventors also observed large domains of DNA hypomethylation in the heterochromatic Hi-C B compartment of diverse TET-deficient cell types, including ESC, NPC, HSPC, T cells and pro-B cells. These hypomethylated domains in TET-deficient cells provide that TET proteins are required, directly or indirectly, for optimal DNMT-mediated DNA methylation in heterochromatin.

To explore the biological consequences of TET loss-of-function in vivo, the inventors used a mouse model of profound TET deficiency in T cells. Mice with deletion of Tet2 and Tet1 genes in T cells showed early signal-dependent expansion and increased clonality, which rapidly progressed to an aggressive NKT cell lymphoma. The expanded Tet2/3 DKO NKT cells developed the same aberrations in DNA methylation—hypermethylation in the euchromatic compartment and hypomethylation in the heterochromatic compartment—that occur in cancer genomes and were noted herein for multiple TET-deficient cell types. The cells accumulated SNVs, largely in the hypomethylated heterochromatic compartment through an apparently stochastic process that differed in each individual mouse. The inventors also observed reactivation of transposable elements, particularly LTR and LINEs that are primarily located in heterochromatin; these repetitive elements were also more prone to mutations compared to the genome in general, recalling the genome instability produced by spurious transcription of repeat elements (39, 40). As described in more detail below, DNA hypomethylation in heterochromatin may at least partly explain the oncogenic transformation, genome instability and DNA damage observed in diverse mouse models of partial or profound TET deficiency (9, 16). The latency and penetrance of oncogenic transformation in these models depends on the extent of TET loss-of-function. Loss-of-function mutations in DNMT3A or TET2 are associated with clonal hematopoiesis in humans (55); similarly, TET deficiency in mouse models promotes the clonal expansion of TET-deficient cells. In both cases, full-blown oncogenesis requires the stochastic appearance of second hit mutations that vary from cell to cell but are subject to selection, driving clonal expansion and cancer evolution and explaining cancer heterogeneity.

The large hypomethylated domains observed in the heterochromatic compartment of TET-deficient cells are very reminiscent of the extended, partially methylated domains (PMDs) observed in cancer genomes. Based on their overlap with nuclear lamina-associated, late-replicating domains, cancer-associated PMDs occur in the heterochromatic compartment (20, 21); their presence has been attributed to ineffective DNMT1-mediated remethylation of late-replicating genomic regions in rapidly-proliferating cells (22). PMDs have also been documented in CD4⁺ T cells from a 103-year-old individual compared to those from a newborn human (22), providing that DNA methylation is also progressively lost in the heterochromatin of cells undergoing sustained long-term proliferation. While the presence of hypomethylated domains in heterochromatin of Tet2/3 DKO compared to WT NKT cells may indeed be a consequence of more rapid proliferation, especially since expanded Tet2/3 DKO NKT cells that have undergone many more cell divisions show more extensive hypomethylation than Tet2/3 DKO NKT cells from young mice (FIG. 3F), the PMDs observed in TET-deficient mESC cannot be explained by increased proliferation. Moreover, PMDs have also been observed in senescent IMR90 cells, which are no longer engaged in active proliferation (49). Thus, increased proliferation might contribute to, but is not the only mechanism underlying, the widespread losses of DNA methylation in heterochromatin of TET-deficient cells.

DNA hypomethylation has been associated with many biological consequences, including reactivation of transposable elements (38), sharply increased mutation rates (34), and genome instability with chromosome segregation defects and aneuploidies (35, 36). Mice with a hypomorphic mutation in Dnmt1 displayed genome-wide hypomethylation in all tissues and developed T cell lymphomas that occurred in 80% of mice and were characterized by recurrent aneuploidies (36). Reactivation of transposable elements is prevalent in cancer genomes, and is associated with the formation of RNA-DNA hybrids and R-loops (39, 40), which in turn have been linked to DNA damage and the appearance of DNA double-strand breaks (41). Each of these features was observed, together with heterochromatin hypomethylation, in expanded Tet2/3 DKO NKT cells. Thus in addition to their well-established role in promoting and maintaining DNA demethylation at promoters, gene bodies and enhancers, TET proteins participate in maintaining physiological levels of DNA methylation in heterochromatic compartments of the genome.

These findings may explain the unexpected synergy between TET2 and DNMT3A mutations in humans as well as mice. TET2 and DNMT3A are recurrently co-mutated in a diverse range of myeloid and lymphoid malignancies (43, 44). In a previous disclosure, the inventors found that the phenotypes of mice with dual Tet2 and Dnmt3a deficiency in HSPC were considerably more severe than those of mice with individual Tet2 or Dnmt3a deletions alone (27). Dnmt3a and Tet2 deficiency would both result in loss of oxi-mC at specific genomic regions, through a direct decrease in DNA methylation in the case of Dnmt3a deficiency and through loss of the 5hmC substrate in the case of Tet2 deficiency. Thus the stronger defects (e.g. in erythrocyte differentiation) in Tet2/Dnmt3a DKO mice compared to mice with Tet2 or Dnmt3a deficiency alone (27) arises from loss of “cooperation” between DNMT3A and TET2, leading to decreased 5hmC and increased 5mC at specific euchromatic locations (promoters, gene bodies, enhancers) in both humans and mice. Based on this data, however, the inventors have determined that pronounced DNA hypomethylation in the heterochromatic compartment of Tet2/Dnmt3a DKO HSPC (FIG. 5) could also be a major contributor to the observed synergy of oncogenic transformation resulting from loss-of-function mutations of both Dnmt3a and Tet2 (27).

This reanalysis of published data provides a mechanism for the synergistic actions of DNMT3A and TET proteins. TET1 and DNMT3A occupy mutually exclusive locations in the euchromatic compartment of mouse embryonic stem cells, and loss of TET proteins from euchromatin results in relocalization of DNMT3A1 to regions previously occupied by TET1 (see model in FIG. 7F). Broadly, this observation provides that the DNMT3 enzymes responsible for de novo methylation are recruited to euchromatin through a scaffold complex or other recruitment mechanism in common with TET enzymes, but for which the DNMTs have lower affinities than TETs under normal physiological conditions. Assuming that the DNMT3 enzymes are present at limiting concentrations, loss of TET proteins would cause them to relocalize away from heterochromatin and into euchromatic regions, resulting in the observed dual loss of DNA methylation in heterochromatin and increased DNA methylation in euchromatin. This observation is consistent with the notable finding that every animal genome that encodes a DNMT also harbors at least one functional TET/JBP protein (56).

This data provide that loss of DNA methylation in heterochromatin results in “heterochromatin dysfunction” (57). This phenomenon has many manifestations, including aneuploidies resulting from chromosome instability related to centromere dysfunction, as observed in ICF (immunodeficiency, centromere instability, facial abnormalities) patients with germline DNMT3B mutations (58), as well as reactivation of transposable elements and increased R-loops. These features are all observed in Tet2/3 DKO NKT cells, as well as in cells with hypomorphic mutations in DNMT1 (36). Based on these considerations, the inventors show that cancers related to TET loss-of-function are initiated at least partly through defects in the maintenance of heterochromatin function. By inference, the functional interactions between DNMT and TET proteins that are shown here are important for maintaining heterochromatin integrity.

In many hematopoietic and most solid cancers, TET loss-of-function is observed without coding region mutations in TET genes (5, 10). Early studies suggested that TET loss-of-function was secondary to TET promoter methylation, increased degradation of TET proteins, or aberrant microRNA expression (9-11). More recently, however, TET loss-of-function in solid cancers has been increasingly attributed to hypoxia (59), or to a variety of metabolic alterations that decrease ec-ketoglutarate levels or increase the levels of 2-hydroxyglutarate (2HG) (10, 11). Thus loss of DNA methylation in the heterochromatic compartment, and the consequent development of heterochromatin dysfunction could be the first steps in the development of many cancers characterized by TET loss-of-function. Moreover, mutations in proteins associated with the maintenance of heterochromatin integrity are frequent in cancer and many of them (e.g. NPM1) co-occur with TET2 mutations, leading to the postulate that heterochromatin dysfunction is not only a common feature of sporadic (non-hereditary) human cancers but also an initiating event in oncogenic transformation (57).

The methylation losses that were observed are fractional, only around 25% in this T cell lymphoma model, meaning that only a quarter of the total alleles in the transformed Tet2/3 DKO T cell population have lost the methyl mark at any given CpG. This heterogeneity of DNA methylation could affect the reactivation of transposable elements, the binding of methyl-sensitive proteins and transcription factors (60), thus contributing to the initiating events of transformation.

Thus, in particular embodiments, the elements of the present invention may decrease, reduce, inhibit, suppress or disrupt an immune or inflammatory response. In still further embodiments, the elements of the present invention may elicit, stimulate, induce, promote, increase or enhance an anti-cancer or anti-age-associated genome dysfunction, immune disorder, or autoimmune response, disorder or disease in a subject.

The elements of the present invention can be employed in various methods, uses and compositions. Such methods and uses include, for example, use, contact or administration of one or more elements of the present invention in vitro and in vivo. Such methods are applicable to providing treatment to a subject for cancer or an age-associated genome dysfunction, immune disorder, or autoimmune response, disorder or disease.

Methods and compositions of the invention include administration of the diagnostics, treatments, and agents disclosed herein, to a subject alone or in combination with any compound, agent, drug, treatment or other therapeutic regimen or protocol having a desired therapeutic, beneficial, additive, synergistic or complementary activity or effect.

The invention therefore provides treatments in combination with a second active, including but not limited to any compound, agent, drug, therapeutic regimen, treatment protocol, process, remedy or composition, such as a treatment protocol set forth herein or known in the art. The compound, agent, drug, therapeutic regimen, treatment protocol, process, remedy or composition can be administered or performed prior to, substantially contemporaneously with or following administration of elements disclosed herein to a subject. Specific non-limiting examples of combination embodiments therefore include the foregoing or other compound, agent, drug, therapeutic regimen, treatment protocol, process, remedy or composition.

In methods of the present invention, compositions are used for which there is a desired outcome, such as a therapeutic or prophylactic method that provides a benefit from treatment, vaccination or immunization, and can be administered in a sufficient or effective amount.

As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by elements disclosed herein alone, but optionally in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to a method of the invention, such as vaccination and therapeutic treatments.

The term “subject” refers includes but is not limited to a subject at risk of cancer or an age-associated genome dysfunction, immune disorder, or autoimmune response, disorder or disease, as well as a subject that has already developed cancer or an age-associated genome dysfunction, immune disorder, or autoimmune response, disorder or disease. Such subjects include mammalian animals (mammals), such as a non-human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of cancer or an age-associated genome dysfunction, immune disorder, or autoimmune response, disorder or disease known in the art.

Accordingly, subjects appropriate for treatment include those having or at risk of cancer or an age-associated genome dysfunction, immune disorder, or autoimmune response, disorder or disease, also referred to as subjects in need of treatment. Subjects in need of treatment therefore include subjects that have been previously had cancer or an age-associated genome dysfunction, immune disorder, or autoimmune response, disorder or disease or that have an ongoing cancer or an age-associated genome dysfunction, immune disorder, or autoimmune response, disorder or disease or have developed one or more adverse symptoms caused by or associated with cancer or an age-associated genome dysfunction, immune disorder, or autoimmune response, disorder or disease, regardless of the type, timing or degree of onset, progression, severity, frequency, duration of the symptoms.

Prophylactic uses and methods are therefore included. Target subjects for prophylaxis may be at increased risk (probability or susceptibility) of developing cancer or an age-associated genome dysfunction, immune disorder, or autoimmune response, disorder or disease. Such subjects are considered in need of treatment due to being at risk.

Subjects for prophylaxis need not be at increased risk but may be from the general population in which it is desired to protect a subject against cancer or an age-associated genome dysfunction, immune disorder, or autoimmune response, disorder or disease, for example. Such a subject that is desired to be protected against cancer or an age-associated genome dysfunction, immune disorder, or autoimmune response, disorder or disease can be administered treatment or agent described herein. In another non-limiting example, a subject that is not specifically at risk for cancer or an age-associated genome dysfunction, immune disorder, or autoimmune response, disorder or disease, but nevertheless desires protection against cancer or an age-associated genome dysfunction, immune disorder, or autoimmune response, disorder or disease, can be administered a composition or agent as described herein. Such subjects are also considered in need of treatment.

“Prophylaxis” and grammatical variations thereof mean a method in which contact, administration or in vivo delivery to a subject is prior to development of cancer or an age-associated genome dysfunction, immune disorder, or autoimmune response, disorder or disease. In certain situations it may not be known that a subject has developed cancer or an age-associated genome dysfunction, immune disorder, or autoimmune response, disorder or disease, but administration or in vivo delivery to a subject can be performed prior to manifestation of disease pathology or an associated adverse symptom, condition, complication, etc. caused by or associated with cancer or an age-associated genome dysfunction, immune disorder, or autoimmune response, disorder or disease. In such case, a composition or method of the present invention can eliminate, prevent, inhibit, suppress, limit, decrease or reduce the probability of or susceptibility to cancer or an age-associated genome dysfunction, immune disorder, or autoimmune response, disorder or disease, or an adverse symptom, condition or complication associated with or caused by cancer or an age-associated genome dysfunction, immune disorder, or autoimmune response, disorder or disease.

“Prophylaxis” can also refer to a method in which contact, administration or in vivo delivery to a subject is prior to a secondary or subsequent exposure or infection. In such a situation, a subject may have had a prior cancer or an age-associated genome dysfunction, immune disorder, or autoimmune response, disorder or disease or prior adverse symptom, condition or complication associated with or caused by cancer or an age-associated genome dysfunction, immune disorder, or autoimmune response, disorder or disease. Treatment by administration or in vivo delivery to such a subject, can be performed prior to a secondary or subsequent cancer or an age-associated genome dysfunction, immune disorder, or autoimmune response, disorder or disease. Such a method can eliminate, prevent, inhibit, suppress, limit, decrease or reduce the probability of or susceptibility towards a secondary or subsequent cancer or an age-associated genome dysfunction, immune disorder, or autoimmune response, disorder or disease, or an adverse symptom, condition or complication associated with or caused by or associated with a secondary or subsequent cancer or an age-associated genome dysfunction, immune disorder, or autoimmune response, disorder or disease.

Treatment of cancer or an age-associated genome dysfunction, immune disorder, or autoimmune response, disorder or disease can be at any time during the cancer or an age-associated genome dysfunction, immune disorder, or autoimmune response, disorder or disease. Certain embodiments of the present invention can be administered as a combination (e.g., with a second active), or separately concurrently or in sequence (sequentially) in accordance with the methods described herein as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by cancer or an age-associated genome dysfunction, immune disorder, or autoimmune response, disorder or disease, or an adverse symptom, condition or complication associated with or caused by cancer or an age-associated genome dysfunction, immune disorder, or autoimmune response, disorder or disease. Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

Doses can be based upon current existing protocols, empirically determined, using animal disease models or optionally in human clinical trials. Initial study doses can be based upon animal studies, e.g. a mouse, and the amount treatment or agent disclosed herein administered in an amount that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has previously had cancer or an age-associated genome dysfunction, immune disorder, or autoimmune response, disorder or disease, the onset, progression, severity, frequency, duration probability of or susceptibility of the symptom, condition, pathology or complication, the treatment protocol and compositions, the clinical endpoint desired, the occurrence of previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

The dose amount, number, frequency or duration may be proportionally increased or reduced, as indicated by the status of the subject. For example, whether the subject has previously had cancer or an age-associated genome dysfunction, immune disorder, or autoimmune response, disorder or disease, whether the subject is merely at risk of cancer or an age-associated genome dysfunction, immune disorder, or autoimmune response, disorder or disease, exposure or infection, whether the subject has been previously treated for cancer or an age-associated genome dysfunction, immune disorder, or autoimmune response, disorder or disease. The dose amount, number, frequency or duration may be proportionally increased or reduced, as indicated by any adverse side effects, complications or other risk factors of the treatment or therapy.

In the methods of the invention, the route, dose, number and frequency of administrations, treatments, and timing/intervals between treatment and disease development can be modified. In certain embodiments, a desirable treatment of the present invention will elicit robust, long-lasting immunity against cancer or an age-associated genome dysfunction, immune disorder, or autoimmune response, disorder or disease. Thus, in certain embodiments, invention methods, uses and compositions provide long-lasting immunity to cancer or an age-associated genome dysfunction, immune disorder, or autoimmune response, disorder or disease.

Certain embodiments of the present invention may be provided as pharmaceutical compositions.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

To increase a treatment as described herein comprising a vaccination, a composition of the present invention can be coupled to one or more proteins such as ovalbumin or keyhole limpet hemocyanin (KLH), thyroglobulin or a toxin such as tetanus or cholera toxin. Invention compositions can also be mixed with adjuvants. As demonstrated herein, in certain embodiments, the form of adjuvant with which the invention proteins or peptides are mixed may change whether the protein or peptide elicits an atherogenic or protective response in a subject.

Adjuvants include, for example: Oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria molina tree, see, e.g., “Saponin adjuvants”, Archiv. fur die gesamte Virusforschung, Vol. 44, Springer Verlag, Berlin, p243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

Salts may be added to a composition of the present invention. Non-limiting examples of salts include acetate, benzoate, besylate, bitartate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, megluminie, potassium, procaine, sodium, tromethyamine or zinc.

Chelating agents may be added to a composition of the present invention. Non-limiting examples of chelating agents include ethylenediamine, ethylene glycol tetraacetic acid, 1,2-bis(o-aminophenoxy)ethane-N,N,N′,N′-tetraacetic acid, Penicillamine, Deferasirox, Deferiprone, Deferoxamine, 2,3-Disulfanylpropan-1-ol, Dexrazoxane, Iron(II,III) hexacyanoferrate(II,III), (R)-5-(1,2-dithiolan-3-yl)pentanoic acid, 2,3-Dimercapto-1-propanesulfonic acid, Dimercaptosuccinic acid, or diethylene triamine pentaacetic acid.

Buffering agents may be added to a composition of the present invention. Non-limiting examples of buffering agents include phosphate, citrate, acetate, borate, TAPS, bicine, tris, tricine, TAPSO, HEPES, TES, MOPS, PIPES, cacodylate, SSC, MES or succinic acid.

Cosolvents may be added to a composition of the present invention. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel ad Soklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

An agent as described herein can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. Thus, even though the invention is generally not expressed herein in terms of what is not included, embodiments and aspects that expressly exclude compositions or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

Example 1: The inventors have found that many different pathways can result in heterochromatin dysfunction, heterochromatic DNA hypomethylation, or both. As noted in a recent review article: “Although highly enriched for repeated DNA sequences and containing few protein-coding genes, . . . heterochromatin plays critical roles in safeguarding the genome, including . . . telomere protection, suppression of transposon activity, and DNA repair . . . Heterochromatin dysfunction provokes genetic turmoil by inducing aberrant repeat repair, chromosome segregation errors, transposon activation, and replication stress and is strongly implicated in aging and tumorigenesis”. The inventors examined the interaction between heterochromatic DNA hypomethylation and hetero-chromatin dysfunction, defined the underlying connection, and established how these two processes contribute to clonal hematopoiesis, aging and oncogenesis.

Cancer genomes are characterized by two opposing patterns of aberrant DNA methylation: focal hypermethylation and widespread DNA hypomethylation. DNA hypermethylation at promoters and enhancers contributes to oncogenesis through transcriptional silencing of genes involved in DNA damage repair and tumor suppressors, and has been shown to reflect the impaired expression or activity of TET proteins.

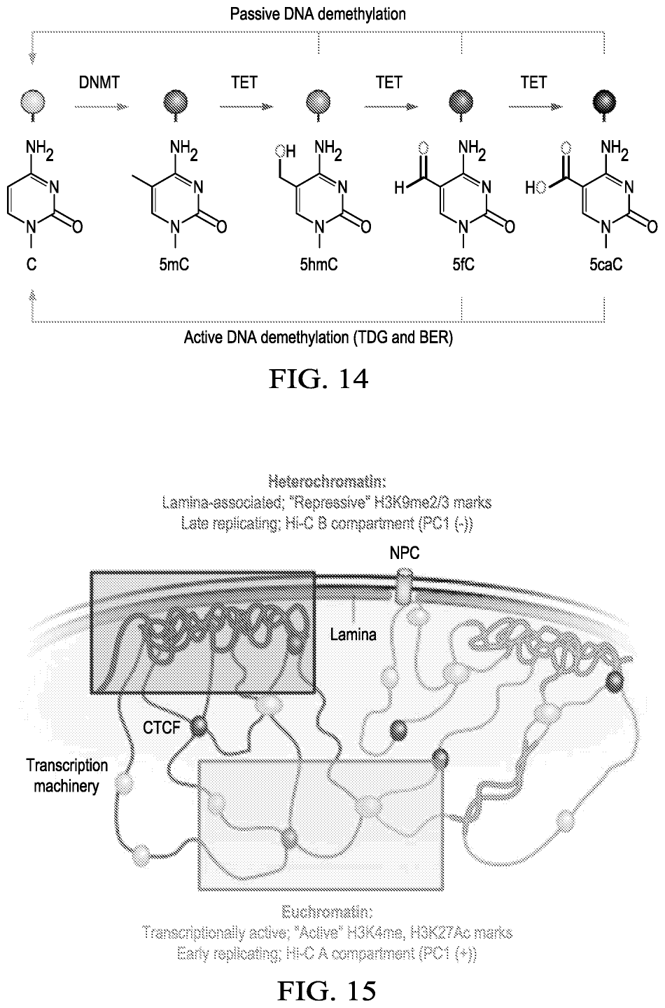

Biochemical activities of TET-family dioxygenases. DNA hypomethylation in heterochromatin and heterochromatin dysfunction are related. TET proteins mediate DNA demethylation by using Fe(II) and a-ketoglutarate (αKG, also known as 2-oxoglutarate, 2OG) to convert 5-methylcyto-sine (5mC) to 5-hydroxymethylcytosine (5hmC), 5-formylcytosine (5fC) and 5-carboxylcytosine (5caC) (FIG. 14)

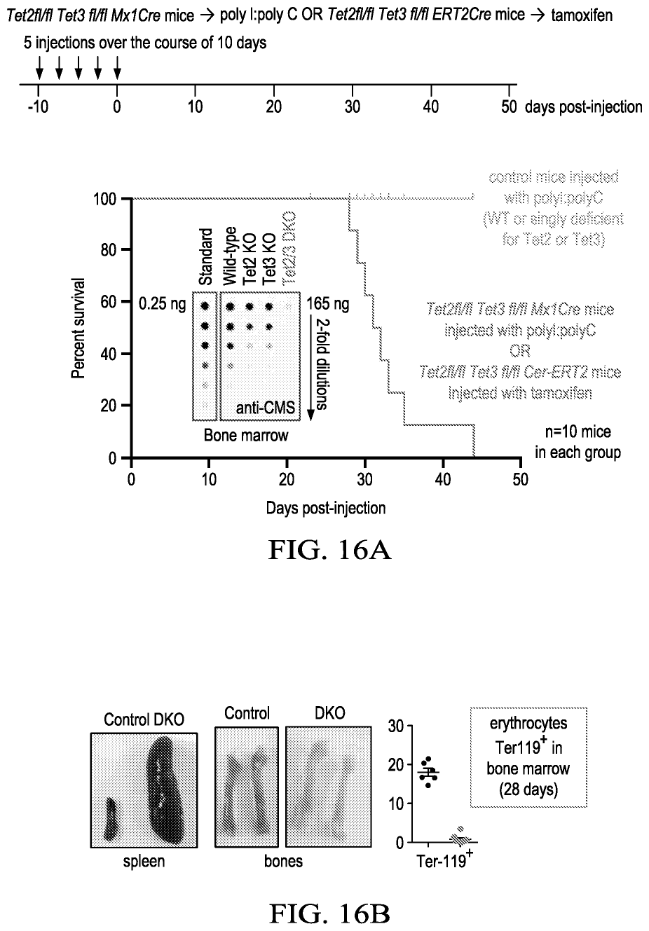

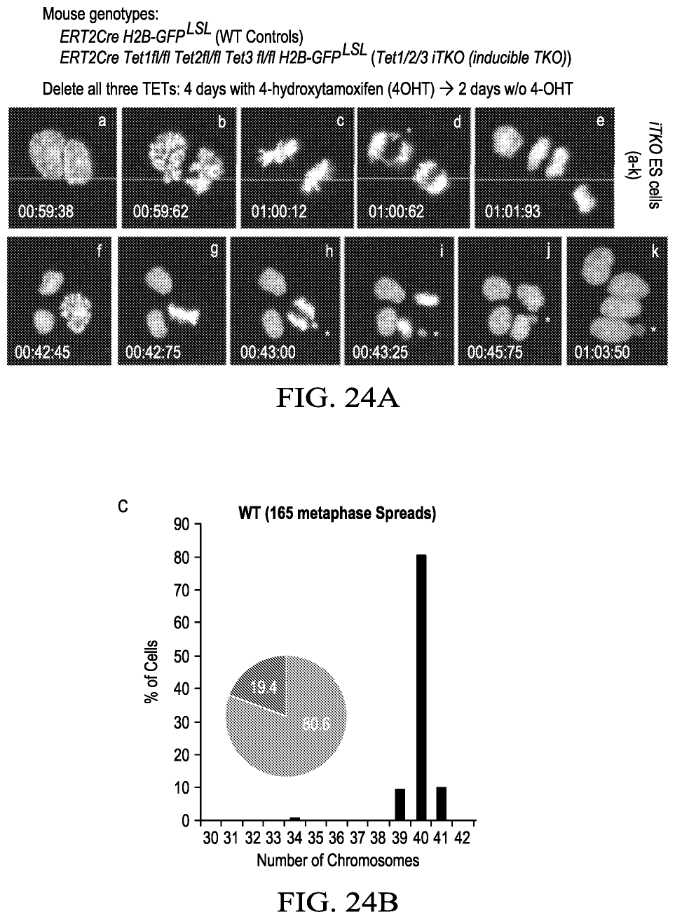

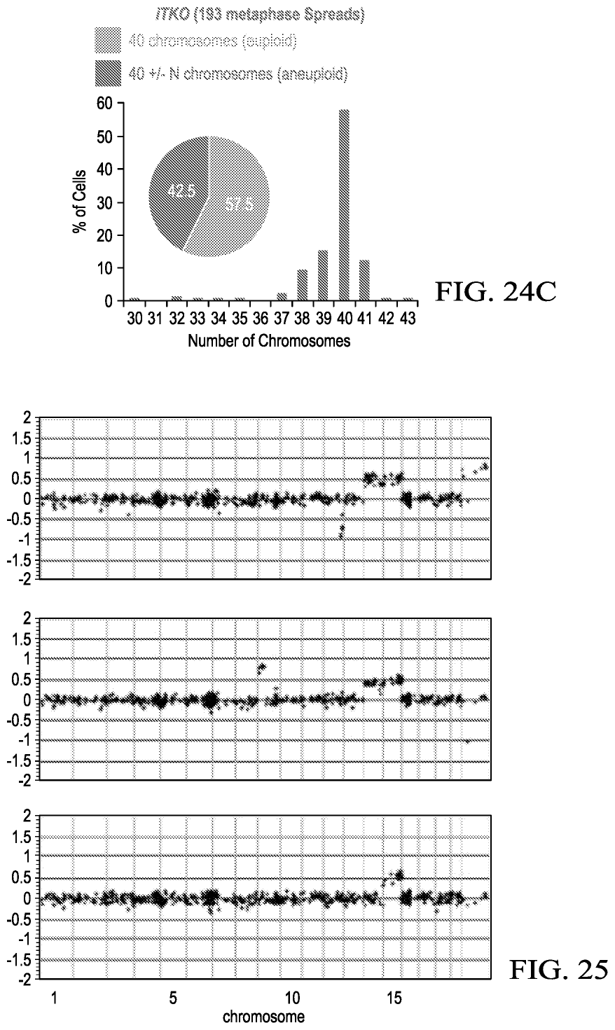

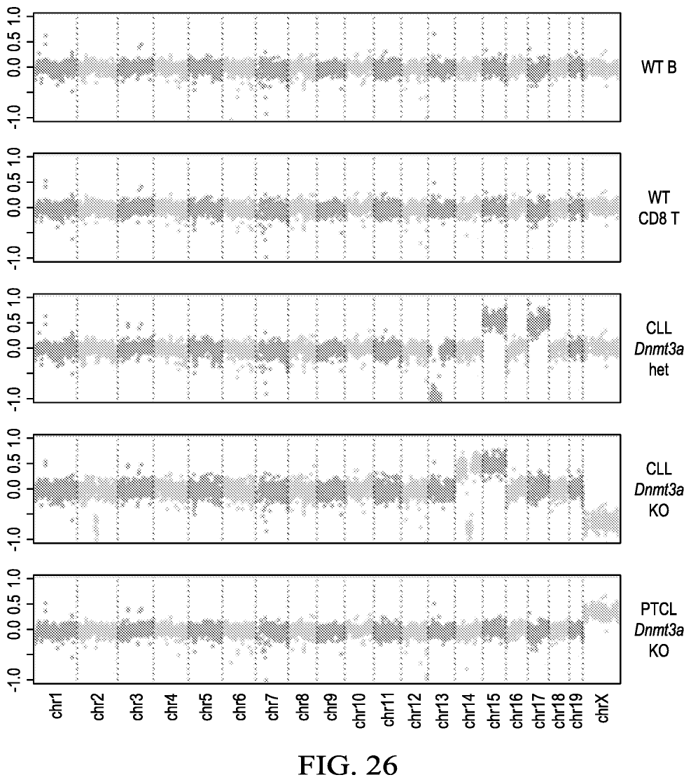

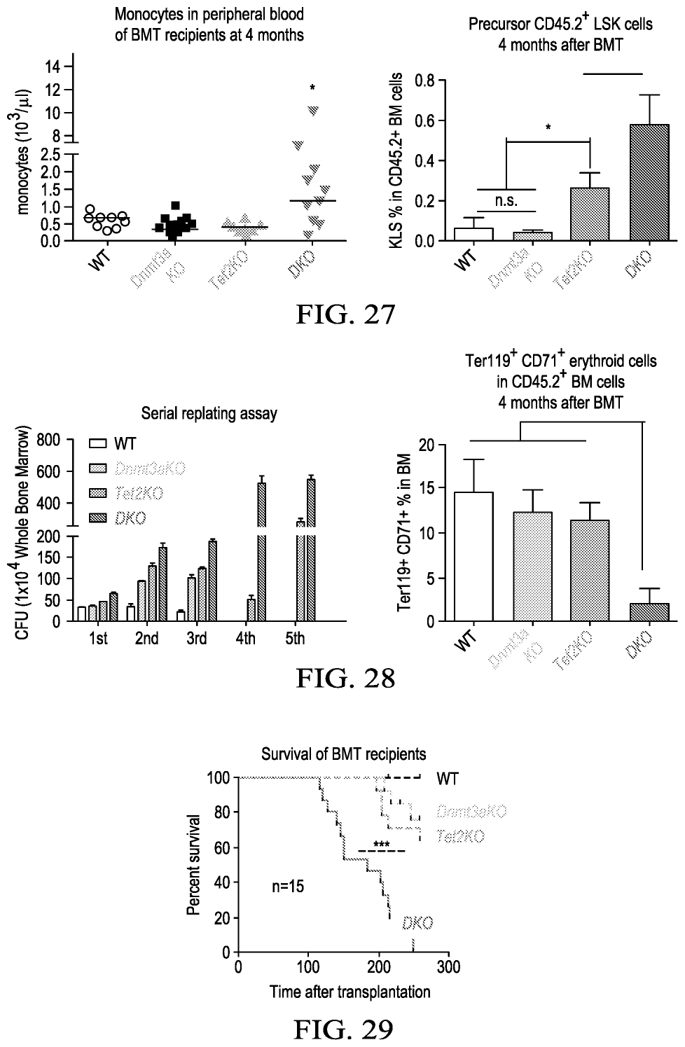

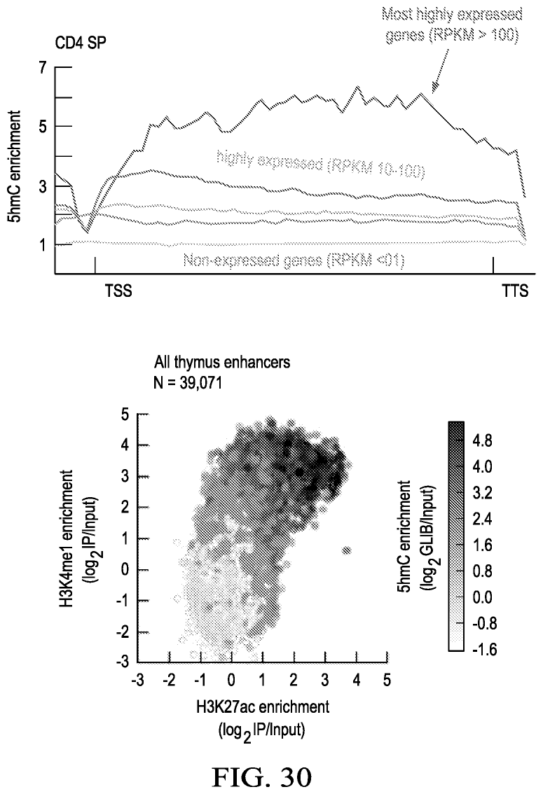

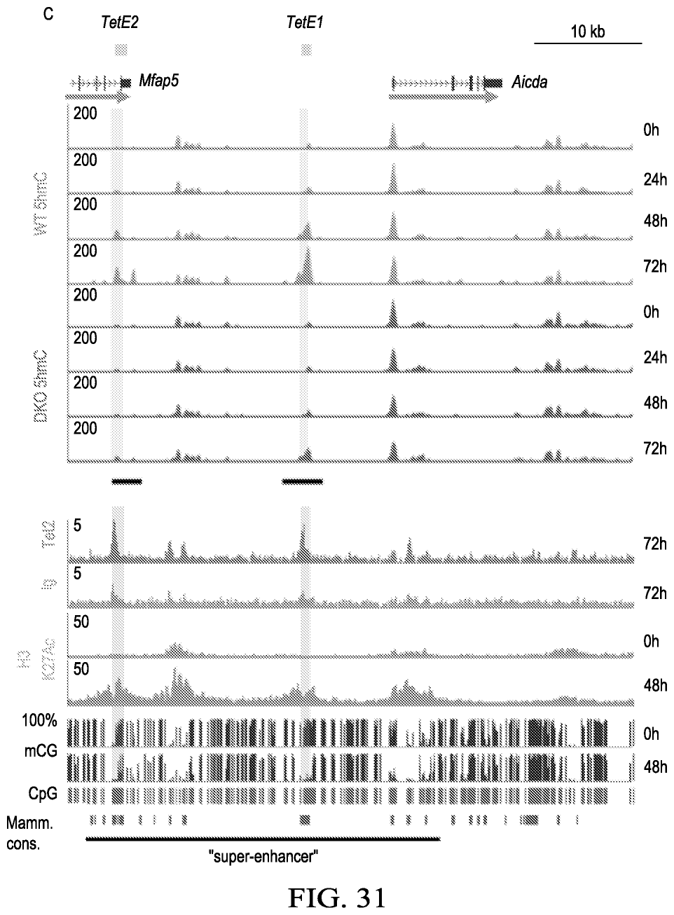

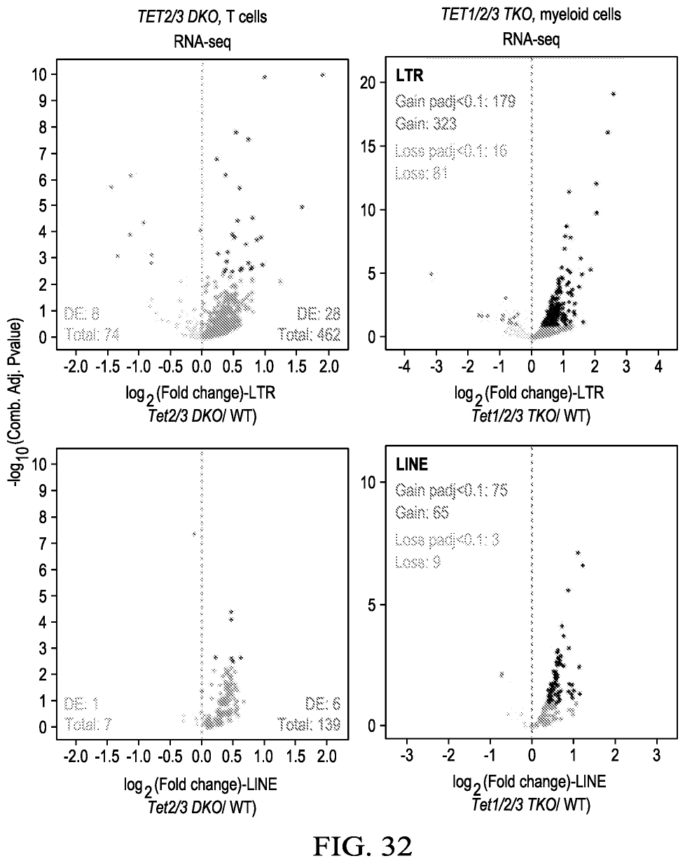

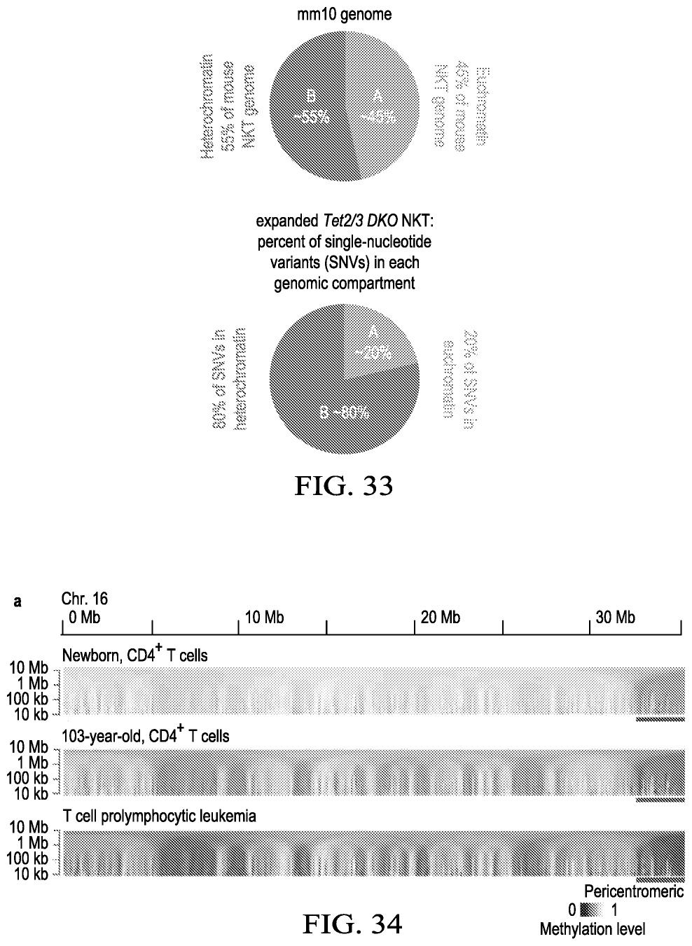

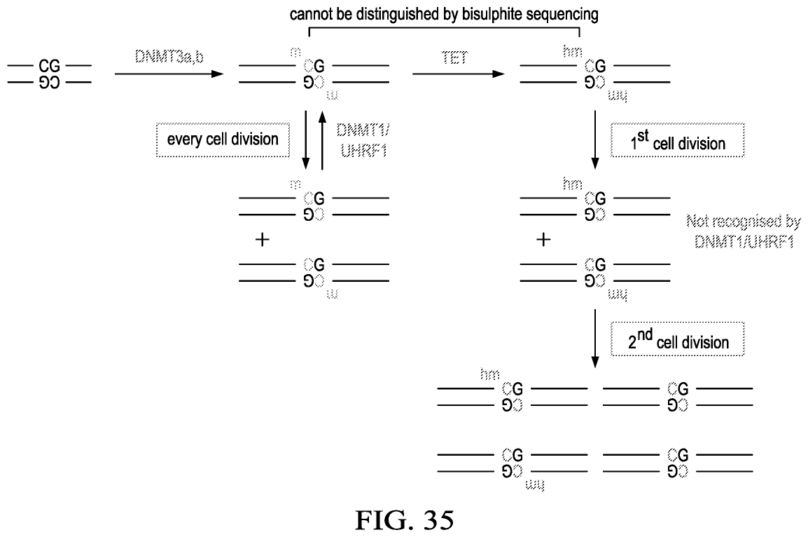

TET deficiency is strongly associated with lymphoid and myeloid malignancies. TET2 loss-of-function mutations are frequent in hematopoietic cancers—peripheral T cell lymphomas (PTCL), diffuse large B cell lymphomas (DLBCL), myelodysplastic syndromes (MDS), myeloproliferative neoplasms (MPN) and acute myeloid leukemia (AML). Similarly, the inventors have repeatedly shown that mice with TET deficiency—evoked by either developmental or inducible deletion of two or more TET genes in lymphoid or myeloid cells—rapidly develop aggressive, fully penetrant. The data are discussed in more detail below (FIGS. 16-21, 24, 25).

Heterochromatin and euchromatin can be distinguished by Hi-C. Data from unbiased, genome-wide chromosome conformation capture (Hi-C) experiments yield a matrix of interactions between all the different regions of the genome. Principal component analysis of this interaction matrix has been used to compartmentalize the genome into an A compartment (positive PC1 values) and a B compartment (negative PC1 values) that exhibit the hallmark characteristics of euchromatin and heterochromatin, respectively (55, 56). The euchromatic A compartment is rich in expressed genes in the cell type under consideration, whereas the heterochromatic B compartment is gene-poor and bears epigenetically “repressive” chromatin marks, including H3K9me2/3 (55, 56). Moreover, the Hi-C B compartment overlaps with lamina-associated domains and corresponds to late-replicating regions of the genome, while the Hi-C A compartment corresponds to early replicating genomic regions and is not lamina-associated (56, 57). Notably, the extended partially methylated domains (PMDs) observed in cancer genomes overlap with the Hi-C B compartment—late-replicating, nuclear lamina-associated domains (3, 58) (FIG. 15). Here, the Hi-C A and B compartments are referred to as euchromatic and heterochromatic compartments, respectively.

TET deficiency results in the expected increase of DNA methylation in euchromatin, but an unexpected decrease in DNA methylation in heterochromatin. Because TET enzymes biochemically mediate the loss of DNA methylation (FIG. 14), it was generally assumed that TET deficiency would result in increased DNA methylation, and that this in turn would result in onco-genesis by promoting the silencing of tumor suppressor genes. Indeed, increased DNA methylation is observed in the genomes of TET-deficient cells, and the inventors recently showed that this hypermethylation is confined to the euchromatic Hi-C B compartment. 5hmC is largely present at euchromatic regions—active enhancers marked by H3K4me1 and H3K27Ac, and gene bodies of the most highly expressed genes, to which TET proteins are recruited by transcription factors and RNA polymerase II. However, when the DNA methylation status of TET-deficient cell lines, and tumors that either arose naturally in humans or developed in mouse models of TET deficiency were analyzed, it was observed that TET loss-of-function also resulted in widespread losses of DNA methylation in randomly-chosen regions of the genome.

These considerations led the inventors to re-analyze and integrate whole-genome bisulfite sequencing (WGBS) and Hi-C (genome-wide chromosome conformation capture) data from many different TET-deficient cell types. The inventors found that the DNA methylation patterns of TET-deficient genomes closely resembled those of both aged and cancer genomes, with focal hypermethylation in euchromatin and widespread DNA hypomethylation in heterochromatin. The inventors partitioned the genome into heterochromatic and euchromatic compartments using Hi-C data, and examined DNA methylation status at megabase-scale resolution. The inventors used WGBS and Hi-C data. Data were derived from a variety of TET-deficient cells: mouse pro-B cells, expanded mouse myeloid cells, and expanded “NKT” cells with dual Tet2/Tet3 deficiency. Data from other labs were from mouse embryonic stem cells (mESC) with Tet1, Tet2 or triple Tet1/2/3 deficiency, neural precursor cells (NPC) differentiated from Tet2-deficient mouse ESC, and mouse hematopoietic stem/precursor cells (HSPC)). In every case, the inventors observed increased DNA methylation in the euchromatic Hi-C A compartment (positive PCI values), occurring concomitantly with loss of DNA methylation in the heterochromatic Hi-C B compartment. Selected data are described in FIGS. 16-21, 24, 25.

Association of DNMT deficiency and global loss of DNA methylation with features of heterochromatin dysfunction. Mice with a hypomorphic mutation in the maintenance DNA methyltransferase Dnmt1 and consequent genome-wide losses of DNA methylation developed T cell lymphomas with high penetrance, leading the inventors to conclude that “genome-wide hypomethylation plays a causal role in cancer”. T cell lymphomas developing in Dnmt1-hypomorphic mice showed frequent aneuploidies and copy number alterations (CNAs), as did the chronic lymphoid leukemias (CLL) and peripheral T cell leukemias (PTCL) developing in mice with conditional deletion of the de novo DNA methyltransferase Dnmt3a in mouse HSPC. Furthermore, humans with ICF syndrome (Immunodeficiency, Centromeric instability, and Facial anomalies), a fatal genetic disease caused in part by germline mutations in DNMT3B, show dramatic loss of DNA methylation at satellite repeats and recurrent aneuploidies of chromosomes 1, 9 and 16 (see below). This last feature may be cell type-specific, since lymphocytes of ICF patients show nuclear abnormalities whereas their fibroblasts do not.

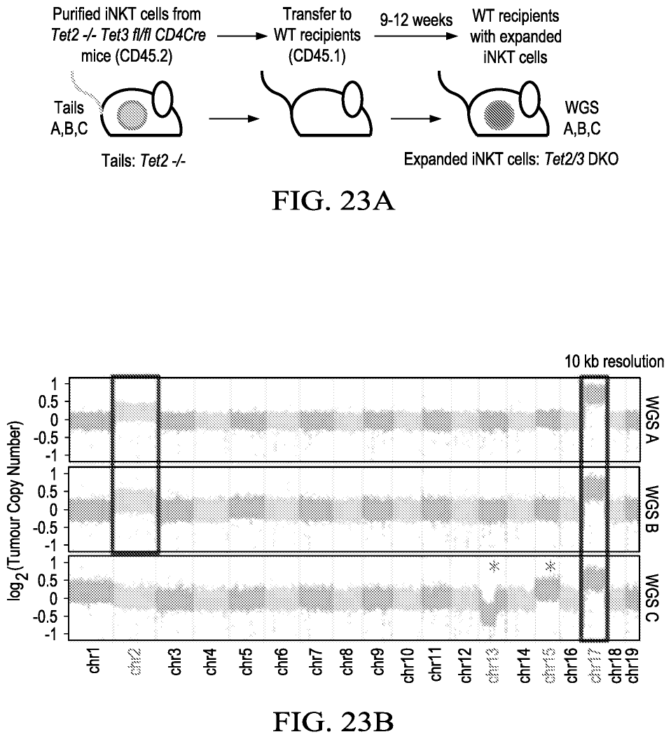

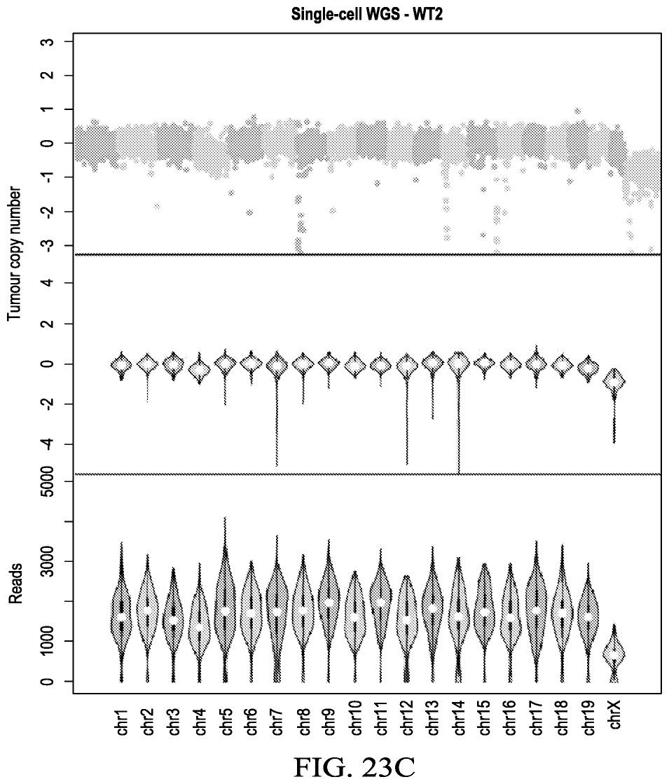

Notably, a comparable propensity to aneuploidies and CNAs was observed in Tet2/3-deficient expanded NKT cells (see below, FIG. 23). Overall, the inventors conclude that despite the opposing biochemical functions of TETs and DNMTs (FIG. 14), both TET-deficient and Dnmtl-hypomorphic T cells show heterochromatic DNA hypomethylation, accompanied by features of hetero-chromatin dysfunction (aneuploidies, reactivation of repeat elements, DNA damage etc.).

Synergistic biological effects of Dnmt3a and Tet2 mutations in mice. DNMT3A and TET2 mutations are frequently observed, both individually and together, in diverse myeloid and lymphoid malignancies including MDS, AML and PTCL; and Dnmt3a- and Tet2-deficient mice have unexpectedly similar disease phenotypes. Based on the biochemical activities of Tet2 and Dnmt3a, widespread losses of DNA methylation in Dnmt3a-deficient mice were expected, but widespread gains of DNA methylation in Tet2-deficient mice, concomitantly with loss of oxidized methylcytosines (oxi-mC) were observed in both cases. Mice with dual Tet2 and Dnmt3a deficiency in HSPC displayed more severe phenotypes than mice with individual Tet2 or Dnmt3a deletions alone. The changes in oxi-mC distribution were complex; but both Tet2 and Dnmt3a deficiencies were characterized by widespread losses of DNA methylation in HSP. HSPC from doubly Tet2/Dnmt3a-deficient mice showed greater loss of methylation than HSPC with either mutation alone; and the loss of methylation in Tet2-deficient HSPC was primarily in the heterochromatic compartment.

Given these findings, the inventors have shown that heterochromatic DNA hypomethylation in cancers of TET-deficient mice are responsible for the heterochromatin dysfunction and high propensity to oncogenic transformation that were observed in these cells. Notably, progressive DNA hypomethylation is also consistently observed in aged and senescent cells. Additionally, mutations in diverse other chromatin- and heterochromatin-associated proteins also appear to affect heterochromatin integrity, and many of these were previously shown to be associated with aging and cancer. The inventors found that heterochromatic DNA hypomethylation and heterochromatin dysfunction have a critical role in both aging and oncogenesis.

Example 2: TET proteins are recruited by transcription factors to promote gene expression in euchromatin. Using mouse model systems in which Cre recombinase was expressed at a specific phase of B, T, or regulatory T (Treg) cell development—Mb1Cre, CD4Cre and Foxp3-Cre respectively. In Tet2fl/fl Tet3 fl/fl Mb1Cre mice, the inventors traced a severe block in the pro-B to pre-B cell transition to a pronounced defect in immunoglobulin k (Igk) light chain rearrangement: because Tet2 and Tet3 were not available, they could not be recruited by the PU.1 and E2A transcription factors to two key enhancers in the Igk locus. Similarly when the inventors inducibly deleted Tet2 and Tet3 genes during stimulation of naïve B cells with lipopolysaccharide (LPS) and the cytokine Interleukin 4 (IL-4), a major decrease in class switch recombination was observed, which was traced to decreased expression of the activation-induced cytidine deaminase AID. Again, this reflected the fact that Tet2 and Tet3 were unavailable, and so could not be recruited by the transcription factor BATF to two TET-regulated enhancers in the Aicda (encoding AID) locus Similar data showing that transcription factors recruit TET proteins have been reported in other systems: for instance, WT1, KLF4, CEBPa etc recruit TET2 to enhancers and gene regulatory elements during myeloid differentiation.

Example 3: Notable association of TET loss-of-function with cancer. The most striking phenomenon observed in mice with profound TET deficiency, evoked by either developmental or inducible deletion of two or more TET genes, was the rapid development of aggressive, fully penetrant cancers. For instance, (a) even in the face of a profound block in pro-B to pre-B cell differentiation in Tet2fl/fl Tet3 fl/fl Mb1Cre mice, 100% of mice succumbed to an aggressive immature B cell lymphoma by 5 months of age; (b) mice with Tet2 and Tet3 deficiency induced in mature B cells with CD19Cre showed massive B cell expansion that led to death within 20 weeks; and (c) mice with Tet2 and Tet3 deficiency induced in T cells using CD4Cre displayed T cell malignancy with dramatic antigen-driven expansion and oncogenic transformation of “NKT” cells, a normally minor subpopulation of T cells that develop in the thymus and are self-reactive, but undergo positive selection rather than deletion in the thymus. The uncontrolled expansion of NKT cells in Tet2fl/fl Tet3 fl/fl CD4Cre mice is antigen- and signal-dependent, and results in the development of an increasingly clonal NKT cell malignancy in all mice by 5 weeks of age.

The inventors have shown that TET deficiency was causal for the malignancy by acutely inducing TET deletion in adult mice. In early experiments, the inventors acutely induced Tet2 and Tet3 deficiency by treating adult Tet2fl/fl Tet3 fl/fl Mx1Cre mice with polyI:polyC, and Tet2fl/fl Tet3 fl/fl ERT2-Cre mice with tamoxifen. More recently, the inventors induced almost complete TET deficiency by treating Tet1 fl/fl Tet2fl/fl Tet3 fl/fl ERT2-Cre mice with tamoxifen. The outcomes were very similar the mice showed strong myeloid skewing and developed a myeloid malignancy resembling AML by ˜4 weeks (FIG. 16). The data prove that profound TET deficiency is causal for rapid oncogenesis.

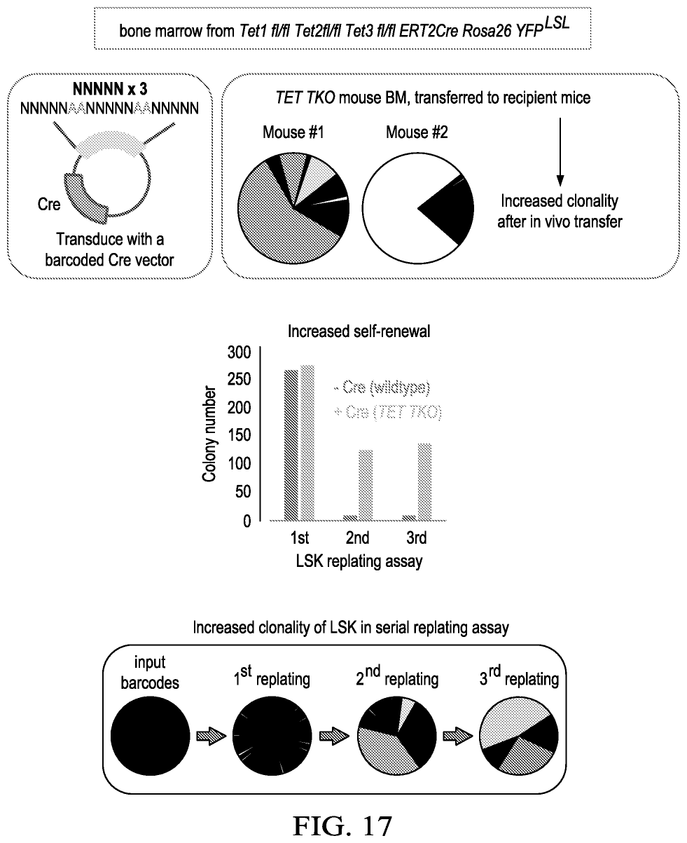

Example 4: The inventors performed bone marrow chimera experiments in which LSK (lineage-negative, Scal⁺, cKit⁺) cells, which are enriched for HSPC, were isolated from bone marrow of Tet1 fl/fl Tet2fl/fl Tet3 fl/fl ERT2-Cre mice, which also carried a Rosa26 YFP^{LSL} allele that is expressed upon Cre activation. The cells were transduced with a barcoded lentiviral vector encoding Cre, and transferred into irradiated recipient mice. All recipient mice developed an acute myeloid malignancy to which they succumbed by 70-75 days (2.5 months); the expanded cells were 100% YFP-positive, had deleted all three TET genes, and showed increased clonality (FIG. 17). Thus, myeloid expansion and oncogenic transformation depend on TET deficiency.

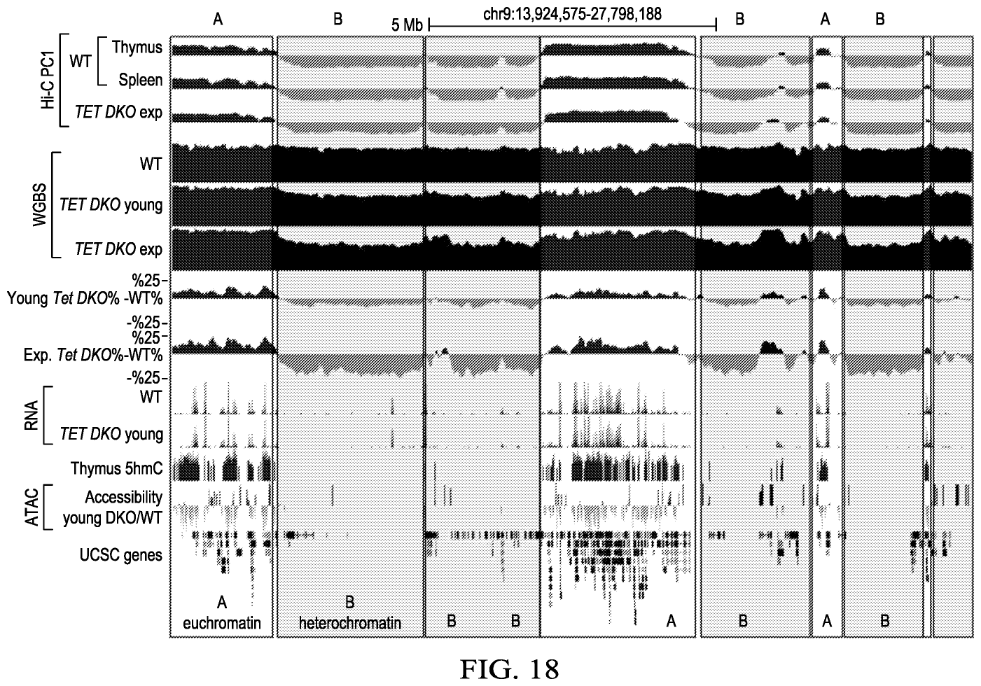

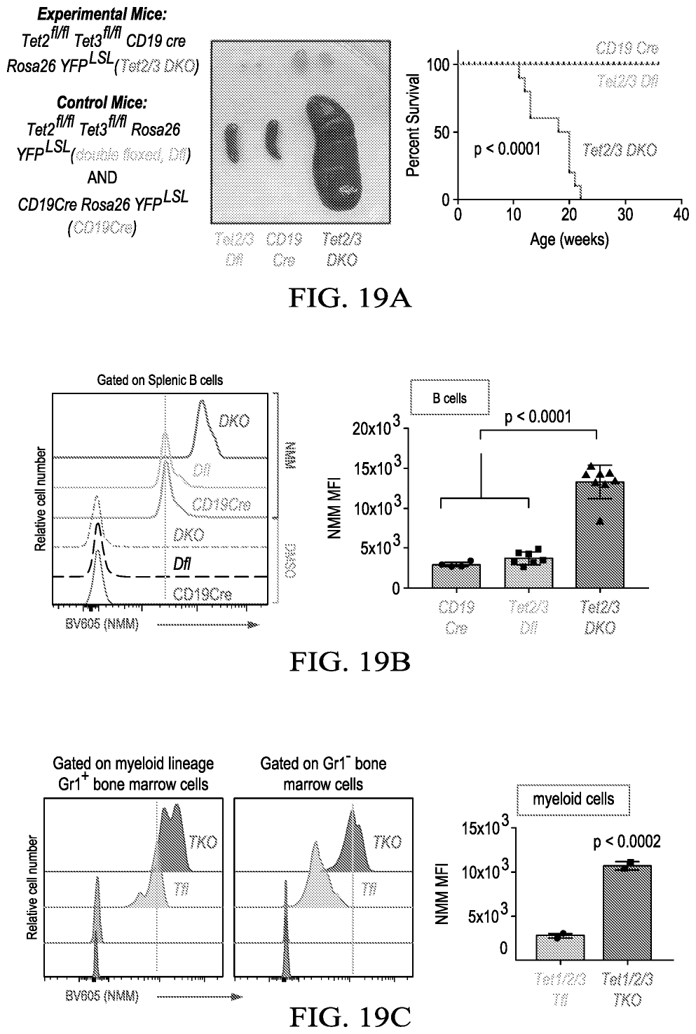

Example 5: Genomic features of expanded TET-deficient cells. The genomic features of expanded TET-deficient NKT, B and myeloid cells have been examined. All three types of expanded TET-deficient cells exhibited increased clonality, a characteristic feature of hematopoietic malignancies, based on T cell and B cell receptor junctional region sequencing and barcode sequencing (FIG. 17). They also displayed DNA hypomethylation in the heterochromatic compartment, shown for NKT cells in FIG. 18, and several features of heterochromatin dysfunction: (a) increased DNA damage; (b) increased levels of R-loops detected by DNA dot blot and by flow cytometry with the S9.6 antibody against RNA:DNA hybrids; (c) increased levels of G-quads detected with the G-quad binding dye N-methylmesoporphyrin IX (NMM) (FIG. 19); (d) activation of transposable and repeat elements, including LINE and LTR elements which are primarily located in heterochromatin. (e) The NKT cells also showed accumulation of single nucleotide polymorphisms, mostly in the heterochromatic compartment; and (f) increased propensity to CNAs and aneuploidies.

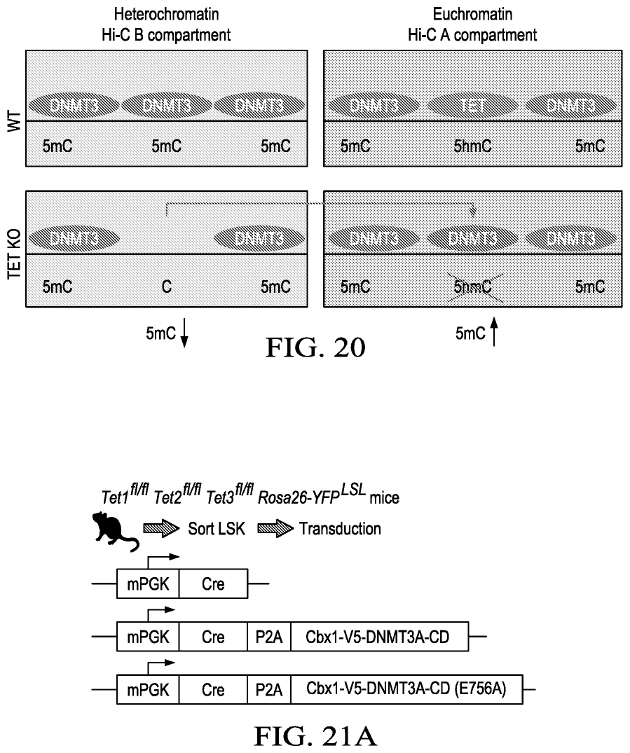

Example 6: Potential mechanism by which TET deficiency results in heterochromatic DNA hypomethylation. The inventors have shown, through analysis of published data from other labs, that TET1 occupied only euchromatic regions in WT mESC, but that in the absence of TET1, DNMT3A showed a striking relocalization to the regions previously occupied by TET1 in the parental WT ESC (see model of FIG. 20).

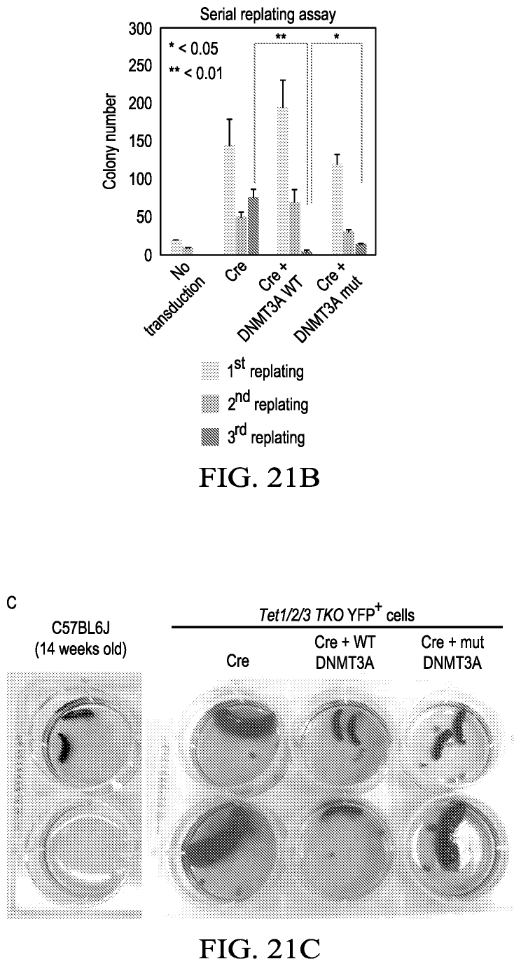

Example 7: To test the hypothesis shown in FIG. 20, the inventors directed the catalytic domain (CD) of DNMT3A to heterochromatin (FIGS. 21A to 21C) using a modified version of the system shown in FIG. 17. The inventors designed a lentiviral expression plasmid in which cDNA for Cre recombinase was separated by a P2A sequence from cDNA encoding a fusion protein of HP1b (heterochromatin-binding protein 1, encoded by Cbx1) with the catalytic domain (CD) of DNMT3A (FIG. 21A). Bone marrow or HSPC (LSK cells) from Tet1/2/3 triple foxed mice bone marrow chimeras were transduced with the lentiviral plasmids, and used either in serial replating assays or for transfer into irradiated recipient mice. Notably, a complete rescue by the WT HP1b-DNMT3a fusion protein in both assays was observed (FIGS. 21B, 21C). Whereas LSK cells transduced with Cre alone (and therefore TET-deficient) showed increased serial replating compared to untransduced control cells (FIG. 21B, compare clusters 1 and 2), cells transduced with the Cre-P2A-HP1b-DNMT3A WT expression plasmid progressively lost serial replating capacity, exactly like the untransduced control cells (FIG. 21B, compare clusters 1, 3 and 2, 3). Similarly, when the mice were sacrificed at 70 days after transfer, mice that received Cre-transduced cells had developed splenomegaly (spleen weights 594 and 883 mg in replicate mice, a third mouse still to be analyzed but clearly showing signs of sickness), whereas spleen weights for WT mice were 93±5.5 mg and for mice that received cells transduced with HP1b-DNMT3A (WT), 91+26 mg (FIG. 21C; n=3, mean+SD in each case).

As a control, the inventors generated the same HP1b-DNMT3A expression plasmid with a single point mutation (E756A) in the DNA/11′3A cDNA that was expected to impair, if not abrogate, DNA methyltransferase activity (FIG. 21A). Since the argument was that restoration of DNA methylation to heterochromatin was necessary for rescue of the biological defects in TET-deficient cells, it was expected that cells expressing this mutant protein would behave similarly to Cre-transduced, TET-deficient cells. While some rescue was observed, the effect was intermediate (FIG. 21B, compare clusters 2, 3, 4). In the in vivo assay (FIG. 21C), spleen weights were 495, 87 and 87 mg, indicating incomplete penetrance of rescue—either because of incomplete loss of catalytic activity or because the rescue depends on both the catalytic activity and some scaffold function of DNMT3A.

Example 8: The inventors found that increasing DNMT activity in heterochromatin rescues the loss of DNA hypomethylation and as well as the biological dysfunction observed in aging, cell senescence, clonal hematopoiesis and other selected premalignant and malignant syndromes.

The inventors have mapped 5mC, 5hmC and other oxi-mCs using whole-genome bisulfite sequencing (WGBS), CMS-IP and PacBio SMRT sequencing and have examined the genomic locations of numerous transcription factors and transcriptional regulators (NFAT, CTCF, TET, DNMT) by chromatin immunoprecipitation (ChIP)-sequencing and CUT & RUN, as well as native ChIP of non-crosslinked, MNase-digested DNA for histone modifications. They have monitored the expression of transposable elements and have performed whole genome sequencing (WGS) and low coverage single-cell WGS for aneuploidies (FIG. 23). The inventors have also established out methods to map both R-loops and G-quads; to map R-loops the inventors have used MAPR, a method related to CUT & RUN that diffuses a catalytically inactive RNase H fused to MNase into immobilized cell nuclei to release R-loops for sequencing (active RNase H very selectively cleaves the RNA strand in RNA:DNA hybrids, but the catalytically inactive enzyme binds R-loops strongly enough to permit their immunoprecipitation and sequencing). Finally, the inventors have mapped G-quads using a single-chain antibody that recognizes G-quads (BG4) fused to an immunoglobulin constant region. The inventors are have determined how newly formed R-loops and G-quads in TET-deficient relative to WT cells are distributed between euchromatic and heterochromatic genome regions, and whether their formation or resolution is affected by changes in DNA modification attributable to TET and DNMT. The inventors have developed a system to target DNMTs to heterochromatin, allowing the restoration of DNA methylation levels in heterochromatin of aged and transformed cells and reverse heterochromatin dysfunction (FIG. 21A-21C).

Example 9: The inventors investigated heterochromatin dysfunction as a function of both cellular age and number of cell divisions, using multiple cellular systems: IMR90 fibroblasts, freshly isolated CD4+ and CD8+ T cells from young and old human donors, and commercially available CD34⁺ cells (enriched for hematopoietic precursor cells) from human newborns (cord blood) and older adults. The inventors determined, by 5hmC mapping by CMS-IP (using spike-ins to estimate absolute 5hmC levels), whether the hypomethylation observed with cell division and/or aging is due to TET loss-of-function, and established that time to senescence can be delayed, and hypomethylation can be rescued with Vitamin C, an activator of TET proteins and other Fe(II) and aKG-dependent dioxygenases.

Tet1/2/3 triple-foxed (Tet1/2/3 Tfl) RosaYFP^{LSL} or RosaYFP^{LSL} mice were used, left treated or treated with tamoxifen; (ii) DNMT3a (or multiple Dnmt) foxed mice, also bearing the RosaYFP^{LSL} reporter and left treated or treated with tamoxifen. Mouse fibroblasts have a longer passage time to senescence, so IMR90 human fibroblasts in which partial TET or DNMT loss-of-function was induced by genome editing were also used.

Example 10: The inventors determined if the senescent or multiply divided cells show relocalisation of DNMT3A away from heterochromatin compared to early passage/early cycling cells; and whether transduction with the HP1-DNMT3A fusion protein delays the onset of senescence. The inventors have shown that loss of DNA methylation is also rescued (by WGBS or amplicon-based BS-seq in selected heterochromatic versus euchromatic regions), and using a mutant DNMT3A with multiple mutations expected to impair catalytic activity, determined whether DNMT catalytic activity or scaffold function are required. The inventors delayed senescence using all three HP1-DNMT fusion proteins (HP1a, b and g, DNMT1, 3A and 3B) as well as all three full-length DNMTs; using HP1b alone as a control.

Example 11: The inventors assessed various features of heterochromatin dysfunction in each of these systems, as warranted: (i) activation of transposable and repeat elements (TE, RE) by total RNA-seq; (ii) assessment of changes in R-loops and G-quads by flow cytometry, DNA dot blot and immunoprecipitation as discussed above; (iii) assessment of CNAs and aneuploidies by metaphase spreads and/or low-coverage bulk or single-cell WGS (FIG. 23A-23B); (iv) assessment of telomere function in human cells; and (iv) assessment of the fraction of SNVs in heterochromatic versus euchromatic regions. Where CNAs and/or aneuploidies are observed, the inventors assessed centromere dysfunction by imaging cell divisions (FIG. 23A-23B); and tested the RPE retinal pigment epithelial cell line, expressing H2B-GFP for ease of imaging, at different passage numbers in culture. As controls for these imaging experiments, the inventors used RPE-H2B-GFP cells in which partial TET or DNMT loss-of-function has been induced by genome editing.

Example 12: Clonal hematopoiesis (CH). The inventors established that ASXL1, Lamin A and SRSF2 mutations, all associated with CH, involve varying degrees of heterochromatin dysfunction.

(a) ASXL1. The major mutation observed in CH is in DNMT3A (>50%), followed by mutations in TET2 and ASXL1 (8-9%). The DNA/11′3A and TET2 mutations are loss-of-function, whereas ASXL1 mutations appear to be gain-of-function as judged by increased deubiquitylation activity of the ASXL1-BAP1 complex for H2AK119Ub. The inventors have imported the Dnmt3a fl/fl and ASXL1 truncation knock-in (Asxl1-KI) mice, and crossed them to Rosa26-YFP^{LSL}, Cas9 transgenic and Tet2 fl/fl mice. HSPC from Asxl1-KI mice show increased serial replating capacity. The inventors assessed features of heterochromatin dysfunction in each of these systems as described above, monitoring (i) in vivo biological phenotypes, (ii) self-renewal in serial replating assays, and (iii) DNA methylation in heterochromatin and euchromatin, in HSPC (LSK) cells from Asxl1-KI and Asxl1-Wet2−/− mice, and Asxl1-KI, Dnmt3a fl/fl Mx1Cre mice after polyI:polyC treatment. The inventors used the barcoding assay shown in FIG. 18 to monitor (iv) changes in clonality; (v) TE and RE activation; (vi) changes in R-loops and G-quads; and (vii) CNAs and aneuploidies by low-coverage WGS on bulk populations or single cells. (viii) Where increased clonality was observed, the inventors assessed the fraction of SNVs in heterochromatic versus euchromatic regions. (ix) Where CNAs and/or aneuploidies were observed, the inventors generated the ASXL1 gain-of-function mutation in the H2B-GFP⁺ RPE cell line, so as to assess centromere dysfunction by imaging cell divisions (see FIG. 23A, 23B). These experiments showed there is an exacerbation of the biological phenotypes of Asxl1-KI,Tet2−/− mice and Asxl1-KI, Dnmt3a fl/fl Mx1Cre mice after polyI:polyC treatment, over those of the individual mutant mice. They also showed that the increased serial replating ability of HSPC from Asxl1-KI mice stems from decreased DNA methylation in heterochromatin or alternatively, from interference with a different pathway that may or may not be associated with heterochromatin dysfunction.

(b) Lamin A, a protein required for localization of heterochromatin at the nuclear periphery, is highly downregulated in HSC from older individuals. The inventors set up systems for CRISPR-Cas9-sgRNA-mediated disruption of LMNA (encoding Lamin A) in human CD34⁺ cells, and interrogated the features of the resulting cells as described above (controls: cells expressing Cas9 with a control sgRNA).

(c) Splicing factors (focus on SRSF2). More than 50% of cases of myelodysplastic syndrome (MDS) show recurrent mutations in genes encoding splicing factors, including SF3B1, U2AF1, ZRSR2, and SRSF2; the mutations are invariably heterozygous and mutually exclusive, consistent with the idea that the wildtype proteins are needed to carry out their primary functions in splicing. SF3B1 and SRSF2 are also frequently mutated in clonal hematopoiesis, although to a lesser extent than TET2 and ASXL1 (˜1.5-2.5%). The inventors focused on the splicing factor SRSF2 because (i) it is commonly mutated, displaying recurrent hotspot mutations at P95 (to H, L, R, T etc) in clonal hematopoiesis, MDS and certain other cancers; and (ii) expression of mutant SRSF2 P95H in hematopoietic and non-hematopoietic cell lines results in increased R-loops.

Srsf2 (P95H) knock-in mice exhibit multi-lineage dysplasia of the hematopoietic system. The inventors analyzed them either individually or after crossing them to Tet2−/− or Dnmt3a fl/fl Mx1Cre mice, and extended the experiments to mice bearing multiple mutations of Srsf2 (heterozygous P59H), Asxl1 (gain-of-function truncation), Tet2 and Dnmt3a. The inventors also expressed mutant Srsf2 (P95H) in mouse HSC and human CD34⁺ cells, using a Dox-inducible lentiviral vector that allows expression of exogenous epitope-tagged SRSF2 while at the same time knocking down expression of endogenous SRSF2.

Example 13: Role of heterochromatic DNA hypomethylation and heterochromatin dysfunction in other malignancies. The experiments for this section were performed in the H2B-GFP⁺ RPE cell line (using low passage cells confirmed to be karyotypically normal) so that the same cells can be used to measure centromere dysfunction by imaging above. Note that these experiments are also related to cellular senescence and aging, as heterochromatic DNA hypomethylation, heterochromatin dysfunction and centromere dysfunction are monitored as a function of cell passage number. The results show heterochromatin dysfunction is indeed observed, and its occurrence is associated with or independent of DNA methylation or centromere dysfunction.

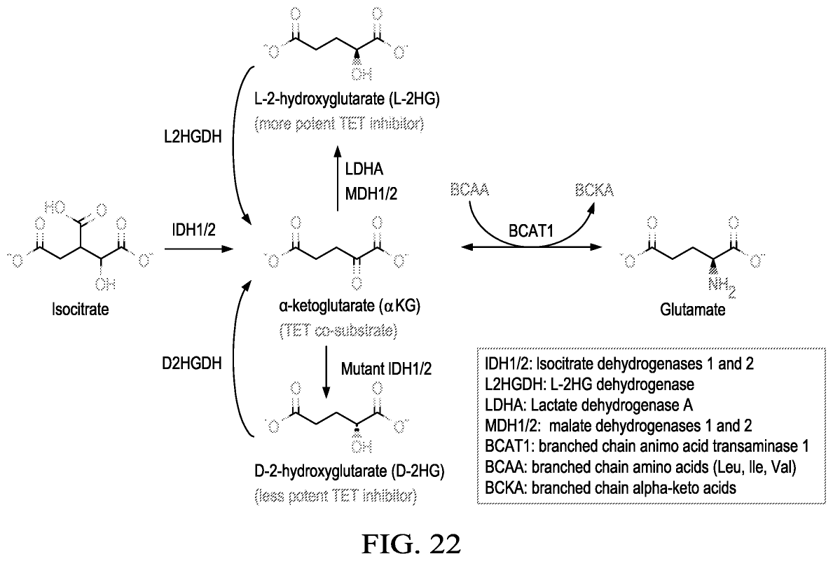

Example 14: Conditions potentially associated with TET loss-of-function. TET loss-of-function can occur even in the absence of any coding or splice junction mutations in TET genes through alterations in promoter methylation or post-transcriptional or post-translational process such as microRNAs and E3 ligases. The inventors focused on metabolic alterations that increase the levels of 2-hydroxyglutarate (2HG), a competitive inhibitor of aKG, or decrease the levels of the substrate aKG itself (FIG. 22). Specifically, the inventors examined (i) recurrent gain-of-function mutations in IDH1 and IDH2, observed in acute myeloid leukemia (AML) and glioblastoma; the mutant IDH enzymes overproduce a normally minor metabolite, D-2HG, aka R(−)-2HG, which is a relatively potent inhibitor of TET proteins and other Fe(II) and 2OG-dependent dioxygenases, and the methylation “signatures” of IDH-mutant tumors are similar to those of tumors with TET mutations. (ii) The inventors also examined loss of function mutations in L-2GH dehydrogenase (L2HGDH), observed in diffuse large B cell lymphoma (DLBCL) and certain renal cancers, which result in greatly increased levels of L-2HG, aka S(+)− 2HG, an even more potent inhibitor of Fe(II) and 2OG-dependent dioxygenases including the TET enzymes. (iii) Finally, the inventors examined the effects of overexpression of the branched chain amino-acid transaminase BCAT1, that often shows increased expression in AML and whose overexpression is associated with decreased aKG (FIG. 22).

Example 15: Conditions associated with heterochromatin dysfunction without obvious relation to TET loss-of-function. (i) BRCA1 generates H2AK119Ub whereas the ASXL1/BAP1 deubiquitinase complex removes it; ASXL1 gain of function mutations and BRCA1 loss-of-function mutations have similar effects on cellular function, although in different cancer types. (ii) ATRX, a known G-quad binding protein, seems to protect G-quads from being resolved, because ATRX knockouts show increased G-quads, increased replication stress, CNAs, chromosome breaks and DNA damage, and increased sensitivity to G-quad stabilizing agents.

The experiments described in Examples 14 and 15 are essentially identical to those described for ASXL1, Lamin A and SRSF2 above, except that they were performed in RPE cells. Overexpression of BCAT1 and mutant IDH1/IDH2 were performed using standard expression; depletion of L2HGDH, BRCA1 and ATRX were performed by CRISPR/Cas9- mediated genome editing. In each case, the inventors tested for all the readouts described above; where DNA hypomethylation was involved, the inventors determined whether reintroduction of full-length or HP1b-targeted DNMTs rescues the aberrant phenotypes observed. Since strategies to increase DNMT activity are potentially therapeutic, the inventors determined which mutant phenotypes are accompanied by, and which are independent, of heterochromatin DNA hypomethylation, and which might be ameliorated by (targeted) increases of DNMT activity. The inventors also considered limited CRISPR/Cas9 or CRISPRa (CRISPR activation) screens in which selected sgRNAs were introduced into mutant (e.g. double Tet2/Dnmt3a-deficient or Tet1/2/3 TKO) cells already rescued via expression of the HP1b-Dnmt3a fusion proteins, to identify candidates whose overexpression or depletion interferes with rescue. Currently, the opposite strategy of using hypomethylating agents (5-azacytidine, decitabine) appears to show clinical efficacy in MDS; these agents seem to act by potentiating the interferon response and improving immune responses through reactivation of endogenous retroviruses, a characteristic feature of the heterochromatin dysfunction (due to genome-wide DNA hypomethylation) that is caused by demethylating agents. The strategy of improving DNMT function may also be promising, however, especially for premalignant conditions.

Example 16: Localisation of DNMT3A in euchromatin and heterochromatin—identification of interacting partners. In this section, the inventors establish, why in the absence of TET1 (specifically Tet1 deletion in mESC), DNMT3A prefers to occupy the same regions that TET1 previously occupied before its deletion (FIG. 20). It is well established that H3K36 methylation (H3K36me2 and me3, respectively) are important for the recruitment of DNMT3A and DNMT3B to genomic regions. The inventors used both mESC and somatic cells for these experiments; the original observations were obtained in mESC but DNA methylation/demethylation dynamics are very different in mESC versus somatic cells. The inventors used the same cell lines that were utilized above: (i) TET iTKO mESC in which all three TET proteins can be inducibly deleted, leading to the rapid appearance of aneuploidies within 5 days; and (ii) karyotypically normal RPE cells. The inventors generated Turbo-ID fusion proteins of DNMT3A and TET1, introduced them into the TET iTKO mESC, and confirmed (by ChIP-seq) that they localise with the endogenous proteins. The inventors also deleted all three endogenous TET proteins by a short treatment with 4-OHT, after which proteins in the vicinity of either DNMT3A or TET1 were labelled briefly by addition of exogenous biotin. Nuclear proteins were pulled down using streptavidin beads and subjected to mass spectrometry. By comparing “interacting” protein profiles for DNMT1-TurboID in the presence or absence of TET1, the inventors identified proteins that are selectively present in the vicinity of the regions where DNMT relocalizes in the absence of TET1.

By doing the same for TET1-TurboID in TET floxed or TET-deleted mESC, the inventors identified proteins that are selectively present in the vicinity of the regions where TET1 binds. The overlap between the “interacting” protein profile selective for DNMT1-TurboID in the absence of TET1, and the profiles for TET1-TurboID itself, inform about the potential common interacting partners for TET1 and DNMT3A. The inventors depleted these partners using CRISPR/Cas9, and interrogated the effects on localisation of TET1 and DNMT3A (by ChIP-seq) and 5mC and oxi-mC distribution (by WGBS and pyridine-borane sequencing). As an alternative approach, the inventors directed a dCas9-APEX fusion protein to sites that are already known to occupy in a mutually exclusive manner by DNMT3A and TET1 in euchromatin of mESC. These experiments were repeated in RPE cells (which express mainly TET2 and TET3), after examining TET and DNMT3A localisation and 5mC and oxi-mC distribution patterns in these cells.

Example 17: Relation of DNA hypomethylation to centromere dysfunction. Centromeres are located in heterochromatin, and many cancers are characterized by aneuploidies and chromosomal trans-locations. The inventors observed a recurrent chromosome 17 trisomy, as well as other partial aneuploidies, in each of 12 samples of Tet2/3 DKO NKT cells that had expanded after secondary transfer into immunocompetent recipient mice (FIG. 23). More pertinently for the study of mechanism, the inventors observed that acute deletion of all three TET proteins in ES cells resulted in the rapid appearance (within days) of aneuploid cells and chromosome segregation defects. The inventors used these TET iTKO cells to establish whether the rapid onset of aneuploidies can be reversed by heterochromatin-targeted DNMTs (FIG. 21).

Example 18: The inventors examined the relationship between chromosome segregation defects, propensity to aneuploidies and DNA modification status in centromeric regions. The only known sequence-specific DNA-binding protein in the inner kinetochore complex is CENP-B, whose consensus motif contains two essential CpG sequences with seven nucleotides (CCCGNNTNNNNCGAA, including an essential AT base pair) between them. The inventors have already shown in EMSA assays with recombinant CENP-B that CENP-B prefers both CpGs to be fully unmodified in binding assays in vitro. Moreover, analysis of 5hmC and 5mC levels in centromeric sequences (from published TAB-seq data on mESC, using the complete sequence of a specific fosmid overlapping a centromeric region as reference) indicated that the levels of both these modified bases are decreased in TET-deficient cells. CENP-B stabilizes the binding of CENP-C, a major centromere protein required for kinetochore assembly, and loss of CENP-B in RPE cells decreased the fidelity of chromosome segregation, especially for neocentromeres and the Y chromosome which lack sequence motifs for CENP-B DA binding. These findings, as well as the propensity of DNMT-deficient cells to aneuploidies, show that the DNA modification status of centromeres is important for faithful chromosome segregation.

Example 19: The inventors used hybridization capture of CENP-B-containing centromeric regions to identify all cytosine modifications in the 120 bp minor satellite repeats in mouse centromeres that contain the 17 bp CENP-B site. First, the inventors treated the captured DNA with a recombinant TET protein that has been mutated to be far more efficient at converting all modified cytosines (5mC, 5hmC, 5fC) to 5caC; the DNA was then adapter-ligated and long DNA fragments sequenced without amplification using the PacBio platform, which detects 5caC directly.

The inventors also examined the transcription of centromeric sequences, based on suggestions that CENP-B may behave as a transcription factor, promoting transcription of minor satellite repeats in the mouse. The inventors connected the levels of these minor satellite repeat transcripts to DNA modification status and CENP-B binding in 4-OHT-treated and untreated TET iTKO mESC.

WGBS, Hi-C and RNA-seq library preparations were performed as previously described.

External data. The external data was downloaded from Gene Expression Omnibus (GEO) and the European Nucleotide Archive (ENA). See Table S1-S2 for details on datasets.

WGBS mapping and analysis. The inventors employed BSMAP (v2.9) (1) to align reads from bisulfite-treated samples to the mm10 mouse reference genome allowing 4 mismatches. Reads mapping to multiple locations in the reference genome with the same mapping score were removed as well as the 5′ ends bearing quality lower than 20 (mapping parameters: −n 1 −v 4 −w 2 −r 0 −q 20 −R −p 8). Single and paired-end reads were mapped as appropriately.

Duplicate reads caused by PCR amplification were removed by BSeQC (v1.0.3) (2) using default parameters. An effective genome size of 1.87e9 (as suggested in BSeQC for Mus musculus genome) was employed to calculate maximum coverage at the same genomic location. In addition, BSeQC was employed for removing DNA methylation artefacts introduced by end repair during adaptor ligation. For paired-end sequencing, overlapping segments of two mates of a pair were reduced to only one copy to avoid considering the same region twice during the DNA methylation quantification.

To estimate CpG DNA methylation, the inventors employed the methratio.py tool included in BSMAP (v2.9) (1), merging DNA methylation at each CpG di-nucleotide (combining CpG methylation ratios on both DNA strands). The inventors required each CpG to be covered by at least 5 reads (merging biological replicates) in order to be considered in the downstream analysis. Only CpGs within the autosomes were considered for the analysis (no sex chromosomes included). For the window analysis and the integration with Hi-C data, the inventors only considered for the analysis 1 kb windows with at least 3 CpGs, and 10 kb windows with at least 10 CpGs.

Hi-C mapping and analysis. Reads corresponding to each extreme of a fragment were trimmed after the corresponding restriction site (e.g. MboI in the case of the NKT datasets) using HOMER (3) homerTools trim and independently mapped employing BWA-aln (v0.7.13) (single-end mode) (4). Reads were filtered out if they had a MAPQ score of less than 30, and only reads that were at least 25 bp were considered for the rest of the analysis. Only reads falling within the autosomes were considered for the analysis (no sex chromosomes included).

Hi-C analysis was performed using HOMER (3) and its Hi-C data analysis suite. Independently-mapped reads were paired using the makeTagDirectory command, allowing only 1 tag per bp (−tbp 1). Reads were filtered to remove uninformative reads (contiguous genomic fragments, self-ligation, re-ligation, and reads originating from regions of unusually high tag density) and also filtered based on the distance tor restriction sites (—genome mm10—removePEbg—restrictionSite GATC—both—removeSelfLigation—removeSpikes 10000 5).

To perform the principal component analysis (PCA) of Hi-C data (A/B compartment identification), the inventors used the tool runHiCpca.pl on the normalized interaction matrix, with the options—res 50000—superRes 100000—genome mm10. For analysis involving the Hi-C A/B compartments (e.g. integration with WGBS data), only the bins associated to the same Hi-C compartment in all biological replicates (of a given sample) were considered in the analysis.

CMS-IP and TAB-seq mapping and analysis. CMS-IP data were mapped in a similar way to WGBS. Signal per 1 kb window (log₂ enrichment over input) was computed using MEDIPS (Bioconductor package) (5), using the functions MEDIPS.createSet (with the options extend=300, shift=0, window_size=1000, BSgenome=“BSgenome.Mmusculus.UCSC.mm10”, uniq=1e-5, paired—F for single-end data; extend=0, shift=0, window_size=1000 BSgenome=“BSgenome.Mmusculus.UCSC.mm10”, uniq=1e-5, paired-1′ for paired-end data) and MEDIPS.meth (with the options p.adj=“bonferroni”, cliff. method=“edgeR”, minRowSum=10, diffnorm=“tmm”) for statistical comparisons. TAB-seq data were processed in a similar way to WGBS data.

ChIP-seq and ATAC-seq mapping and analysis. ChIP-seq and ATAC-seq data were mapped employing BWA v0.7.13 (4). Depending on the read length and sequencing type, BWA-aln was used in single or paired-end mode to map reads that were shorter than 70 bp, and reads with length>=70 bp were mapped using BWA-mem. In both cases, Mus musculus genome (mm10 downloaded from UCSC website) was used as reference. Reads were filtered out if they had a MAPQ score of less than 30, and only reads that were at least 25 bp were considered for the rest of the analysis. Only reads falling within the autosomes were considered for the analysis (no sex chromosomes included). For differential enrichment or occupancy analysis, the signal per 1 kb genomic window was computed using MEDIPS (Bioconductor package) (5), using the functions MEDIPS.createSet (with the options extend=300, shift=0, window_size=1000, BSgenome=“BSgenome.Mmusculus.UCSC.mml 0”, uniq=1 e-5, paired=F for single-end data; extend=0, shift=0, window_size=1000 BSgenome=“BSgenome.Mmusculus.UCSC.mm10”, uniq=1e-5, paired-7′ for paired-end data) and MEDIPS.meth (with the options p.adj=“bonferroni”, cliff. method=“edgeR”, minRowSum=10, diffnorm=“tmm”) for statistical comparisons.

Replication timing and Lamina B data. Processed data for replication timing (6, 7) was downloaded from https://www2.replicationdomain.com/

RNA-seq mapping and transposable element (TE) analysis. Quality and adapter trimming was performed on raw RNA-seq reads using TrimGalore! 431 v0.4.5 (http://www.bioinformatics.babraham.ac.uk/projects/trim_galore/) with default parameters, retaining reads with minimal length of 25 bp. Ribosomal RNA reads were filtered out using Tagdust2. Resulting reads were aligned to mouse genome mm10 using STAR v2.5.3a (8)(Dobin et al., 2013) with alignment parameter—outFilterMismatchNmax 4—outFilterMultimapNmax 100—winAnchorMultimapNmax 200.

The inventors employed TETranscripts (9) to quantify gene and transposable element transcript abundances. This program proportionally assigns read counts to the corresponding gene or transposable element. The inventors used this package on mode-multi to be able to use ambiguously mapped reads to perform the differential expression analysis. The inventors used the transcript annotations of the mouse genome mm10, and the repeat element annotation from UCSC RepeatMasker track of mouse genome mm10.

The DESeq2 package v1.14.1 (10) was used to normalize the raw counts and identify differentially expressed genes or transposable elements (FDR cutoff of p<0.1). Genes or repeat elements with less than 10 reads total were pre-filtered in all comparisons as an initial step. For total (ribodepleted) RNA-seq sample analysis, the highest expressed genes were used as control genes for size factor estimation in DESeq2. For polyA+ RNA-seq sample analysis, p-values of two independent experiments (same biological conditions, different library preparation methods, TruSeq and SMARTseq) were combined using the Fisher method, as implemented in the R package metaRNASeq (https://cran.r-project.org/web/packages/metaRNASeq)

Whole-genome sequencing (WGS) mapping. WGS libraries were sequenced on the Illumina Hiseq 2500 using paired-end reads at a >20× coverage per sample. Adapters and low-quality bases were trimmed before mapping, and reads with length>=70 bp were mapped to the Mus musculus genome (mm10 downloaded from UCSC website) using BWA-mem (4) with default options. Optical duplicate reads were removed Picard MarkDuplicates tool.

Tumor-specific variant calling. Following GATK (11) best practices for variant detection, additional pre-processing steps including recalibration of base quality scores were performed prior to variant detection. MuTect2 (12) somatic variant caller was employed to identify single-nucleotide variants (SNVs), using matched (samples Mouse A, B and C) tail information as normal (non-tumor tissue), as well as a panel of mutations observed in the recipient B6.SJL-PtprcaPep3bBoyJ mice (recipient mouse strain). In order to avoid false detection of tumor-specific SNVs (false positives), the variant calling process was repeated in a pairwise manner using the unmatched tails as normal (e.g. Tail B and C for Mouse A), and only SNVs detected in all three comparisons were included in the analysis. SNV filtering was performed using MuTect2 (12) default parameters. Mutational signature analysis was performed with Bioconductor's package MutationalPatterns (13). ANNOVAR (14) was used to perform functional annotation of mutations (synonymous, nonsynonymous, frameshift and nonsense mutations).

TCR repertoire analysis. The overlapping paired-end reads (250×250) were merged into a single longer read, and ClonotypeR (18) was employed to detect clonotypes in the sequence reads, extract the CDR3 sequences and quantify TCR repertoire abundances.

Mice. Mice were housed in a pathogen-free animal facility at the La Jolla Institute. They were used according to protocols approved by the Institutional Animal Care and Use Committee (IACUC). Tet2−/− mice were generated by crossing CMVCre mice to Tet2fl/fl mice, in which exons 8, 9 and 10 that code for the catalytic HxD domain, were floxed (flanked by LoxP sites) (15). Tet3fl/fl mice were generated by targeting exon 2 (16) Tet2−/− and Tet3fl/fl mice were crossed with CD4Cre (17) mice to generate Tet2−/− Tet3fl/fl CD4Cre mice (DKO mice). The Tet2/3 DKO mice are in the C57BL/6 background. B6.SJL-PtprcaPep3bBoyJ (CD45.1⁺) mice, C57BL/6 (CD45.2³⁰) mice were purchased from Jackson laboratory (B6(C)-Cd1d1tm1.2Aben/J). Both male and female mice were used in this disclosure with similar findings. Invariant NKT cells were isolated from young mice (3-4 weeks old). The recipients were of the same sex as the donors. Both male and female recipients were used and similar results were obtained.

Flow Cytometry associated with NKT cell experiments. Cells were isolated from thymus, spleen, lymph nodes and bone marrow. Surface staining was performed using antibodies from Biolegend and eBioscience: CD4 (RM4-5), CD8 (53-6.7), TCRb (H57-597), B220 (RA3-6B2), CD45.1 (A20), CD45.2 (104). TCRVb2 (B20.6), TCRVb 5.1, 5.2 (MR9-4), TCRVb7 (TR310), TCRVb8.1, 8.2 (MR5-2) were purchased from BD Pharmingen. aGalCer-CD1d tetramer was obtained from the NIH Tetramer Core. Vα14i NKT cells were routinely defined as TCRb intermediate, B220-negative and positive for aGalCer-CD1d tetramer binding. For the pH2Ax staining the Alexa Fluor 647 anti-H2Ax-Phosphorylated (Ser139) (clone 2F3)(Biolegend) was used. Acquisition was performed in a BD LSR Fortessa (BD Biosciences) using the BD FACSDiva Software. Data analysis was performed with FlowJo (Treestar).

Isolation of Vα14i NKT cells. Vα14i NKT-cell preparations for adoptive transfer and DNA isolation experiments were performed using in case of control mice a pool of cells (isolated from thymus or spleen as indicated on each case) from C57BL/6 mice and from age- and sex-matched DKO mice. For fluorescence-activated cell sorting (FACS), cells from wild type mice were depleted of CD19⁺ (6D5), TER-119⁺ (TER119), CD8⁺ (53-6.7), CD11c⁺ (N418), F4/80⁺ (BM8) and CD11b⁺ (M1/70) cells using biotinylated antibodies (Biolegend) and subsequent binding to magnetic streptavidin beads (Life Technologies). The unbound cells were incubated with 1 mg/ml Streptavidin A (Sigma Aldrich) and subsequently stained with aGalCer-loaded CD1d tetramers and anti-TCRβ, after which tetramer-binding, TCRβ⁺ cells were isolated using a FACSAria cell sorter (BD Biosciences). To obtain DKO cells, no depletion was performed since NKTs are massively expanded. Rather, B220-, tetramer-binding, TCRβ+ cells were isolated using a FACSAria cell sorter (BD Biosciences).

Adoptive transfer experiments. NKT-sorted cells were transferred retro-orbitrally to non-irradiated, fully immune-competent congenic (B6.SJL-PtprcaPep3bBoyJ) (CD45.1⁺) mice.

TCR repertoire sequencing. Vα14i NKT cells were isolated by FACS from wild type and Tet2/3 DKO young mice or were magnetically purified by recipients of Tet2/3 DKO NKT cells. RNA was isolated with the E.Z.N.A. HP Total RNA kit (Omega) according to the manufacturer's instructions. cDNA was prepared using Superscript III (Invitrogen). Subsequently, PCR was performed for amplification of the gene segments with specific forward primers (sequences shown below) for Vb8.1 (primer MuBV8.1N), Vb8.2 (primer MuBV8.2N) and Vb8.7 (primer MuBV7) regions and a reverse primer for the b chain constant region (primer MuTCB3C). Amplicons were quantified and pooled using HS Qubit (Life Technologies). Adaptors (NEB) were ligated and libraries were amplified using Kapa HiFi (Kapa Biosy stems). Amplified libraries were quantified using HS Qubit, their size was evaluated using Bioanalyzer and sequenced in an Illumina Miseq.

| | | SEQ ID NO: |
|---|---|---|
| MuBV8.1N | GGC TGA TCC ATT ACT CAT ATG TC | 1 |
| MuBV8.2N | TCA TAT GGT GCT GGC AGC ACT G | 2 |
| MuBV7 | TAC AGG GTC TCA CGG AAG AAG C | 3 |
| MuTCB3C | AAG CAC ACG AGG GTA GCC T | 4 |

Whole-genome bisulfite sequencing (WGBS) Library preparation. Vα14i NKT cells were isolated by flow cytometry and DNA was isolated using the PureLink genomic DNA mini kit (Life technologies). DNA was fragmented. 1.5 μg of the fragmented DNA was used for the library preparation and bisulfite treatment was done as described in ref 26. After the bisulfite conversion the purified DNA was amplified for 4 cycles (low amplification) using Kapa HiFi Uracil+ (Kapa Biosystems). 2 independent WGBS samples per genotype were evaluated.

Whole Genome Sequencing (WGS) Library preparation. Genomic DNA was isolated from purified NKT cells using the PureLink genomic DNA mini kit (Life technologies). DNA was fragmented to an average size of 400 bp using the Adaptive Focused Acoustics Covaris S2 instrument. Libraries were prepared using the TruSeq DNA PCR-Free Sample Preparation kit (Illumina) according to the manufacturer's guidelines. Libraries were purified, pooled according to the instructions of the manufacturer and sequenced in an Illumina HiSeq 2500 instrument.

Hi-C Library preparation. Between 0.6 and 1.5×10\hat{ }6 NKT cells were fixed in complete medium containing 1% Formaldehyde, then quenched with 125 mM glycine and washed twice with an excess of PBS. Cells were then resuspended in lysis buffer containing 0.5% SDS and lysed at 62° C. for 7 minutes. This step also allows to remove proteins that were not fixed to the chromatin. SDS was further quenched with 1% Triton-X-100 at 37° C. for 15 minutes. Next, permeabilized nuclei were reacted with 100 units of MboI overnight at 37° C. After subsequent washing and inactivation of MboI, the restriction sites were further filled in with Biotin-14-dATP and Klenow polymerase at room temperature for 40 minutes. Samples were transferred into a ligation solution containing 600 units of T4 DNA ligase. Proximity ligation was stopped by addition of 2-fold molar excess of EDTA, and samples were decrosslinked at 65° C. for 16 h00. DNA was further purified by proteinase K digestion and phenol/chloroform extraction. For library preparation, 800 ng of DNA was sonicated to an average of 300 bp fragments length, and was used for subsequent library preparation that includes blunting of DNA, A-tailing, ligation of sequencing adapters, and amplification of library.

Total (ribodepleted) RNA-seq Library preparation. 10 million cells were sorted and then whole RNA was isolated using the RNeasy Plus Mini Kit (Qiagen). Ribo-zero RNA-seq libraries were prepared using the TruSeq Stranded Total RNA Library Prep Gold kit (Illumina) with minor modifications. The starting RNA was 800 ng. Ribosomal RNAs were depleted using magnetic beads. Next, RNA was fragmented, and cDNA was synthesized using Superscript II (Invitrogen). After A-tailing and adaptor ligation, libraries were generated by amplifying the cDNA for 12 cycles.

Statistical Analysis. For mouse experiments, Mantel-Cox test and Gehan-Brenslow-Wilcoxon test were applied as indicated and the p values are shown for each figure. Statistical evaluations were performed using the unpaired t test. Data are mean±SEM. Asterisks indicate statistically significant differences: ****P<0.0001, ***P<0.001, **P<0.01, *P<0.05. If not otherwise indicated the p value was not statistically significant (p>0.05). In the graphs each dot represents a mouse. For all the experiments the inventors used sufficient number of mice to ensure adequate power for these conclusions. Mice from different litters and of different sex were evaluated. In addition, the inventors ensured that a minimum of 2 independent experiments was performed in each case. For the two-sample Kolmogorov-Smirnov test related to methylation analysis, the D statistic and pvalues were calculated using the ks.test function as implemented in R. In all tests, the alternative hypothesis is that CDF of WT lies below that of TET.

Example 20: DNA methylation and heterochromatin dysfunction in senescence and aging DNA cytosine methylation (hereafter, DNA methylation) is a classic “epigenetic” mark. It is controlled by the functional interplay between two families of enzymes: DNA methyltransferases (DNMTs) and TET methyl-cytosine dioxygenases, which control DNA methylation and demethylation respectively. The “maintenance” methyltransferase DNMT1, and the “de novo” methyltransferases DNMT3A and DNMT3B, transfer a methyl group from S-adenosyl-methionine (SAM) to the 5 position of cytosine to generate the “fifth base” 5-methylcytosine (5mC). The 3 mammalian TET proteins (TET1, TET2, TET3) are Fe(II) and alpha-ketoglutarate (αKG)-dependent dioxygenases that oxidize the methyl group of 5-methylcytosine (5mC) to 5-hydroxymethyl-cytosine (5hmC), 5-formylcytosine (5fC) and 5-carboxylcytosine (5caC) in DNA. TET proteins and oxi-mC bases are essential for all the dynamic DNA methylation that occurs in mammalian genomes—whether during embryogenesis, cell lineage specification in developing organs, or cell differentiation in response to environmental cues.

DNA methylation is markedly aberrant in aged and senescent cells. Focal increases of DNA methylation, primarily in euchromatic regions, occur concomitantly with widespread losses of DNA methylation, predominantly in heterochromatic regions. The mechanisms underlying these genome-wide changes in DNA methylation patterns are not known, but their consequences for the cellular function of aged cells are likely to be severe as described below.

DNA methylation is also aberrant in cancer, notably, the patterns of dysregulated DNA methylation—focal DNA hypermethylation in euchromatin and widespread DNA hypomethylation in heterochromatin—are similar in malignant and aged cells. In cancer, heterochromatic DNA hypomethylation is associated with “hetero-chromatin dysfunction”, whose characteristics include (i) reactivation of transposable elements (TEs), (ii) increased levels of unusual DNA structures (e.g., G-quadruplexes (G-quads) and R-loops); and (iii) increased levels of genomic aberrations, including single nucleotide and copy number variations (SNVs, CNVs), chromosomal translocations, centromere instability and aneuploidies. The stochastic mutations that drive cancer progression are facilitated by dysregulated expression of p53, DNA repair enzymes and various proteins involved in DNA damage repair. Cells that express the mutated genes are subject to selection, promoting the clonal expansion and clonal evolution characteristic of cancers. TE reactivation is also associated with increased inflammation, a common feature of aging, senescence and cancer.

As shown hereinabove, profound TET deficiency leads to aggressive cancers. Notably, cells with loss-of-function mutations in one or more TET genes have DNA methylation patterns resembling those of malignant, old or senescent cells; they also show many features of heterochromatin dysfunction, including TE reactivation, increased G-quads and R-loops, increased DNA damage, and multiple genome abnormalities. These points are highly relevant for cancer in general: not only are TET2 mutations frequent in human blood cancers, but many solid as well as hematological malignancies harbor mutations in genes encoding metabolic enzymes that directly or indirectly regulate TET function.

Like cancer cells, aging cells accumulate mutations with time, and many of these are in cancer driver genes. However, whether aged cells show other genomic features of heterochromatin dysfunction is not known. Here we will draw on our background in cancers with TET/DNMT deficiencies, to conduct a comprehensive analysis of alterations in DNA methylation and their relation to cellular function during aging. The present inventors can use the present invention to detect the genomic features of heterochromatin dysfunction in old/senescent cells. The present invention can be used to determine senescence and aging associated metabolic alterations known to decrease TET activity. The present invention can be used to reverse or delay the onset of senescence by preventing DNA hypomethylation in aging cells. Finally, the present invention can be used to induce senescence and heterochromatin dysfunction by targeting TET proteins.

These results represent the first systematic comparison of the regulation of DNA methylation in aged and malignant cells, and its relation to alterations in the metabolic and genomic features of these cells. (i) the present invention can be used to determine how DNA cytosine methylation is regulated during physiological aging versus oncogenesis, and identify features that are unique or common to these processes; (ii) illuminate the mechanistically elusive relation between aging and oncogenesis, and inform or modify strategies for cancer therapy or for slowing the rate of cellular aging; (iii) show the relation of DNA methylation to heterochromatin function and integrity and the understanding of heterochromatin, a genomic compartment that is currently only imperfectly understood.

As shown above, DNA cytosine methylation shows similar dysregulation in cancers in general, in cancers with TET/DMNT deficiency, and in aged/senescent cells. In all three cases, the cells display increased DNA methylation in euchromatic regions, but also contain broad “partially methylated domains” (PMDs) that show decreased DNA methylation as depicted in FIGS. 34 and 18. These widespread regions of decreased DNA methylation occur predominantly in heterochromatic regions, defined by late replication during S phase, association with the nuclear lamina, histone 3 lysine 9 (H3K9) methylation, and a paucity of CG dinucleotides that are the predominant sites of DNA methylation in the genome. This pattern of aberrant DNA methylation, defined by focal increases of DNA methylation in euchromatin and widespread losses of DNA methylation in heterochromatin, is well established in cancer, and there is emerging recognition that it also occurs in old/senescent cells (FIG. 34).

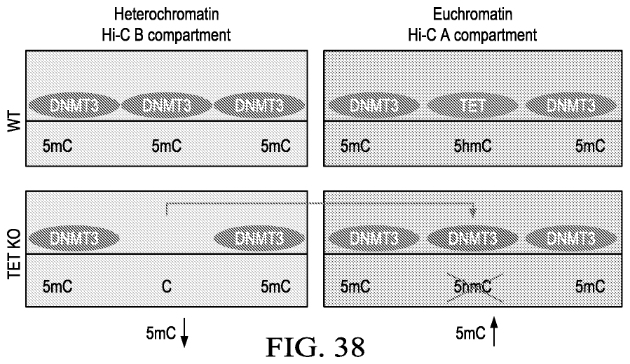

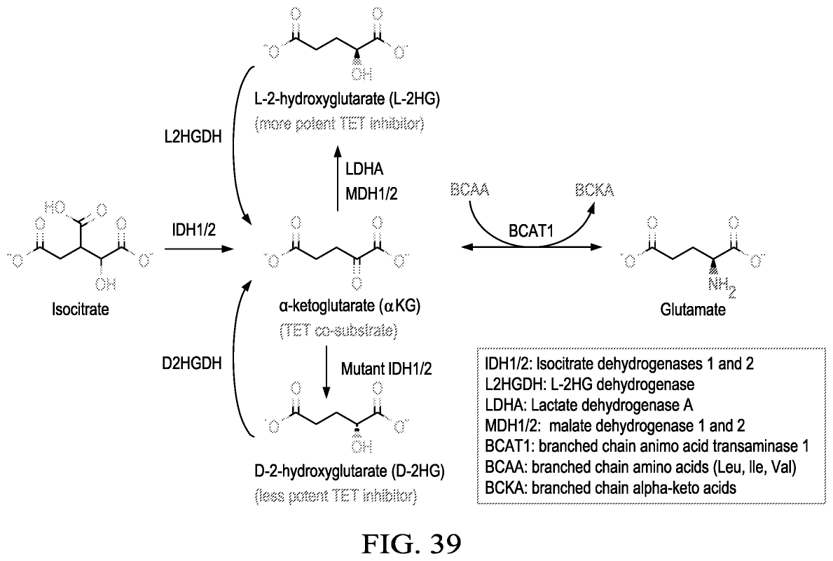

Surprisingly, the DNA methylation patterns of TET-deficient genomes closely resemble those of aged and cancer genomes (FIG. 38). The present inventors examined mice with Tet2 and Tet3 deficiency induced in developing T cells with CD4Cre (Tet2/3 DKO mice and T cells). These mice display an aggressive T cell malignancy with dramatic antigen-driven expansion and oncogenic transformation of “NKT” cells, a normally minor subpopulation of T cells that develop in the thymus and are self-reactive. The uncontrolled NKT cell expansion in Tet2fl/fl Tet3 fl/fl CD4Cre mice is antigen- and signal-dependent and develops into an oligoclonal NKT cell malignancy in all Tet2/3 DKO mice by 5 weeks of age. “Young” NKT cells from 4-week-old Tet2/3 DKO mice (a week before the appearance of perceptible disease) can transfer the malignancy to fully immunocompetent recipient mice. The present inventors performed Hi-C and whole-genome bisulphite sequencing (WGBS) on purified NKT cells isolated from wildtype (WT) mice, young Tet2/3 DKO mice, and the secondary recipients of “transferred and expanded” (exp) Tet2/3 DKO NKT cells, partitioned their genomes into euchromatic (Hi-C A) and hetero-chromatic (Hi-C B) compartments, and examined their DNA methylation patterns at megabase-scale resolution (FIG. 38). Thus, like the genomes of old and cancer cells generally, the DNA methylation patterns of TET-deficient genomes show focal hypermethylation in euchromatin and widespread losses of DNA methylation in heterochromatin.

While the focal hypermethylation in euchromatin in the genomes of TET-deficient cells was unsurprising, the wide-spread heterochromatic hypomethylation was unexpected. Biochemically, TET enzymes mediate the loss of DNA methylation (FIG. 14), thus it was generally assumed that TET deficiency would result in increased DNA methylation, and that this in turn would result in oncogenesis by promoting the silencing of tumor suppressor genes. In fact, the present inventors observed increased DNA methylation in the genomes of TET-deficient cells, but this hypermethylation is confined to the euchromatic Hi-C A compartment (FIG. 18, tracks 7, 8), since 5hmC is also present primarily in euchromatic, gene-rich areas of the genome.

Nevertheless, the analyses of the DNA methylation status of tumors that either developed naturally in humans or arose in the mouse models of TET deficiency, showed clearly that TET deficiency was also associated with widespread losses of DNA methylation in heterochromatic regions of the genome (FIG. 18). In addition, the inventors re-analyzed published data from other laboratories on various types of TET-deficient cells—mouse embryonic stem cells (mESC), neural precursor cells (NPC) differentiated from Tet2-deficient mouse ESC, and mouse hematopoietic stem/precursor cells (HSPC)—the inventors found that in every case, TET-deficient cells showed increased DNA methylation in the euchromatic Hi-C A compartment, occurring concomitantly with loss of DNA methylation in the heterochromatic Hi-C B compartment. The phenomenon of widespread DNA hypomethylation in TET-deficient cells was also noted by other, however, they failed to follow-up because the hypomethylated regions did not correspond to annotated DNA elements. Cells from humans with germline TET2 mutations also exhibit decreased methylation in the heterochromatic compartment, in this case defined by H3K9me3.

The present invention can be used to determine the relation between TET and DNMT loss-of-function, the consequent progressive loss of DNA methylation in heterochromatin, and the development of genomic abnormalities due to heterochromatin dysfunction. The present invention can be used to study old and senescent cells and compare those data with those from malignant cells, to determine whether the similar patterns of dysregulated DNA methylation observed in old cells versus cancers due to TET or DNMT dysfunction arise from similar metabolic aberrations and lead to overlapping genomic consequences in these two scenarios. As shown hereinabove, an important point is that TET loss-of-function can occur independently of TET coding-region mutations, as a consequence of metabolic aberrations or mutations in metabolic enzymes that interfere with TET enzymatic activity.

The present invention can be used to link DNA methylation changes to the genomic aberrations observed in both cancer and aging. Specifically, the present invention can be used to determine whether in cancer or in aging, loss of DNA methylation in heterochromatin arising from DNMT or TET deficiency (including mutations in metabolic enzymes as described below) can lead to heterochromatin dysfunction, promoting genomic aberrations including reactivation of transposable elements (TEs), increased R-loops and G-quads, chromosomal translocations, CNVs, SNVs and indels, as well as centromere instability and aneuploidies. These processes will in turn promote clonal selection, acquisition of mutations and genomic abnormalities, and possibly clonal evolution in both aging and cancer.

Cancerous and aged cells acquire similarly dysregulated DNA patterns of DNA methylation and share features of heterochromatin dysfunction. The present invention can be used for a systematic comparison of these two processes using human and mouse cells from young and old individuals, as well as information derived from human cancers and mouse cancer models. DNA hypomethylation in heterochromatin may lead to different types of consequences in cancer and aging.

The present invention can also be used for the systematic analysis of the regulation of DNA methylation—specifically heterochromatic DNA hypomethylation—in aged and senescent cells. The present invention can be used to determine the manner in which an altered pattern of DNA methylation, relates either causally or indirectly, to the altered metabolic and genomic features of these cells. The similarities and differences between regulation of DNA cytosine methylation during physiological aging versus oncogenesis are identified, as well as features that are unique or common to these two processes. In addition to illuminating the mechanistic relation between heterochromatic DNA methylation, aging and cancer, the invention can be used to understand heterochromatin, a genomic compartment that is currently very poorly understood. Thus, the present invention can be used both for cancer therapy and for slowing the rate of cellular aging.

DNMT and TET enzymes. DNA cytosine methylation is controlled by the functional interplay between DNA methyltransferases (DNMTs) and TET methylcytosine dioxygenases, enzyme families that control DNA methylation and demethylation respectively. In most cells, the vast majority (95-99%) of all DNA methylation occurs symmetrically at CpG (CG) sequences. The cycle of DNMT-mediated cytosine methylation and TET-mediated DNA demethylation is shown in FIG. 14. (a) DNMTs. There are three DNMTs and three TET enzymes in mammals. Although it has a modicum of de novo activity, DNMT1 is primarily a “maintenance” methyltransferase that—with its partner UHRF1—restores symmetrical methylation at “hemi-methylated” CpG sequences that arise after DNA replication; whereas DNMT3A and DNMT3B are “de novo” methyltransferases that can transfer a methyl group to fully unmodified cytosines in the CpG context. (b) TET dioxygenases successively oxidise the methyl group of 5mC to 5-hydroxymethylcyto-sine (5hmC), 5-formylcytosine (SIC) and 5-carboxylcytosine (5caC)(FIG. 14). Tet1 and Tet2 are expressed in mouse embryonic stem cells (mESC), while Tet3 is expressed in the oocyte and in the early zygote, declines by the blastocyst stage, and is re-expressed with Tet2 in most differentiated somatic cells. TET enzymes are present at promoters and enhancers; the 5hmC and oxi-mC bases that they generate are primarily present in euchromatin and are most enriched in gene bodies of the most actively transcribed genes and at the most active enhancers. Tet2 and Tet3 function redundantly in cells of the immune and hematopoietic systems: mouse T, B and myeloid cells that have been deleted for both the Tet2 and Tet3 genes show substantially greater functional impairment and/or propensity to oncogenesis compared to cells deleted for either Tet2 and Tet3 alone. Similarly, double deletion of Tet2 and Dnmt3A in mouse hematopoietic stem/precursor cells leads to far greater functional impairment of hematopoiesis, and far more rapid development of myeloid leukemia, compared to cells deficient for either Tet2 or Dnmt3a alone.

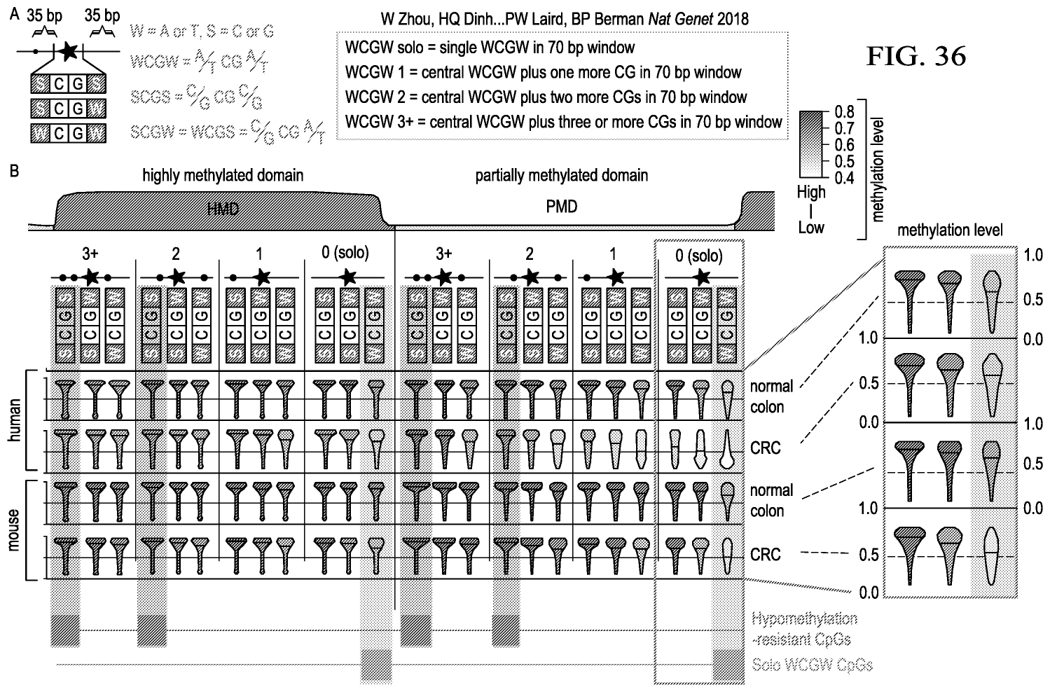

Mechanisms of DNA demethylation. (a) “Passive” (replication-dependent) DNA demethylation. During DNA replication, the 5-methylcytosine (5mC) complementary to the G in the template strand is replaced with unmodified cytosine (C) in the newly synthesized DNA strand. The resulting hemimethylated CpG sequences are rapidly remethylated by the maintenance methyl-transferase complex of DNMT1 with UHRF1. UHRF1 recognizes hemimethylated CpGs through its SRA domain, and the DNMT1/UHRF1 complex travels with the DNA replication complex through its interaction with proliferating cell nuclear antigen (PCNA). This process restores symmetrical DNA methylation to newly synthesized DNA and is responsible for the well-known heritability of DNA methylation (FIG. 36). However, hemi-modified oxi-mCs are not recognized by the UHFR1 SRA domain, and hence methyl-CpGs in which one or both 5mC's have been oxidised by TET protein progressively lose methylation with each cycle of DNA replication. (b) TDG-dependent “active” DNA demethylation. The DNA repair enzyme thymine DNA glycosylase (TDG), which typically excises thymine from T:G mis-matches in DNA, has a second function in which it can excise 5fC and 5caC from normal 5fC:G and 5caC:G basepairs. If the resulting abasic site is repaired through base excision repair, the original 5fC and 5caC are replaced by unmodified C in a manner that does not depend on DNA replication. The present inventors found, however, that TDG-deficient cells show no impairment in T helper 2 (Th2) and induced T regulatory (iTreg) cell differentiation, both processes that involve many cycles of cell proliferation. Rather, increased production of the cytokine IL-4 by Th2 cells, and increased expression of the transcription factor Foxp3 by iTreg cells, can be completely accounted for by passive replication-dependent loss of DNA modifications in the proliferating and differentiating cells. A small amount of 5fC and 5caC accumulates in the absence of TDG and are most likely lost through passive replication-dependent dilution in these differentiating T cells; thus TDG-dependent active DNA demethylation accounts for only a very small fraction of DNA demethylation during the processes of Th2 and iTreg cell differentiation. Even in bone marrow-derived macrophages, which stop proliferating and differentiate into cytokine-producing cells upon stimulation with LPS and IL-4, TDG deficiency has almost no effect on cytokine production and differentiation.

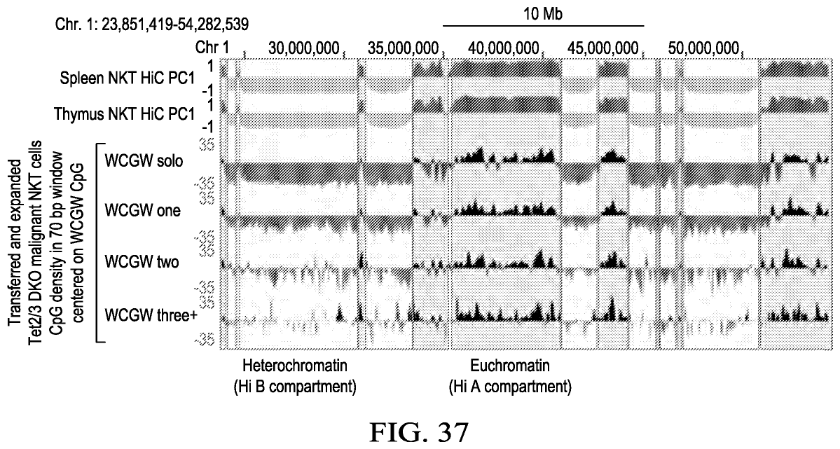

Preferential loss of DNA methylation at solo-CpGs in heterochromatin. As shown in FIG. 34, CD4 T cells from an old (103-year-old) individual displayed a clear difference in their genome-wide patterns of DNA methylation compared to CD4 T cells from a newborn human; the large heterochromatic PMDs in the old cells showed the same general distribution as those observed in a T cell leukemia. The loss of methylation observed in both old and malignant cells, including the TET-deficient cancers, occurs predominantly at widely dispersed CpGs (“solo-CpGs”) in the WCGW or ^{A}/_{T}CG^{A}/_{T} sequence context (W is A or T)(FIGS. 37, 38). WCGW solo-CpGs are more frequent in heterochromatin, which is AT-rich, than in CG-rich euchromatin. The preferential loss of methylation at WCGW solo-CpGs is because the DNMT1/UHRF1 maintenance DNA methyltransferase complex recognizes CpGs in a WGGW sequence only poorly, and DNMT1 is processive and more efficient at restoring methylation to closely spaced hemi-methylated CpGs. Moreover, since heterochromatin replicates late during S phase whereas euchromatin replicates early, a likely explanation (but not a limitation of the present invention) for the existence of partially methylated domains is that DNA methylation is lost preferentially in heterochromatin as a function of the number of cell divisions that a cell has undergone, because the DNMT1/UHRF1 complex fails to “keep up” with replication to complete DNA methylation in heterochromatin in rapidly-replicating cells. This scenario is plausible for (i) old/senescent cells, which through their lifetime have undergone a larger number of cell divisions compared to young cells, and (ii) cancer cells, which by definition replicate faster than normal cells, as shown hereinabove for the Tet2/3-deficient NKT cell malignancy introduced in FIG. 34, which also shows the most striking decrease in DNA methylation at solo WCGW CpGs (FIG. 37).

By way of explanation, and in no way a limitation of the present invention, there are several additional mechanisms, not mutually exclusive, to explain the loss of heterochromatic DNA methylation in old/senescent and TET-deficient cells. For instance, the inventors have shown that the deubiquitinase USP7 and all three DNMTs are downregulated at the protein level as IMR90 human fibroblasts approach senescence; moreover, CRISPR-mediated knockdown or small molecule-mediated inhibition of USP7 in young IMR90 fibroblasts leads to loss of DNMTs and decreased global levels of 5mC. Through analysis of published data in mouse ES cells, the inventors have also shown a functional interplay between DNMTs and TETs: TET1 occupied primarily euchromatic regions in WT mESC, but in the absence of TET1, DNMT3A showed a striking relocalization to the regions previously occupied by TET1 in the parental WT ESC (results summarized in FIG. 37). This finding implies a common scaffold structure in euchromatin that binds both TETs and DNMTs, but with higher affinity for TETs, and could also explain some of the euchromatic focal hypermethylation and heterochromatic loss of DNA methylation in mouse ESC. Thus, the present invention can be used to examine the role of USP7 in regulating TET and DNMT activity in old and senescent cells, and also map the genome-wide distribution of TETs and DNMTs in these cells.

Metabolic enzymes that modulate TET catalytic activity. Loss-of-function mutations in the TET2 and DNMT3A genes are frequent in human hematological malignancies, and coding region mutations in TET genes have been associated with the pathogenesis of certain solid cancers including endometrial cancers, colorectal cancer, and melanoma. Overall, however, coding region mutations in TET and DNMT genes are relatively uncommon in solid cancers. However, many cancers (both hematological and solid cancers) display a profound functional loss of TET activity, despite the absence of coding region mutations in TET or DNMT genes, either because of metabolic perturbations such as hypoxia, or because of aberrant expression or recurrent mutations in metabolic enzymes. Among such recurrent mutation are recurrent dominant mutations in the isocitrate dehydrogenase enzymes IDH1 and IDH2 mutations in acute myeloid leukemia (AML) and glioblastoma multiforme (GBM) which result in aberrant generation of the “oncometabolite” 2-hydroxyglutarate, a competitive inhibitor of αKG-dependent dioxygenases, by the mutant enzymes; succinate dehydrogenase mutations in breast cancer, which result in increased levels of succinate, a product and feedback inhibitor of TET enzymatic activity; L-2-hydroxyglutarate dehydrogenase mutations in diffuse large B cell lymphoma (DLBCL) and renal cell carcinomas (see Lio et al., J BioSci 2020; Ko et al, Curr Opin Cell Biol 2015; Huang et al., Trends Genetics 2014; and references therein). Together, these observations emphasize that loss-of TET activity is a common feature of many cancers. The present inventors can artificially induce TET deficiency during aging and senescence by genetic manipulation of these enzymes.

The present invention can be used to determine the inflammatory and interferon response signatures observed both in cancers and in aged/senescent cells stem from the genomic features of heterochromatin dysfunction, particularly the reactivation and perhaps even the frank transposition of transposable elements (TEs) located in heterochromatin (FIG. 34). TEs that are located primarily in heterochromatin include long interspersed nuclear elements (LINES) and many families of endogenous retroviruses (ERVs), whose expression is in many cases controlled by DNA methylation. The present invention can be used to determine whether TE reactivation and the consequent increased levels of RNA from the TEs are associated with (i) increased expression of interferon-induced genes, (ii) the senescence-associated secretory phenotype (SASP), (iii) increased levels of R-loops and G-quads and (iv) increased propensity to mutations. The present invention can be used to test for metabolic aberrations which lead to decreased TET function, and are already associated with altered DNA methylation patterns in human cancers, also provoke the alterations in DNA methylation observed in aging cells. The present invention can also be used to test whether increased expression mediated by CRISPRa, culture with Vitamin C, and expression of heterochromatin-targeted versions of DNMTs, increase intra-cellular TET and DNMT activity. As shown hereinabove, the inventors tested heterochromatin-targeted DNMT3A in TET-deficient cancers. Thus, the present invention can be used to treat the cells to delay, prevent or even reverse the onset of senescence by restoring DNA methylation in heterochromatin. The present invention can be used to induce senescence by genetic manipulation of the levels of TETs, DNMTs or the deubiquitinase Ubiquitin Specific Peptidase 7 (USP7), or targeting TET proteins to heterochromatin, thereby provoking DNA demethylation in heterochromatin and therefore heterochromatin dysfunction.

Detecting the genomic features of heterochromatin dysfunction in old/senescent cells. The present invention can be used to assess the relation of heterochromatic DNA hypomethylation (determined by whole-genome bisulphite sequencing, WGBS) to clonal expansion and TE reactivation and transposition in young and senescent cells. The present invention can be used to assess the relation of TE reactivation and transposition to the senescence-associated secretory phenotype (SASP) and inflammatory responses (expression of interferon-induced genes) in young and senescent cells. The present invention can be used to assess the levels of R-loops and G-quads, as well as DNA damage, by flow cytometry in bulk populations of young and senescent cells. We will also measure the distribution of R-loops and G-quads in these populations by MAPR or immunoprecipitation with a catalytically dead RNAse H1 and by DNA immunoprecipitation with the G-quad-recognizing antibody BG4, respectively, and relate the distribution and magnitude of these non-B-form DNA structures to the levels of expression of the associated genes or transposable elements. In clones expanded from single cells—either young or approaching senescence—whole-exome and whole-genome sequencing can be used to assess mutations, CNVs, SNVs, indels, translocations, and aneuploidies. These studies can also test lymphoid (T cells, B cells) and myeloid cells isolated from young and old mice.

The present invention can be used to show TE reactivation in T cell leukemia model, myeloid leukemia model, show increased R-loops and G-quads in B cell lymphoma model. show chromosomal translocations by HiC, and show compartment identification by HiC, and WGBS for hypomethylation in chromatin. Barcoding of myeloid cells can be used to show increased clonality and show centromere instability and aneuploidies. The experiments can be repeated with young and old mouse cells— T cells, B cells, myeloid cells, IMR90 fibroblasts, proliferating and senescent and human young and old cells, Barcode IMR90 cells, and WES of clones to find mutations. In some cases, WGS of clones can be used to check mutations in noncoding genome regions as well.

The present invention can be used to examine the metabolic, genomic and (in some cases) proteomic features of human lymphoid and myeloid cells isolated from the peripheral blood of individuals of different ages, as well as the corresponding cell types from spleen and lymph nodes of young and old mice. DNA methylation is regulated by DNA methyltransferases (DNMTs) which generate 5-methylcytosine (5mC) in DNA, and by TET methylcytosine dioxygenases which generate oxidized methylcytosines that are intermediates in DNA demethylation. Mutations or metabolic aberrations that lead to DNMT or TET loss-of-function result in haematopoietic malignancies. This example provides a comprehensive view of the relation of DNA methylation changes to aging and oncogenesis.

ChIP-seq for TETs and DNMTs. Determining senescence and aging associated with metabolic alterations known to decrease TET. The present invention can be used to observe heterochromatic DNA hypomethylation in old/senescent cells reflects a decrease in the function of TET and/or DNMT. The present invention can be used to measure the levels of αKG, 2HG (both L- and D-stereoisomers), succinate and fumarate in young and senescent cells, as well as mRNA and protein expression of enzymes that modulate the levels of these metabolites. Decreased αKG, increased D- or L-2HG, and increased levels of succinate or fumarate are known to result in decreased TET activity. The present invention can be used to relate these metabolic changes to levels of TET activity measured by flow cytometry for 5hmC. The present invention can be used to observe metabolic aberrations known to be associated with decreased TET activity. The present invention can also observe losses of DNA methylation in heterochromatin and the genomic features of heterochromatin dysfunction, e.g., in B, T and myeloid cells isolated from young and old mice.

Identify the metabolic alterations that affect heterochromatin integrity in old/senescent cells. Briefly, these metabolic aberrations include: (i) increased intracellular levels of the D stereoisomer of 2-hydroxyglutarate (D-2HG), a competitive inhibitor of alpha-ketoglutarate (αKG), that is caused by dominant recurrent mutations in the isocitrate dehydrogenases IDH1 and IDH2 in glioblastoma and acute myeloid leukemia; (ii) increased intracellular levels of the more potent inhibitor, the L stereoisomer of 2-HG 2HG (L-2HG), caused by loss-of-function mutations in the enzyme L-2HG dehydrogenase (L2HGDH), primarily in renal cancers; (iii) increased intracellular levels of succinate caused by loss-of-function mutations of the enzyme succinate dehydrogenase (SDH) in cancers and in the more potent L-2HG caused by 1 succinate (FIG. 34), and hypoxia. The present inventors can measure αKG and 2-HG in young and old mouse cells—T cells, B cells, myeloid cells and also IMR90 fibroblasts, proliferating and senescent and human young and old cells.

The present invention can be used to reverse or delay the onset of senescence by preventing DNA hypomethylation in aging cells. The present inventors have shown that introducing a version of DNMT3A that is targeted specifically to heterochromatin by fusion with HP1b into precancerous cells can delay cancer progression. The present invention can be used to introduce either an inducible (degron-tagged) full-length DNMT3A or the HP1β-DNMT3A fusion protein into young cells and ask if overexpression of full-length or heterochromatin-targeted DNMT delays the onset of senescence, increases DNA methylation status in heterochromatin, and ameliorates the features of heterochromatin dysfunction described above. The present inventors can use Vitamin C, a well-established activator of TET proteins and other αKG-dependent dioxygenases, to ask whether increasing TET activity has similar effects (delaying senescence, preventing DNA hypomethylation in heterochromatin, and countering heterochromatin dysfunction). The present invention can also be used to test whether CRISPRa-mediated activation of TETs and DNMTs in proliferating cells delays the onset of senescence, which can also be tested on lymphoid and myeloid cells isolated from young and old mice.

The present invention can also be used to reverse or delay the onset of senescence by preventing heterochromatic DNA hypomethylation in aging cells. It was found that loss of DNMTs in IMR90 fibroblasts, which points to USP7 going down. The present invention can be used to restore USP7 or DNMTs ectopically (dTag versions).

For example, the present invention can be used to induce senescence and heterochromatin dysfunction by targeting TET proteins. The present invention found that senescent cells show a striking decrease in the levels of all three DNMT proteins, as well as in the levels of USP7, a deubiquitinase whose loss or mutation is thought to result in increased degradation rates of a large number of chromatin-associated proteins, including p53, MDM2 and DNMT3A. Moreover, a USP7 inhibitor, when used at micromolar concentrations, downregulates DNMT levels and decreases the global levels of DNA methylation. The present invention can be used to explore the relation between USP7 expression and activity, the levels of DNMT proteins, and genome-wide DNA methylation status in exponentially growing and senescing cells, and ask if downregulation of USP7 results in decreased DNMT levels and global loss of DNA methylation. The present invention can be used to downregulate DNMT protein levels by CRISPR-mediated knockdown of either DNMT or (if warranted) USP7, and ask whether these manipulations accelerate the onset of senescence and result in SASP, increased expression of interferon-induced genes, and the genomic features of heterochromatin dysfunction above. The present invention can be used to determine whether the catalytic activities of TETs and DNMTs are required to accelerate senescence and confer the other features of heterochromatin dysfunction, by culturing with cell-permeant L-2HG and decitabine respectively. The present invention can be used to target TET proteins to heterochromatin by fusion with HP1b, as described for DNMT3A above, and ask if this results in DNA demethylation in heterochromatin and consequent heterochromatin dysfunction, which can also be conducted with lymphoid and myeloid cells isolated from young and old mice. The present invention can be used to determine senescence and heterochromatin dysfunction by targeting TET proteins to heterochromatin. The present invention also reverse the strategy and put TETs into heterochromatic regions to determine whether they can be demethylated and whether there will be DNA demethylation in heterochromatin and consequent heterochromatin dysfunction.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. M. Tahiliani et al., Conversion of 5-methylcytosine to 5-hydroxymethylcytosine in mammalian DNA by MLL partner TET1. Science 324, 930-935 (2009).
2. S. Ito et al., Tet proteins can convert 5-methylcytosine to 5-formylcytosine and 5-carboxylcytosine. Science 333, 1300-1303 (2011).
3. Y. F. He et al., Tet-mediated formation of 5-carboxylcytosine and its excision by TDG in mammalian DNA. Science 333, 1303-1307 (2011).
4. X. Wu, Y. Zhang, TET-mediated active DNA demethylation: mechanism, function and beyond. Nat Rev Genet 18, 517-534 (2017).
5. M. Ko et al., Impaired hydroxylation of 5-methylcytosine in myeloid cancers with mutant TET2. Nature 468, 839-843 (2010).
6. F. Delhommeau et al., Mutation in TET2 in myeloid cancers. N Engl J Med 360, 2289-2301 (2009).
7. S. M. Langemeijer et al., Acquired mutations in TET2 are common in myelodysplastic syndromes. Nat Genet 41, 838-842 (2009).
8. F. Lemonnier et al., Recurrent TET2 mutations in peripheral T-cell lymphomas correlate with TFH-like features and adverse clinical parameters. Blood 120, 1466-1469 (2012).
9. L. Cimmino et al., TET1 is a tumor suppressor of hematopoietic malignancy. Nat Immunol 16, 653-662 (2015).
10. M. Ko, J. An, A. Rao, DNA methylation and hydroxymethylation in hematologic differentiation and transformation. Curr Opin Cell Biol 37, 91-101 (2015).
11. S. Raffel et al., BCAT1 restricts αKG levels in AML stem cells leading to IDHmut-like DNA hypermethylation. Nature 551, 384-388 (2017).
12. G. C. Hon et al., 5mC oxidation by Tet2 modulates enhancer activity and timing of transcriptome reprogramming during differentiation. Mol Cell 56, 286-297 (2014).
13. W. A. Flavahan et al., Insulator dysfunction and oncogene activation in IDH mutant gliomas. Nature 529, 110-114 (2016).
14. A. Tsagaratou et al., TET proteins regulate the lineage specification and TCR-mediated expansion of iNKT cells. Nat Immunol 18, 45-53 (2017).
15. F. Lu, Y. Liu, L. Jiang, S. Yamaguchi, Y. Zhang, Role of Tet proteins in enhancer activity and telomere elongation. Genes Dev 28, 2103-2119 (2014).
16. J. An et al., Acute loss of TET function results in aggressive myeloid cancer in mice. Nat Commun 6, 10071 (2015).
17. E. Lieberman-Aiden et al., Comprehensive mapping of long-range interactions reveals folding principles of the human genome. Science 326, 289-293 (2009).
18. B. van Steensel, A. S. Belmont, Lamina-Associated Domains: Links with Chromosome

Architecture, Heterochromatin, and Gene Repression. Cell 169, 780-791 (2017).
19. I. Hiratani et al., Global reorganization of replication domains during embryonic stem cell differentiation. PLoS Biol 6, e245 (2008).
20. B. P. Berman et al., Regions of focal DNA hypermethylation and long-range hypomethylation in colorectal cancer coincide with nuclear lamina-associated domains. Nat Genet 44, 40-46 (2011).
21. G. C. Hon et al., Global DNA hypomethylation coupled to repressive chromatin domain formation and gene silencing in breast cancer. Genome Res 22, 246-258 (2012).
22. W. Zhou et al., DNA methylation loss in late-replicating domains is linked to mitotic cell division. Nat Genet 50, 591-602 (2018).
23. S. B. Baylin, P. A. Jones, Epigenetic Determinants of Cancer. Cold Spring Harb Perspect Biol 8 (2016).
24. B. Schuster-Böckler, B. Lehner, Chromatin organization is a major influence on regional mutation rates in human cancer cells. Nature 488, 504-507 (2012).
25. Y. Yin et al., Impact of cytosine methylation on DNA binding specificities of human transcription factors. Science 356 (2017).
26. C. W. Lio et al., Tet2 and Tet3 cooperate with B-lineage transcription factors to regulate DNA modification and chromatin accessibility. Elife 5 (2016).
27. X. Zhang et al., DNMT3A and TET2 compete and cooperate to repress lineage-specific transcription factors in hematopoietic stem cells. Nat Genet 48, 1014-1023 (2016).
28. Y. Huang et al., The behaviour of 5-hydroxymethylcytosine in bisulfite sequencing. PLoS One 5, e8888 (2010).
29. B. Bonev et al., Multiscale 3D Genome Rewiring during Mouse Neural Development.

Cell 171, 557-572.e524 (2017).
30. A. Poleshko et al., Genome-Nuclear Lamina Interactions Regulate Cardiac Stem Cell Lineage Restriction. Cell 171, 573-587.e514 (2017).
31. Y. Huang et al., Distinct roles of the methylcytosine oxidases Tet1 and Tet2 in mouse embryonic stem cells. Proc Natl Acad Sci USA 111, 1361-1366 (2014).
32. Y. C. Lin et al., Global changes in the nuclear positioning of genes and intra- and interdomain genomic interactions that orchestrate B cell fate. Nat Immunol 13, 1196-1204 (2012).
33. A. Bendelac, P. B. Savage, L. Teyton, The biology of NKT cells. Annu Rev Immunol 25, 297-336 (2007).
34. R. Z. Chen, U. Pettersson, C. Beard, L. Jackson-Grusby, R. Jaenisch, DNA hypomethylation leads to elevated mutation rates. Nature 395, 89-93 (1998).
35. A. Eden, F. Gaudet, A. Waghmare, R. Jaenisch, Chromosomal instability and tumors promoted by DNA hypomethylation. Science 300, 455 (2003).
36. F. Gaudet et al., Induction of tumors in mice by genomic hypomethylation. Science 300, 489-492 (2003).
37. L. B. Alexandrov et al., Signatures of mutational processes in human cancer. Nature 500, 415-421 (2013).
38. C. P. Walsh, J. R. Chaillet, T. H. Bestor, Transcription of IAP endogenous retroviruses is constrained by cytosine methylation. Nat Genet 20, 116-117 (1998).
39. P. Zeller et al., Histone H3K9 methylation is dispensable for Caenorhabditis elegans development but suppresses RNA:DNA hybrid-associated repeat instability. Nat Genet 48, 1385-1395 (2016).
40. Q. Zhu et al., Heterochromatin-Encoded Satellite RNAs Induce Breast Cancer. Mol Cell 70, 842-853.e847 (2018).
41. M. P. Crossley, M. Bocek, K. A. Cimprich, R-Loops as Cellular Regulators and Genomic Threats. Mol Cell 73, 398-411 (2019).
42. S. J. Boguslawski et al., Characterization of monoclonal antibody to DNA.RNA and its application to immunodetection of hybrids. J Immunol Methods 89, 123-130 (1986).
43. L. Couronné, C. Bastard, 0. A. Bernard, TET2 and DNMT3A mutations in human T-cell lymphoma. N Engl J Med 366, 95-96 (2012).
44. O. Odejide et al., A targeted mutational landscape of angioimmunoblastic T-cell lymphoma. Blood 123, 1293-1296 (2014).
45. G. Hu et al., Transformation of Accessible Chromatin and 3D Nucleome Underlies Lineage Commitment of Early T Cells. Immunity 48, 227-242.e228 (2018).
46. M. Jeong et al., Large conserved domains of low DNA methylation maintained by Dnmt3a. Nat Genet 46, 17-23 (2014).
47. R. Lister et al., Hotspots of aberrant epigenomic reprogramming in human induced pluripotent stem cells. Nature 471, 68-73 (2011).
48. X. Li et al., Tet proteins influence the balance between neuroectodermal and mesodermal fate choice by inhibiting Wnt signaling. Proc Natl Acad Sci USA 113, E8267-E8276 (2016).
49. H. A. Cruickshanks et al., Senescent cells harbour features of the cancer epigenome. Nat

Cell Biol 15, 1495-1506 (2013).
50. K. E. Bachman, M. R. Rountree, S. B. Baylin, Dnmt3a and Dnmt3b are transcriptional repressors that exhibit unique localization properties to heterochromatin. J Biol Chem 276, 32282-32287 (2001).
51. T. Baubec et al., Genomic profiling of DNA methyltransferases reveals a role for DNMT3B in genic methylation. Nature 520, 243-247 (2015).
52. M. Manzo et al., Isoform-specific localization of DNMT3A regulates DNA methylation fidelity at bivalent CpG islands. EMBO J 36, 3421-3434 (2017).
53. T. Gu et al., DNMT3A and TET1 cooperate to regulate promoter epigenetic landscapes in mouse embryonic stem cells. Genome Biol 19, 88 (2018).
54. K. Williams et al., TET1 and hydroxymethylcytosine in transcription and DNA methylation fidelity. Nature 473, 343-348 (2011).
55. S. Jaiswal et al., Age-related clonal hematopoiesis associated with adverse outcomes. N Engl J Med 371, 2488-2498 (2014).
56. L. M. Iyer et al., Lineage-specific expansions of TET/JBP genes and a new class of DNA transposons shape fungal genomic and epigenetic landscapes. Proc Natl Acad Sci USA 111, 1676-1683 (2014).
57. A. Janssen, S. U. Colmenares, G. H. Karpen, Heterochromatin: Guardian of the Genome. Annu Rev Cell Dev Biol 34, 265-288 (2018).
58. G. L. Xu et al., Chromosome instability and immunodeficiency syndrome caused by mutations in a DNA methyltransferase gene. Nature 402, 187-191 (1999).
59. B. Thienpont et al., Tumour hypoxia causes DNA hypermethylation by reducing TET activity. Nature 537, 63-68 (2016).
60. T. Mazor, A. Pankov, J. S. Song, J. F. Costello, Intratumoral Heterogeneity of the Epigenome. Cancer Cell 29, 440-451 (2016).

### REFERENCES FOR FIGS. 8-13

1. Y. Xi, W. Li, BSMAP: whole genome bisulfite sequence MAPping program. BMC Bioinformatics 10, 232 (2009).
2. X. Lin et al., BSeQC: quality control of bisulfite sequencing experiments. Bioinformatics 29, 3227-3229 (2013).
3. S. Heinz et al., Simple combinations of lineage-determining transcription factors prime cis-regulatory elements required for macrophage and B cell identities. Mol Cell 38, 576-589 (2010).
4. H. Li, R. Durbin, Fast and accurate long-read alignment with Burrows-Wheeler transform. Bioinformatics 26, 589-595 (2010).
5. M. Lienhard, C. Grimm, M. Morkel, R. Herwig, L. Chavez, MEDIPS: genome-wide differential coverage analysis of sequencing data derived from DNA enrichment experiments. Bioinformatics 30, 284-286 (2014).
6. I. Hiratani et al., Global reorganization of replication domains during embryonic stem cell differentiation. PLoS Biol 6, e245 (2008).
7. D. Peric-Hupkes et al., Molecular maps of the reorganization of genome-nuclear lamina interactions during differentiation. Mol Cell 38, 603-613 (2010).
8. A. Dobin et al., STAR: ultrafast universal RNA-seq aligner. Bioinformatics 29, 15-21 (2013).
9. Y. Jin, 0. H. Tam, E. Paniagua, M. Hammell, TEtranscripts: a package for including transposable elements in differential expression analysis of RNA-seq datasets. Bioinformatics 31, 3593-3599 (2015).
10. M. I. Love, W. Huber, S. Anders, Moderated estimation of fold change and dispersion for RNA-seq data with DESeq2. Genome Biol 15, 550 (2014).
11. A. McKenna et al., The Genome Analysis Toolkit: a MapReduce framework for analyzing next-generation DNA sequencing data. Genome Res 20, 1297-1303 (2010).
12. K. Cibulskis et al., Sensitive detection of somatic point mutations in impure and heterogeneous cancer samples. Nat Biotechnol 31, 213-219 (2013).
13. F. Blokzijl, R. Janssen, R. van Boxtel, E. Cuppen, MutationalPatterns: comprehensive genome-wide analysis of mutational processes. Genome Med 10, 33 (2018).
14. K. Wang, M. Li, H. Hakonarson, ANNOVAR: functional annotation of genetic variants from high-throughput sequencing data. Nucleic Acids Res 38, e164 (2010).
15. M. Ko et al., Ten-Eleven-Translocation 2 (TET2) negatively regulates homeostasis and differentiation of hematopoietic stem cells in mice. Proc Natl Acad Sci USA 108, 14566-14571 (2011).
16. M. Ko et al., TET proteins and 5-methylcytosine oxidation in hematological cancers. Immunol Rev 263, 6-21 (2015).
17. P. P. Lee et al., A critical role for Dnmt1 and DNA methylation in T cell development, function, and survival. Immunity 15, 763-774 (2001).
18. C. Plessy et al., clonotypeR-high throughput analysis of T cell antigen receptor sequences. bioRxiv .doi: 10.1101/028696 (2015).

What is claimed is:

1. A method of increasing, stimulating, inducing, promoting, enhancing or maintaining the genomic stability of a cell of a subject, the method comprising decreasing, reducing, inhibiting, suppressing, limiting or controlling loss of methylation of heterochromatin in the cell.

2. A method of modulating heterochromatin dysfunction in a cell of a subject, the method comprising activating, eliciting, stimulating, inducing, promoting, increasing or enhancing expression or activity in the cell of one or more DNA methyltransferase (DNMT) or one or more TET methyl-cytosine dioxygenases (TET) proteins, or both.

3. The method of claim 2, further comprising increasing the activity of, or overexpressing: one or more DNMTs, USP7, one or more TET methyl-cytosine dioxygenases (TET) proteins, increased expression of one or more DMNTs, USP7, or TETs by CRISPRa, Vitamin C, expression of one or more heterochromatin-targeted DNMTs, or expression of one or more heterochromatin-targeted.

4. The method of any one of claims 1 to 3, wherein the method comprises activating, eliciting, stimulating, inducing, promoting, increasing or enhancing expression or activity of: one or more DNA methyltransferase (DNMT) or one or more TET methyl-cytosine dioxygenases (TET) proteins, in the cell by administering to the subject an effective amount of an agent that increases the expression or activity of the one or more DNMTs or TETs.

5. The method of claim 4, wherein the method comprises restoring methylation, reducing defective chromosome segregation, reducing undesired cell proliferation, differentiation, or migration, or reducing heterochromatin aberrations, centromere aberrations, telomere aberrations, R-loops, G-quadruplexes, DNA damage, aneuploidies or cell defects or undesired cell proliferation, differentiation, or migration.

6. The method of any one of claims 1 to 4, wherein the method comprises decreasing, reducing, inhibiting, suppressing, limiting or controlling a heterochromatin dysfunction or genomic instability.

7. The method of claim 6, wherein the method comprises decreasing, reducing, inhibiting, suppressing, limiting or controlling an adverse symptom of the heterochromatin dysfunction or genomic instability in the subject.

8. The method of claim 7, wherein the adverse symptom of the heterochromatin dysfunction or genomic instability in the subject heterochromatin aberrations, centromere aberrations, telomere aberrations, R-loops, G-quadruplexes, DNA damage, aneuploidies or cell defects or undesired cell proliferation, differentiation, or migration.

9. The method of any one of claims 1 to 8, wherein the cell is at least one of: a cancer cell, a cell with one or more unstable chromosomes, an aged cell, or a senescent cell.

10. The method of any one of claims 1 to 9, wherein the method comprises decreasing, reducing, inhibiting, suppressing, limiting or controlling an adverse symptom of a neoplasia, neoplastic disorder, tumor, cancer or malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy.

11. The method of claim 10, wherein the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy.

12. The method of claim 6, wherein the heterochromatin dysfunction or genomic instability results in an undesirable or aberrant age-associated genome dysfunction, immune disorder or autoimmune response, disorder or disease.

13. The method of claim 12, wherein the method comprises decreasing, reducing, inhibiting, suppressing, limiting or controlling an adverse symptom of the undesirable or aberrant age-associated genome dysfunction, immune disorder, or autoimmune response, disorder or disease in the subject.

14. The method of any of claim 7, 8, 10, or 13, wherein the adverse symptom is chronic or acute.

15. The method of any one of claims 12 to 14, wherein the undesirable or aberrant age-associated genome dysfunction, immune disorder, or autoimmune response, disorder or disease, or symptom thereof, comprises hearing loss, presbycusis, increased cerumen production, loss of visual acuity, visual impairment, loss of vestibular function, sarcopenia, chronic inflammation, declining hormone levels, impaired muscle mitochondrial function, impaired muscle stem cell function, muscle weakness, immunosenescence, decrease in urologic function, cardiovascular disease, chronic ischemic heart disease, congestive heart failure, arrhythmia, atherosclerosis, peripheral vascular disease, hypertension, rheumatoid arthritis, juvenile rheumatoid arthritis, osteoarthritis, osteoporosis, short-term memory loss, dementia, Alzheimer's disease, progerias, Hutchinson-Gilford progeria syndrome (HGPS), Werner syndrome (WS), Cockayne syndrome (CS), Bloom syndrome (BS), ataxia-telangiectasia (A-T), xeroderma pigmentosum (XP), Rothmund-Thomson syndrome (RTS), centromere instability, telomere instability, facial anomalies syndrome (ICF), myelodysplasia syndrome (MDS), chronic lymphocytic leukemia (CLL), and acute myeloid leukemia (AML), psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, severe combined immunodeficiency (SCID), recombinase activating gene (RAG 1/2) deficiency, adenosine deaminase (ADA) deficiency, interleukin receptor common g chain (c) deficiency, Janus-associated kinase 3 (JAK3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (g5/14.1) deficiency, Hyper-IgM syndrome: X-linked (CD40 ligand deficiency) or non-X-linked, Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon g receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1 A/SAP deficiency), or hyper IgE syndrome.

16. The method of any one of claims 2 to 15, wherein the one or more DNMT is selected from DNMT1, DNMT3a, or DNMT3b, or an ortholog, homologue, variant, fragment, subsequence or derivative thereof.

17. The method of any one of claims 4 to 16, wherein the agent is selected from the group of: an agent that promotes the activity of the one or more DNMTs or TETs at the heterochromatin in the cell; an agent that transports the one or more DNMT or TETs to the heterochromatin in the cell; an agent that increases the binding of the one or more DNMT or TETs to the heterochromatin in the cell; an agent that activates the expression of the one or more DNMT or TETs by the cell; or an agent comprising or consisting of DNMT1, DNMT3a, or DNMT3b, or an ortholog, homologue, variant, fragment, subsequence or derivative thereof; or an agent comprising or consisting of TET1, TET2, or TET3, or an ortholog, homologue, variant, fragment, subsequence or derivative thereof.

18. The method of any one of claims 4 to 17, wherein the agent is a small molecule, a ligand, an antibody, antibody fragment or mimetic, a protein, a fusion protein, a peptide, a nucleotide or a small interfering RNA.

19. The method of claim 18, wherein the agent is an antibody that binds to DNMT or a DNMT ligand, an agent that activates a DNMT gene, or a prodrug or solvate thereof.

20. The method of any one of claims 4 to 19, wherein the agent modulates the one or more DNMT by promoting the trafficking of the one or more DNMTs or one or more TETs to the heterochromatin of the cell.

21. The method of claim 19 or claim 20, wherein the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof.

22. The method of claim 21, wherein the agent comprises heterochromatin protein 1 (hp1b), or an ortholog, homologue, variant, fragment, subsequence or derivative thereof.

23. The method of any one of claims 17 to 22, wherein the agent is administered prior to, contemporaneous with, or after diagnosis or treatment of the neoplasia, neoplastic disorder, tumor, cancer or malignancy; metastasis of a neoplasia, tumor, cancer or malignancy to other sites; formation or establishment of a metastatic neoplasia, neoplastic disorder, tumor, cancer or malignancy to other sites distal from a primary neoplasia, neoplastic disorder, tumor, cancer or malignancy; or undesirable or aberrant age-associated genome dysfunction, immune disorder, or autoimmune response, disorder or disease.

24. A method of treating, preventing, reducing, suppressing, alleviating, or ameliorating an age-associated genome dysfunction in a subject in need thereof, the method comprising administering to the subject an agent that increases the expression of or activity of one or more DNMTs or TET proteins, or both, in the subject.

25. The method of claim 24, wherein the one or more DNMT is selected from DNMT1, DNMT3a, or DNMT3b, or an ortholog, homologue, variant, fragment, subsequence or derivative thereof.

26. The method of any one of claims 24 and 25, wherein the agent increases the activity of the one or more DNMTs, USP7, or TET proteins by promoting the activity of the one or more DNMTs, USP7, or TETs at a heterochromatin in the subject.

27. The method of any one of claims 24 to 26, wherein the agent is a DNMT or TET agonist.

28. The method of any one of claims 24 to 27, wherein the agent increases the activity of the one or more DNMT or TETs by promoting the trafficking of the one or more DNMT or TETs to the heterochromatin of the subject.

29. The method of any one of claims 24 to 28, wherein the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof.

30. The method of any one of claims 24 to 29, wherein the agent is a small molecule, a ligand, an antibody, antibody fragment or mimetic, a protein, a fusion protein, a peptide, a nucleotide or a small interfering RNA.

31. The method of claim 30, wherein the agent is an antibody that binds to DNMT or a DNMT ligand, a DNMT gene activating agent, or a prodrug or solvate thereof.

32. The method of claim 30 or claim 31, wherein the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof.

33. The method of claim 32, wherein the agent comprises heterochromatin protein 1 (hp1b), or an ortholog, homologue, variant, fragment, subsequence or derivative thereof.

34. The method of any one of claims 24 to 33, wherein the agent is administered prior to, contemporaneous with, or after diagnosis or treatment of the undesirable or aberrant age-associated genome dysfunction.

35. A method of treating, preventing, reducing, suppressing, alleviating, or ameliorating an immune disorder, or autoimmune response, disorder or disease in a subject in need thereof, the method comprising administering to the subject an agent that increases the expression of or activity of one or more DNA methyltransferases (DNMTs) proteins, an agent that increases the expression of or activity of one or more TET methyl-cytosine dioxygenases (TET) proteins, or both.

36. The method of claim 35, wherein the one or more DNMT is selected from DNMT1, DNMT3a, or DNMT3b, or an or an ortholog, homologue, variant, fragment, subsequence or derivative thereof; or wherein the one or more TET is selected from TET1, TET2, or TET3, or an ortholog, homologue, variant, fragment, subsequence or derivative thereof.

37. The method of any one of claims 35 to 36, wherein the agent increases the activity of the one or more DNMTs or one or more TETs by promoting the activity of the one or more DNMT at heterochromatin in the subject.

38. The method of any one of claims 35 to 37, wherein the agent is a DNMT or TET agonist.

39. The method of any one of claims 35 to 38, wherein the agent increases the activity of the one or more DNMTs or TETs by promoting the trafficking of the one or more DNMTs or TETs to the heterochromatin of the subject.

40. The method of any one of claims 35 to 39, wherein the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof.

41. The method of any one of claims 35 to 40, wherein the agent is a small molecule, a ligand, an antibody, antibody fragment or mimetic, a protein, a fusion protein, a peptide, a nucleotide or a small interfering RNA.

42. The method of claim 41, wherein the agent is an antibody that binds to DNMT or a DNMT ligand, a DNMT gene activating agent, or a prodrug or solvate thereof.

43. The method of claim 41 or claim 42, wherein the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof.

44. The method of claim 43, wherein the agent comprises heterochromatin protein 1 (hp1b), or an ortholog, homologue, variant, fragment, subsequence or derivative thereof.

45. The method of any one of claims 35 to 44, wherein the agent is administered prior to, contemporaneous with, or after diagnosis or treatment of the immune disorder, or autoimmune response, disorder or disease.

46. A method of treating cancer in a subject in need thereof, the method comprising administering to the subject an agent that increases the expression of or activates one or more DNMT in the subject.

47. A method of treating cancer in a subject in need thereof, the method comprising administering to the subject an agent that increases or promotes the activity one or more DNMT by promoting the trafficking of the one or more one or more DNA methyltransferase (DNMT) or one or more TET methyl-cytosine dioxygenases (TET) proteins, or both, to the heterochromatin in the cancer of the subject.

48. The method of claim 46 or claim 47, wherein the one or more DNMTs comprises or consists of DNMT1, DNMT3a, or DNMT3b, or an ortholog, homologue, variant, fragment, subsequence or derivative thereof; or one or more TETs comprises or consists of TET1, TET2, or TET3, or an ortholog, homologue, variant, fragment, subsequence or derivative thereof.

49. The method of any of claims 46 to 48, wherein the agent increases the activity of the one or more DNMT at the heterochromatin in the subject.

50. The method of any of claims 46 to 49, wherein the agent is an DNMT or TET agonist.

51. The method of any one of claims 46 to 50, wherein the agent is selected from the group of: an agent that promotes the activity of the one or more DNMT at the heterochromatin in the subject; an agent that transports the one or more DNMT to the heterochromatin in the subject; an agent that increases the binding of the one or more DNMT to the heterochromatin in the subject; an agent that activates the expression of the one or more DNMT in the subject; or an agent comprising or consisting of DNMT1, DNMT3a, or DNMT3b, or an ortholog, homologue, variant, fragment, subsequence or derivative thereof; or an agent comprising or consisting of TET1, TET2, or TET3, or an ortholog, homologue, variant, fragment, subsequence or derivative thereof.

52. The method of any of claims 46 to 51, wherein the agent is a small molecule, a ligand, an antibody, antibody fragment or mimetic, a protein, a fusion protein, a peptide, a nucleotide or a small interfering RNA.

53. The method of claim 52, wherein the agent is an antibody that binds to DNMT or a DNMT ligand, a DNMT gene activating agent, or a prodrug or solvate thereof.

54. The method of claim 52 or claim 53, wherein the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof.

55. The method of claim 54, wherein the agent comprises heterochromatin protein 1 (hp1b), or an ortholog, homologue, variant, fragment, subsequence or derivative thereof.

56. The method of any one of claims 46 to 55, wherein the agent is administered prior to, contemporaneous with, or after treatment or diagnosis of the cancer.

57. The method of any of claims 4 to 56, wherein the administration is local or systemic.

58. The method of claim 57, wherein the administration comprises intravenous administration.

59. The method of any one of claims 1 to 58, wherein the subject is a mammal.

60. The method of claim 59, wherein the subject is a human patient.

61. The method of any one of claims 4 to 60, wherein the DNMT expression or activity, the TET expression or activity, or both, is prophylactically activated, elicited, stimulated, induced, promoted, increased or enhanced to increase, stimulate, induce, promote, enhance or maintain the genomic stability of the cell of the subject, wherein the cell is at least one of: a cancer cell, a cell with one or more unstable chromosomes, an aged cell, or a senescent cell.

62. A kit comprising an agent that modulates the activity of one or more DNMTs, one or more TETs, or both and instructions for use.

63. The kit of claim 62, wherein the one or more DNMT is selected from DNMT1, DNMT3a, or DNMT3b, or an ortholog, homologue, variant, fragment, subsequence or derivative thereof; and the one or more TETs comprising or consisting of TET1, TET2, or TET3, or an ortholog, homologue, variant, fragment, subsequence or derivative thereof, or combinations thereof.

64. The kit of claim 62 or 63, wherein the agent increases the activity of the one or more DNMTs or one or more TETs, or both, by promoting the trafficking of the one or more DNMTs, TETs, or both, to heterochromatin.

65. The kit of any one of claims 62-64, wherein the agent is an DNMT or TET agonist.

66. The kit of any one of claims 62-65, wherein the agent is selected from the group of: an agent that promotes the activity of the one or more DNMT or TETs at the heterochromatin in the subject; an agent that transports the one or more DNMT or TETs to the heterochromatin in the subject; an agent that increases the binding of the one or more DNMT or TETs to the heterochromatin in the subject; an agent that activates the expression of the one or more DNMT or TETs in the subject; or an agent that activates the expression of the one or more DNMTs comprising or consisting of DNMT1, DNMT3a, or DNMT3b, or an ortholog, homologue, variant, fragment, subsequence or derivative thereof; or an agent that activates the expression of the one or more TETs comprising or consisting of TET1, TET2, or TET3, or an ortholog, homologue, variant, fragment, subsequence or derivative thereof.

67. The kit of any one of claims 62-66, wherein the agent is a small molecule, a ligand, an antibody, antibody fragment or mimetic, a protein, a fusion protein, a peptide, a nucleotide or a small interfering RNA.

68. The kit of claim 67, wherein the agent is an antibody that binds to DNMT or a DNMT ligand, a DNMT gene activating agent, or a prodrug or solvate thereof.

69. The kit of claim 67 or claim 68, wherein the agent comprises a fusion protein or a nucleic acid molecule encoding a fusion protein, wherein the fusion protein comprises a histone binding protein, or an ortholog, homologue, variant, fragment, subsequence or derivative thereof, and the one or more DNMT, or an ortholog, homologue, variant, fragment, subsequence or derivative thereof.

70. The kit of claim 69, wherein the agent comprises heterochromatin protein 1 (hp1b), or an ortholog, homologue, variant, fragment, subsequence or derivative thereof.

71. The kit of any one of claims 62-70, wherein the agent is administered prior to, contemporaneous with, or after diagnosis or treatment.
